(19) United States
(12) Patent Application Publication
(10) Pub. No.: US 2023/0340470 A1
(43) Pub. Date: Oct. 26, 2023

(54) METHODS FOR TREATING HUNTINGTON'S DISEASE

(71) Applicants: ASKLEPIOS BIOPHARMACEUTICAL, INC., Research Triangle Park, NC (US); THE UNIVERSITY OF NORTH CAROLINA AT CHAPEL HILL, Chapel Hill, NC (US)

(72) Inventors: SiewHui LOW, Research Triangle Park, NC (US); Xiao XIAO, Chapel Hill, NC (US); Philippe MOULLIER, Paris (FR); Bin XIAO, Chapel Hill, NC (US)

(73) Assignees: ASKLEPIOS BIOPHARMACEUTICAL, INC., Research Triangle Park, NC (US); THE UNIVERSITY OF NORTH CAROLINA AT CHAPEL HILL, Chapel Hill, NC (US)

(21) Appl. No.: 17/786,932

(22) PCT Filed: Dec. 18, 2020
(86) PCT No.: PCT/US2020/066039

§ 371 Date: Jun. 17, 2022

Related U.S. Application Data
(60) Provisional application No. 62/951,582, filed on Dec. 20, 2019.

(51) Int. Cl. C12N 15/113; C12N 15/86; C12N 9/02; A61P 25/28; A61K 48/00

(52) U.S. Cl. CPC C12N 15/113; C12N 15/86; C12N 9/0081; A61P 25/28; A61K 48/0066

(57) ABSTRACT

Aspects of the disclosure relate to compositions and methods useful for treating Huntington's disease. In some embodiments, the disclosure provides interfering nucleic acids (e.g., artificial miRNAs) targeting the huntingtin gene (HTT) and methods of treating Huntington's disease using the same. Accordingly, in some aspects, the disclosure provides an isolated nucleic acid comprising or encoding the sequence set forth in any one of SEQ ID NOs: 1-22.

### CROSS-REFERENCE TO RELATED APPLICATIONS

This application claims benefit under 35 U.S.C. § 119(e) of U.S. Provisional Application No. 62/951,582 filed Dec. 20, 2019, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Dec. 17, 2020, is named 046192-096790WOPT_SL.txt and is 40,419 bytes in size.

### TECHNICAL FIELD

The technology described herein relates to methods for treating Huntington's disease.

### BACKGROUND

Huntington's disease (HD) is a devastating inherited neurodegenerative disease caused by an expansion of the CAG repeat region in exon 1 of the huntingtin gene. While the Huntingtin protein (HTT) is expressed throughout the body, the polyglutamine expanded protein is especially toxic to medium spiny neurons in the striatum and their cortical connections. Patients struggle with emotional symptoms including depression and anxiety and with characteristic movement disturbances and chorea. There is currently no cure for Huntington's disease; therapeutic options are limited to ameliorating disease symptoms.

### SUMMARY

Aspects of the disclosure relate to compositions and methods useful for treating Huntington's disease (HD). In some embodiments, inhibitory nucleic acids (e.g., miRNAs, such as artificial miRNAs) are provided that hybridize specifically to and inhibit expression of human huntingtin (HTT).

Accordingly, in some aspects, the disclosure provides an isolated nucleic acid comprising or encoding the sequence set forth in any one of SEQ ID NOs: 1-22.

In one aspect described herein is an isolated nucleic acid comprising: (a) a first region comprising a first adeno-associated virus (AAV) inverted terminal repeat (ITR), or a variant thereof; and (b) a second region comprising a transgene encoding one or more miRNAs, wherein each miRNA comprises a seed sequence complementary to SEQ ID NO: 25.

In one aspect described herein is an isolated nucleic acid comprising: (a) a first region comprising a first adeno-associated virus (AAV) inverted terminal repeat (ITR), or a variant thereof; and (b) a second region comprising a transgene encoding one or more miRNAs, wherein each miRNA is encoded by a sequence comprising the sequence set forth in any one of SEQ ID NOs: 1-22 flanked by a miRNA backbone sequence.

In some aspects, the disclosure provides an isolated nucleic acid comprising: a first region comprising a first adeno-associated virus (AAV) inverted terminal repeat (ITR), or a variant thereof; and, a second region comprising a transgene encoding one or more miRNAs.

In some embodiments, the sequence encoding each miRNA comprises a sequence set forth in any one of SEQ ID NOs: 1-22. In some embodiments, the sequence encoding each miRNA comprises a sequence set forth in any one of SEQ ID NOs: 1-22 flanked by sequence from the pre-miR. In some embodiments, the isolated nucleic acid comprises pre-miR sequences corresponding to the mature miRNA sequence set forth in any one of SEQ ID NOs: 1-22. In some embodiments, the sequence encoding each miRNA comprises a sequence set forth in any one of SEQ ID NOs: 1-22 flanked by sequence encoding a miRNA backbone sequence. In some aspects, the disclosure provides an isolated nucleic acid comprising a transgene encoding one or more miRNAs, wherein the sequence of the transgene encoding each miRNA comprises a sequence set forth in SEQ ID NOs: 1-22 flanked by a miRNA backbone sequence.

In some embodiments, the transgene comprises two miRNA in tandem that are flanked by introns. In some embodiments, the transgene comprises two precursor miRNAs pre-miRNA in tandem (see e.g., SEQ ID NO: 35) that are flanked by introns.

In some embodiments, the transgene comprises two miRNA or two precursor miRNAs in tandem that are flanked by introns.

In some embodiments, the flanking introns are identical.

In some embodiments, the flanking introns are from the same species.

In some embodiments, the flanking introns are hCG introns.

In some embodiments, the transgene further comprises a nucleic acid sequence encoding a promoter.

In some embodiments, the promoter is a synapsin (Syn1) promoter.

In some embodiments, the transgene further comprises a nucleic acid sequence encoding a protein.

In some embodiments, the protein is CYP46A1.

In some embodiments, the protein is a therapeutic protein (e.g., non-mutant huntingtin) or a reporter protein (e.g., a fluorescent protein, such as GFP).

In some embodiments, human huntingtin comprises a sequence as set forth in SEQ ID NO: 25.

In some embodiments, the disclosure provides a nucleic acid (e.g., a miRNA) that is complementary to at least two (e.g., 2, 3, 4, 5, 6, 7, 8, 9, 10, 11, 12, 13, 14, 15, 16, 17, 18, 19, 20, 21, 22, 23, 24 or 25) continuous bases of SEQ ID NO: 25.

In some embodiments, the one or more miRNAs is located in an untranslated portion of the transgene.

In some embodiments, the untranslated portion is an intron.

In some embodiments, the untranslated portion is between the last codon of the nucleic acid sequence encoding a protein and a poly-A tail sequence.

In some embodiments, the untranslated portion is between the last nucleic acid base of a promoter sequence and the first base of a poly-A tail sequence.

In some embodiments, the polyA sequence is a small PolyA sequence.

In some embodiments, the transgene is flanked by adeno-associated virus (AAV) inverted terminal repeats (ITRs), or variants thereof.

In some embodiments, the isolated nucleic acid further comprises a third region that comprises a second adeno-associated virus (AAV) inverted terminal repeat (ITR), or a variant thereof.

In some embodiments, the first or second ITR variant lacks a functional terminal resolution site (TRS), optionally wherein the ITR variant is a ATRS ITR.

In some embodiments, at least one of the miRNAs hybridizes with and inhibits expression of human huntingtin (e.g., SEQ ID NO: 25).

In some aspects, the disclosure provides a vector comprising an isolated nucleic acid as described by the disclosure.

In some aspects, the disclosure provides a vector comprising an isolated nucleic acid comprising a transgene encoding one or more miRNAs, wherein the sequence of the transgene encoding each miRNA comprises a sequence set forth in SEQ ID NOs: 1-22 flanked by a miRNA backbone sequence.

In some embodiments, the vector is a plasmid.

In some embodiments, each miRNA backbone sequence of the transgene is a mir-155 backbone sequence, a mir-30 backbone sequence, or a mir-64 backbone sequence.

In some aspects, the disclosure provides a host cell comprising an isolated nucleic acid or a vector as described by the disclosure.

In some aspects, the disclosure provides a recombinant AAV (rAAV) comprising: (a) a capsid protein; and (b) an isolated nucleic acid as described by the disclosure.

In some aspects, the disclosure provides a recombinant AAV (rAAV) comprising a capsid protein; and, an isolated nucleic acid comprising a transgene encoding one or more miRNAs, wherein the sequence of the transgene encoding each miRNA comprises a sequence set forth in SEQ ID NOs: 1-22 flanked by a miRNA backbone sequence.

In some embodiments, the capsid protein is an AAV9 capsid protein.

In some embodiments, the capsid protein is an AAVrh10 capsid protein.

In some embodiments, the capsid protein is an AAV1, AAV2, AAV3b, AAV4, AAV5, AAV6, AAV7, AAV8, AAV9, AAV10, AAV11, AAV12, or AAV13, or AAVrh10 capsid protein, or any chimera thereof.

In some embodiments, recombinant AAV (rAAV) is a haploid rAAV.

In some embodiments, the haploid rAAV comprises chimeric capsid proteins.

In some embodiments, the rAAV is a self-complementary AAV (scAAV).

In some embodiments, the rAAV is formulated for delivery to the central nervous system (CNS).

Aspects of the disclosure relate to a composition comprising any of the isolated nucleic acids described herein.

Aspects of the disclosure relate to a composition comprising any of the vectors described herein.

Aspects of the disclosure relate to a composition comprising any of the rAAV described herein.

Aspects of the disclosure relate to isolated nucleic acids capable of reducing (e.g., inhibiting) expression of pathogenic huntingtin and thus may be useful for the treatment of Huntington's disease.

Accordingly, in some aspects, the disclosure provides a method for treating Huntington's disease in a subject in need thereof, the method comprising administering to a subject having or at risk of developing Huntington's disease a therapeutically effective amount of an isolated nucleic acid, an rAAV, or a composition as described by the disclosure.

In some aspects, the disclosure provides a method for treating Huntington's disease in a subject in need thereof, the method comprising administering to a subject having or at risk of developing Huntington's disease a therapeutically effective amount of an rAAV as described herein (e.g., an rAAV comprising a transgene encoding one or more miRNAs, wherein the sequence of the transgene encoding each miRNA comprises a sequence set forth in SEQ ID NO: 1-22 flanked by a miRNA backbone sequence).

In some embodiments, the subject comprises a huntingtin gene having more than 36 CAG repeats, more than 40 repeats, or more than 100 repeats.

In some embodiments, the subject is less than 20 years of age, or is diagnosed as having juvenile HD.

In some embodiments, the administration results in delivery of the isolated nucleic acid or rAAV to the central nervous system (CNS) of the subject.

In some embodiments, the administration is via injection, optionally intravenous injection or intrastriatal injection.

In some embodiments, the administration is via catheter or related device.

In some embodiments of any aspect, the method further comprising the step, prior to administering, of diagnosing a subject as having or at risk of developing Huntington's disease.

In some embodiments of any aspect, further comprising the step, prior to administering, of receiving the results of an assay that diagnoses a subject as having or at risk of developing Huntington's disease. Exemplary assays for diagnosing a subject as having or at risk of developing Huntington's disease are described herein, e.g., genetic screening for at least 36 CAG repeats, at least 40 CAG repeats, or at least 100 CAG repeats, or more.

### BRIEF DESCRIPTION OF THE DRAWINGS

FIG. 1 is a schematic showing the construct of artificial miRNAs. pEMBL-D(+)-Syn1-hCG intron is a control vector, which is inserted with empty human chorionic gonadotropin (hCG) intron and driven with synapsin promoter. Two copies of control miRNA precursor (random sequences or non-functional mutation) are inserted into hCGin in the vector pEMBL-D(+)-Syn1-hCGin-2×control pre-miR. Two copies of artificial pre-miR (perfect match with 3′-UTR targeting sequences, including about 100-150 bp flanked upstream and downstream sequences) are cloned into between the hCG introns. The vector pEMBL-D(+)-Syn1-CYP46A1-hCGin-2×artificial pre-miR is a combo construct, which could produce both CYP46A1 and artificial miRNA at the same time. In order to identify whether the pre-miRNA could be processed into mature miRNA and combined with HTT targeting sequences including CAG expansions, which are perfectly complementary with mature miRNA, are inserted behind luciferase gene. For the limit of package size, small poly A is used in the constructs.

FIG. 2 is a schematic showing mechanisms of Huntington's Disease (HD).

FIG. 3 is a schematic showing exemplary methods of HD treatment.

FIG. 4 is a schematic showing process for screening artificial miRNAs for HD.

FIG. 5 is a schematic showing the artificial miRNAs' locations in the HTT gene (or mRNA). miHTT-H2 is located at region I; miHTT-H4 and miHTT-H5 are located at the 5′ and 3′ jumpers of the CAG repeats; miHTT-H14 is located at region IV; miHTT-H15, H17, H19 and H21 are located at region V.

FIG. 6 is a schematic showing the regions of the HTT gene. CAG Repeats are located in region I.

FIG. 7 is a schematic showing the first screening of artificial miRNAs via plasmid transfection in vitro (e.g., in 293 cell line; Phase I).

FIG. 8A-8B is a series of schematics and graphs showing the first screening of artificial miRNAs via plasmid transfection in vitro in 293 cells (e.g., Phase I). FIG. 8A is a schematic showing selected artificial miRNAs and their target regions on the HTT gene. FIG. 8B is a bar graph showing that the artificial miRNAs inhibited luciferase gene expression with targeting sequences by co-transfection. After 48-hour co-transfection, pEMBL-CMV-hCGin-miHTT-H2 and miHTT-H5 could efficiently inhibit luciferase activity about 46.4% and 54.8%, individually, compared with pEMBL-CMV-hCGin (as a controls). **p<0.01 vs Pembl-CMV-hCGin.

FIG. 9 is a schematic showing a process for screening artificial miRNAs for HD (e.g., Phase I).

FIG. 10 is a schematic showing the second screening of artificial miRNAs via AAV infection in vitro.

FIG. 11A-11B is a series of bar graphs showing the testing of AAVRH10 mediated-artificial miRNAs in the human neural cell line U87 (a human primary glioblastoma cell line). FIG. 11A shows the luciferase activity, and FIG. 11B shows the percentage of luciferase activity as compared to a control. FIG. 11A-11B show that AAVRH10 mediated-artificial miRNAs inhibited luciferase gene expression with targeting sequences in vitro. AAVRH10-CMV-hCGin-miHTT-H2 and H5 combined with their respective targeting sequences inserted into luciferase gene and greatly inhibited luciferase activity about 84.9% and 76.9%, compared with AAVRH10-CMV-hCGin (as a control). *p<0.05; ***p<0.001 vs. AAVRH10-CMV-hCGin.

FIG. 12 is a schematic showing the testing of artificial miRNAs' inhibition on HTT protein in the human neural cell U87 (e.g., Phase I).

FIG. 13 is presents western blots showing HTT protein levels in human neural cell line U87. After AAVRH10-CMV-hCGin-miHTT-H1-H5 treatment in U87 cells, HTT protein expression was reduced by AAVRH10-CMV-hCGin-miHTT-H2, -miHTT-H4 and -miHTT-H5. I3-actin is used as a loading control.

FIG. 14 is a bar graph showing the quantitative data of the HTT western blot in human neural cell line U87 (see e.g., FIG. 12). HTT protein expression was inhibited up to 73.2% by AAVRH10-CMV-hCGin-miHTT-H2, 58.5% by miHTT4 and 41.5% by miHTT-H5. (*p<0.05, **p<0.01, n=4).

FIG. 15 is a schematic showing a process for screening artificial miRNAs for HD (e.g., Phase II).

FIG. 16 is a schematic showing the first screening of artificial miRNAs via plasmid transfection in vitro (e.g., in 293 cell line; Phase II).

FIG. 17 is a bar graph showing the second round screening with sequences from 3′-UTR. Artificial miRNAs inhibited luciferase gene expression with targeting sequences by co-transfection. In the second round screening, after 48-hour co-transfection, pEMBL-CMV-hCGin-miHTT-H14, H15, H17, H19 (miR-137) and miHTT-H21 (miR-216) efficiently inhibited luciferase activity compared with pEMBL-CMV-hCGin (as a control). MiHTT-H2, H4 and H5 were used as positive controls. MiDMPK-M5, M7 and M9 for targeting the dystrophia myotonica protein kinase (DMPK) gene were also used as negative controls. ***p<0.001 vs. pEMBL-CMV-hCGin.

FIG. 18 is a bar graph showing the second round screening with sequences from 3′-UTR. Artificial miRNAs inhibited luciferase gene expression with targeting sequences by co-transfection. After 48-hour co-transfection, pEMBL-CMV-hCGin-miHTT-H14, H15, H17, H19 (miR-137) and miHTT-H21 (miR-216) efficiently inhibited luciferase activity compared with pEMBL-CMV-hCGin (as a control). Related to the control (100%), the activity of luciferase was 2.45% (H14), 8.75% (H15), 9.2% (H17), 12.89% (miR-137), and 4.17% (miR-216), individually. MiHTT-H2, H4 and H5 were used as positive controls. MiDMPK-M5, M7 and M9 for targeting the DMPK gene were also used as negative controls. ***p<0.001 vs. pEMBL-CMV-hCGin.

FIG. 19 is a schematic showing the second round screening with sequences from 3′-UTR. Specifically, the schematic shows the artificial miRNAs' locations in the HTT gene. miHTT-H2 is located at region I; miHTT-H4 and miHTT-H5 are located at 5′ and 3′ jumper of CAG repeats, respectively; miHTT-H14 is located at region IV; and miHTT-H15, H17, H19 and H21 are located at region V.

FIG. 20 is a schematic showing the testing of artificial miRNAs' inhibition on HTT protein in the human neural cell U87 (e.g., Phase II).

FIG. 21 is a schematic showing the testing of artificial miRNAs in human fibroblast cell from HD patients. As a non-limiting example, the transfected/infected samples from the 2-3 highest performing miHTTs can be sent to perform off-target analysis.

FIG. 22 is a schematic showing artificial miRNA constructs. EMBL-D(+)-Syn1-hCGintron is a double-stranded vector that is used as a control and includes an empty human chorionic gonadotropin (hCG) intron (i.e., no miRNAs) driven with synapsin promoter. Two copies of artificial miHTT (perfect match with targeting sequences, including about 100-150 bp flanked upstream and downstream sequences) are cloned into between the hCG introns. In order to identify whether the miHTT could be processed into mature miRNA, HTT targeting sequences including CAG expansions, which are perfectly complementary with mature miRNA, are inserted behind luciferase gene. Due to the limit of package size, small poly A is used in the constructs. Note that Syn1 stands for synapsin 1.

FIG. 23 is a schematic showing a map of pEMBL-D(+)-Syn1-hCGin-2×miHTT.

FIG. 24 is a schematic showing the expression vectors of optimized CYP46A1. pAAV2.1-Syn1-GFP-sPA is a single-stranded vector used as a control. pAAV2.1-Syn1-CYP46A1-sPA is used for overexpressing CYP46A1, which is driven with muscle specific promoter Syn1. The vector pAAV2.1-Syn1-CYP46A1-hCGin-2×miHTT is a combo construct, which can produce both CYP46A1 and 2 copies of artificial miHTT at the same time.

FIG. 25 is a schematic showing a map of pAAV2.1-Syn1-CYP46A1-hCGin-2×miHTT.

FIG. 26 is a schematic showing the process of screening artificial miRNA and the identification of its targeting sequences in vitro. Two copies of artificial miRNA precursor are cut and processed into mature miRNA. The miRNA further exactly matches with HTT targeting sequences including CAG expansions and inhibits luciferase expression. At the same time, control miRNA can also be processed but could not combine with HTT targeting sequences, so it has no effect on the expression of luciferase. The method is usually utilized to identify the targeting sequence of miRNA in vitro.

FIG. 27 is a series of schematics and images showing the construct of artificial miRNAs based on the backbone of miR-30 precursor and associated blots.

### DETAILED DESCRIPTION

Aspects of the invention relate to certain interfering RNAs (e.g., miRNAs, such as artificial miRNAs) that when delivered to a subject are effective for reducing the expression of pathogenic huntingtin protein (HTT) in the subject. Accordingly, methods and compositions described by the disclosure are useful, in some embodiments, for the treatment of Huntington's disease.

### Inhibitory RNAs

In one aspect described herein are inhibitory RNAs that can be used for the treatment of Huntington's disease. In some embodiments of any of the aspects, the nucleic acid sequence of the inhibitory RNA comprises one of SEQ ID NOs: 1-24 or a sequence that is at least 95% (e.g., at least 95%, at least 96%, at least 97%, at least 98%, or at least 99%) identical to the sequence of at least one of SEQ ID NOs: 1-24 that maintains the same functions as at least one of SEQ ID NOs: 1-24 (e.g., HTT inhibition).

TABLE 1
Exemplary artificial miRNAs' location and sequences; with reference to
HTT mRNA (see e.g., SEQ ID NO: 25; e.g., Hu 128 TG mice)

| | Location of | | | | | Species of |
|---|---|---|---|---|---|---|
| | synthesized | | | | | sequences |
| | artificial | Number of | | Binding | Constructs of | targeted by |
| Regions | miRNAs | constructs | Name | sites | miRNAs | artificial miRNAs |
| Region I | CAG repeats | 5 | miHTT-H1 | >16 | GCTGCTGCTGC | Human/Non- |
| | | | | | TGCTGCTGC | human primate |
| | | | | | (SEQ ID NO: 1) | |
| | CAG repeats | | miHTT-H2 | >16 | TGCTGCTGCTG | Human/Non- |
| | | | | | CTGCTGCTG | human primate |
| | | | | | (SEQ ID NO: 2) | |
| | CAG repeats | | miHTT-H3 | >16 | GGCGGCGGCGG | Human/Non- |
| | | | | | CGGCGGCGG | human primate |
| | | | | | (SEQ ID NO: 3) | |
| | CAG 5′- | | miHTT-H4 | 1 | TGCTGGAAGGA | Human/Non- |
| | jumper | | | | CTTGAGGGA | human primate |
| | | | | | (SEQ ID NO: 4) | |
| | CAG 3′- | | miHTT-H5 | 1 | TGTTGCTGCTG | Human |
| | jumper | | | | CTGCTGCTG | |
| | | | | | (SEQ ID NO: 5) | |
| Region II | 5′-UTR | 2 | miHTT-H6 | 12 | CGAGGCCGGGG | Human/Non- |
| | | | | | CGGGGCACA | human primate |
| | | | | | (SEQ ID NO: 6) | |
| | | | miHTT-H7 | 3 | CGGGGCGGGGC | Human |
| | | | | | CGTGGAGGG | |
| | | | | | (SEQ ID NO: 7) | |
| Region III | Exon 1 | 3 | miHTT-H8 | 1 | ACTGTGCCACT | Human/Non- |
| | | | | | ATGTTTTCA | human primate |
| | | | | | (SEQ ID NO: 8) | |
| | | | miHTT-H9 | 1 | GCCTTCATCAG | Human/mouse/Non- |
| | | | | | CTTTTCCAG | human primate |
| | | | | | (SEQ ID NO: 9) | |
| | | | miHTT-H10 | 2 | GAGGGGTGGGG | Human/mouse/Non- |
| | | | | | AGGCTGGGG | human primate |
| | | | | | (SEQ ID NO: 10) | |
| Region IV | Exon 2-67 | 4 | miHTT-H11 | 1 | TCCTTGACCTG | Human/mouse/Non- |
| | | | | | CTGCTGCAG | human primate |
| | | | | | (SEQ ID NO: 11) | |
| | | | miHTT-H12 | 3 | CCTTCCACTGG | Human/mouse/Non- |
| | | | | | CCATGATGC | human primate |
| | | | | | (SEQ ID NO: 12) | |
| | | | miHTT-H13 | 2 | ACTGTGCCACT | Human/Non- |
| | | | | | ATGTTTTCA | human primate |
| | | | | | (SEQ ID NO: 13) | |
| | | | miHTT-H14 | 2 | TGAGGTATCAG | Human/Non- |
| | | | | | ATTGTCTAG | human primate |
| | | | | | (SEQ ID NO: 14) | |
| Region V | 3′-UTR | 4 | miHTT-H15 | 4 | AAAttAATCTCT | Human/mouse/Non- |
| | | | | | TACCTGAT | human primate |
| | | | | | (SEQ ID NO: 15) | |
| | | | miHTT-H16 | 1 | CCCAGGGCTAG | Human/Non- |
| | | | | | CAAGGAACA | human primate |
| | | | | | (SEQ ID NO: 16) | |
| | | | miHTT-H17 | 1 | AATTCAGTAGC | Human/mouse/Non- |
| | | | | | TTCCCTTAA | human primate |
| | | | | | (SEQ ID NO: 17) | |
| | | | miHTT-H18 | 1 | CTGGGCCCGCA | Human/Non- |
| | | | | | GCGGAAGGG | human primate |
| | | | | | (SEQ ID NO: 18) | |
| | | 4 | miHTT-H19 | 1 | TTATTGCTGTCT | Human/mouse/Non- |
| | | | (miR-137) | | ACTATCCG | human primate |
| | | | | | (SEQ ID NO: 19) | |
| | | | miHTT-H20 | 3 | TCAGTCCTTCCC | Human/Non- |
| | | | (miR-455) | | AAAGCTCT | human primate |
| | | | | | (SEQ ID NO: 20) | |
| | | | miHTT-H21 | 3 | TAATCTCTTTAC | Human/mouse/Non- |
| | | | (miR-216) | | TGATATAA | human primate |
| | | | | | (SEQ ID NO: 21) | |
| | | | miHTT-H22 | 3 | TCAGCAGTGTT | Human/mouse/Non- |
| | | | (miR-27a) | | ATTTCTTAC | human primate |
| | | | | | (SEQ ID NO: 22) | |
| | | 2 | miR-451a | 3 | AAACCGttACCAt | Human |
| | | | | | tACtGAGtt | |
| | | | | | (SEQ ID NO: 23) | |
| | | | miR-155 | 3 | AAAtCGCtGAtttG | Mouse |
| | | | | | tGtAGtC | |
| | | | | | (SEQ ID NO: 24) | |

Any combination of inhibitory RNAs described (e.g., SEQ ID NOs: 1-24) can be used, e.g., in vectors, rAAV compositions, or treatment methods as described herein. By way of non-limiting examples, the following combinations are specifically contemplated: at least one of SEQ ID NOs: 1-22; at least one of SEQ ID NOs: 1-10; at least one of SEQ ID NOs: 1-5; at least one of SEQ ID NOs: 6-7; at least one of SEQ ID NOs: 8-10; at least one of SEQ ID NOs: 11-24; at least one of SEQ ID NOs: 11-14; at least one of SEQ ID NOs: 15-24; at least one of SEQ ID NOs: 15-22; at least one of SEQ ID NOs: 15-18; at least one of SEQ ID NOs: 19-22; at least one of SEQ ID NOs: 23-24; at least one of SEQ ID NOs: 1 or 4-9; at least one of SEQ ID NOs: 2, 4, 5, 14, 15, 17, 19, or 21; at least one of SEQ ID NOs: 2, 4, 5, 14, 15, or 17; at least one of SEQ ID NOs: 2, 4, or 5; at least one of SEQ ID NOs: 2 or 5; or at least one of SEQ ID NOs: 14, 15, 17, 19, or 21; at least one of SEQ ID NOs: 14, 15, or 17.

In some embodiments of any of the aspects, the nucleic acid sequence of the inhibitory RNA comprises one of SEQ ID NOs: 1-22 or a sequence that is at least 95% (e.g., at least 95%, at least 96%, at least 97%, at least 98%, or at least 99%) identical to the sequence of at least one of SEQ ID NOs: 1-10 that maintains the same functions as at least one of SEQ ID NOs: 1-22 (e.g., HTT inhibition).

In some embodiments of any of the aspects, the inhibitory RNA targets at least one of Regions I-III (e.g., CAG repeats, CAG 5′ jumper, CAG 3′ jumper; 5′-UTR; or Exon 1) of the HTT gene. Accordingly, in some embodiments of any of the aspects, the nucleic acid sequence of the inhibitory RNA comprises one of SEQ ID NOs: 1-10 or a sequence that is at least 95% (e.g., at least 95%, at least 96%, at least 97%, at least 98%, or at least 99%) identical to the sequence of at least one of SEQ ID NOs: 1-10 that maintains the same functions as at least one of SEQ ID NOs: 1-10 (e.g., HTT inhibition).

In some embodiments of any of the aspects, the inhibitory RNA targets Region I of the HTT gene (e.g., CAG repeats, CAG 5′ jumper, or CAG 3′ jumper). Accordingly, in some embodiments of any of the aspects, the nucleic acid sequence of the inhibitory RNA comprises one of SEQ ID NOs: 1-5 or a sequence that is at least 95% (e.g., at least 95%, at least 96%, at least 97%, at least 98%, or at least 99%) identical to the sequence of at least one of SEQ ID NOs: 1-5 that maintains the same functions as at least one of SEQ ID NOs: 1-5 (e.g., HTT inhibition).

In some embodiments of any of the aspects, the inhibitory RNA targets Region II (e.g., 5′-UTR) of the HTT gene. Accordingly, in some embodiments of any of the aspects, the nucleic acid sequence of the inhibitory RNA comprises one of SEQ ID NOs: 6-7 or a sequence that is at least 95% (e.g., at least 95%, at least 96%, at least 97%, at least 98%, or at least 99%) identical to the sequence of at least one of SEQ ID NOs: 6-7 that maintains the same functions as at least one of SEQ ID NOs: 6-7 (e.g., HTT inhibition).

In some embodiments of any of the aspects, the inhibitory RNA targets Region III (Exon 1) of the HTT gene. Accordingly, in some embodiments of any of the aspects, the nucleic acid sequence of the inhibitory RNA comprises one of SEQ ID NOs: 8-10 or a sequence that is at least 95% (e.g., at least 95%, at least 96%, at least 97%, at least 98%, or at least 99%) identical to the sequence of at least one of SEQ ID NOs: 8-10 that maintains the same functions as at least one of SEQ ID NOs: 8-10 (e.g., HTT inhibition).

In some embodiments of any of the aspects, the inhibitory RNA targets at least one of Regions VI-V (e.g., Exon 2-67 or 3′ UTR) of the HTT gene. Accordingly, in some embodiments of any of the aspects, the nucleic acid sequence of the inhibitory RNA comprises one of SEQ ID NOs: 11-24 or a sequence that is at least 95% (e.g., at least 95%, at least 96%, at least 97%, at least 98%, or at least 99%) identical to the sequence of at least one of SEQ ID NOs: 11-24 that maintains the same functions as at least one of SEQ ID NOs: 11-24 (e.g., HTT inhibition).

In some embodiments of any of the aspects, the inhibitory RNA targets Region VI (e.g., Exon 2-67) of the HTT gene. Accordingly, in some embodiments of any of the aspects, the nucleic acid sequence of the inhibitory RNA comprises one of SEQ ID NOs: 11-14 or a sequence that is at least 95% (e.g., at least 95%, at least 96%, at least 97%, at least 98%, or at least 99%) identical to the sequence of at least one of SEQ ID NOs: 11-14 that maintains the same functions as at least one of SEQ ID NOs: 11-14 (e.g., HTT inhibition).

In some embodiments of any of the aspects, the inhibitory RNA targets Regions III and VI (e.g., 5′-UTR and Exon 2-67) of the HTT gene. Accordingly, in some embodiments of any of the aspects, the nucleic acid sequence of the inhibitory RNA comprises one of SEQ ID NOs: 8 or 13 or a sequence that is at least 95% (e.g., at least 95%, at least 96%, at least 97%, at least 98%, or at least 99%) identical to the sequence of at least one of SEQ ID NOs: 8 or 13 that maintains the same functions as at least one of SEQ ID NOs: 8 or 13 (e.g., HTT inhibition).

In some embodiments of any of the aspects, the inhibitory RNA targets Region V (e.g., 3′ UTR) of the HTT gene. Accordingly, in some embodiments of any of the aspects, the nucleic acid sequence of the inhibitory RNA comprises one of SEQ ID NOs: 15-24 or a sequence that is at least 95% (e.g., at least 95%, at least 96%, at least 97%, at least 98%, or at least 99%) identical to the sequence of at least one of SEQ ID NOs: 15-24 that maintains the same functions as at least one of SEQ ID NOs: 15-24 (e.g., HTT inhibition).

In some embodiments of any of the aspects, the inhibitory RNA targets Region V (e.g., 3′ UTR) of the HTT gene. Accordingly, in some embodiments of any of the aspects, the nucleic acid sequence of the inhibitory RNA comprises one of SEQ ID NOs: 15-22 or a sequence that is at least 95% (e.g., at least 95%, at least 96%, at least 97%, at least 98%, or at least 99%) identical to the sequence of at least one of SEQ ID NOs: 15-22 that maintains the same functions as at least one of SEQ ID NOs: 15-22 (e.g., HTT inhibition).

In some embodiments of any of the aspects, the inhibitory RNA targets Region V (e.g., 3′ UTR) of the HTT gene and is an artificial miRNA. Accordingly, in some embodiments of any of the aspects, the nucleic acid sequence of the inhibitory RNA comprises one of SEQ ID NOs: 15-18 or a sequence that is at least 95% (e.g., at least 95%, at least 96%, at least 97%, at least 98%, or at least 99%) identical to the sequence of at least one of SEQ ID NOs: 15-18 that maintains the same functions as at least one of SEQ ID NOs: 15-18 (e.g., HTT inhibition).

In some embodiments of any of the aspects, the inhibitory RNA targets Region V (e.g., 3′ UTR) of the HTT gene and is a human-expressed miRNA. Accordingly, in some embodiments of any of the aspects, the nucleic acid sequence of the inhibitory RNA comprises one of SEQ ID NOs: 19-22 or a sequence that is at least 95% (e.g., at least 95%, at least 96%, at least 97%, at least 98%, or at least 99%) identical to the sequence of at least one of SEQ ID NOs: 19-22 that maintains the same functions as at least one of SEQ ID NOs: 19-22 (e.g., HTT inhibition).

In some embodiments of any of the aspects, the inhibitory RNA targets Region V (e.g., 3′ UTR) of the HTT gene. Accordingly, in some embodiments of any of the aspects, the nucleic acid sequence of the inhibitory RNA comprises one of SEQ ID NOs: 23-24 or a sequence that is at least 95% (e.g., at least 95%, at least 96%, at least 97%, at least 98%, or at least 99%) identical to the sequence of at least one of SEQ ID NOs: 23-24 that maintains the same functions as at least one of SEQ ID NOs: 23-24 (e.g., HTT inhibition). In some embodiments of any of the aspects, the nucleic acid sequence of the inhibitory RNA does not comprise one of SEQ ID NOs: 23-24 or a sequence that is at least 95% (e.g., at least 95%, at least 96%, at least 97%, at least 98%, or at least 99%) identical to the sequence of at least one of SEQ ID NOs: 23-24 that maintains the same functions as at least one of SEQ ID NOs: 23-24 (e.g., HTT inhibition).

In some embodiments of any of the aspects, the nucleic acid sequence of the inhibitory RNA comprises one of SEQ ID NOs: 1 or 4-9, or a sequence that is at least 95% (e.g., at least 95%, at least 96%, at least 97%, at least 98%, or at least 99%) identical to the sequence of at least one of SEQ ID NOs: 1 or 4-9 that maintains the same functions as at least one of SEQ ID NOs: 1 or 4-9 (e.g., HTT inhibition).

In some embodiments of any of the aspects, the nucleic acid sequence of the inhibitory RNA comprises one of SEQ ID NOs: 2, 4, 5, 14, 15, 17, 19, 21, or a sequence that is at least 95% (e.g., at least 95%, at least 96%, at least 97%, at least 98%, or at least 99%) identical to the sequence of at least one of SEQ ID NOs: 2, 4, 5, 14, 15, 17, 19, 21 that maintains the same functions as at least one of SEQ ID NOs: 2, 4, 5, 14, 15, 17, 19, 21 (e.g., HTT inhibition).

In some embodiments of any of the aspects, the nucleic acid sequence of the inhibitory RNA comprises one of SEQ ID NOs: 2, 4, 5, 14, 15, 17, or a sequence that is at least 95% (e.g., at least 95%, at least 96%, at least 97%, at least 98%, or at least 99%) identical to the sequence of at least one of SEQ ID NOs: 2, 4, 5, 14, 15, 17 that maintains the same functions as at least one of SEQ ID NOs: 2, 4, 5, 14, 15, 17, (e.g., HTT inhibition).

In some embodiments of any of the aspects, the nucleic acid sequence of the inhibitory RNA comprises one of SEQ ID NOs: 2, 4, 5, or a sequence that is at least 95% (e.g., at least 95%, at least 96%, at least 97%, at least 98%, or at least 99%) identical to the sequence of at least one of SEQ ID NOs: 2, 4, 5 that maintains the same functions as at least one of SEQ ID NOs: 2, 4, 5 (e.g., HTT inhibition).

In some embodiments of any of the aspects, the nucleic acid sequence of the inhibitory RNA comprises one of SEQ ID NOs: 2, 5, or a sequence that is at least 95% (e.g., at least 95%, at least 96%, at least 97%, at least 98%, or at least 99%) identical to the sequence of at least one of SEQ ID NOs: 2 or 5 that maintains the same functions as at least one of SEQ ID NOs: 2 or 5 (e.g., HTT inhibition).

In some embodiments of any of the aspects, the nucleic acid sequence of the inhibitory RNA comprises one of SEQ ID NOs: 14, 15, 17, 19, 21, or a sequence that is at least 95% (e.g., at least 95%, at least 96%, at least 97%, at least 98%, or at least 99%) identical to the sequence of at least one of SEQ ID NOs: 14, 15, 17, 19, 21 that maintains the same functions as at least one of SEQ ID NOs: 14, 15, 17, 19, 21 (e.g., HTT inhibition).

In some embodiments of any of the aspects, the nucleic acid sequence of the inhibitory RNA comprises one of SEQ ID NOs: 14, 15, 17, or a sequence that is at least 95% (e.g., at least 95%, at least 96%, at least 97%, at least 98%, or at least 99%) identical to the sequence of at least one of SEQ ID NOs: 14, 15, 17 that maintains the same functions as at least one of SEQ ID NOs: 14, 15, 17 (e.g., HTT inhibition).

In some embodiments of any of the aspects, the nucleic acid sequence of the inhibitory RNA comprises miHTT-H2 (SEQ ID NO: 2). In some embodiments of any of the aspects, the nucleic acid sequence of the inhibitory RNA comprises miHTT-H4 (SEQ ID NO: 4). In some embodiments of any of the aspects, the nucleic acid sequence of the inhibitory RNA comprises miHTT-H5 (SEQ ID NO: 5). In some embodiments of any of the aspects, the nucleic acid sequence of the inhibitory RNA comprises miHTT-H14 (SEQ ID NO: 14). In some embodiments of any of the aspects, the nucleic acid sequence of the inhibitory RNA comprises miHTT-H15 (SEQ ID NO: 15). In some embodiments of any of the aspects, the nucleic acid sequence of the inhibitory RNA comprises miHTT-H17 (SEQ ID NO: 17). In some embodiments of any of the aspects, the nucleic acid sequence of the inhibitory RNA comprises miHTT-H19 (SEQ ID NO: 19; miR-137). In some embodiments of any of the aspects, the nucleic acid sequence of the inhibitory RNA comprises miHTT-H21 (SEQ ID NO: 21; miR-216).

In some embodiments of any of the aspects, the inhibitory RNA (e.g., miRNA) binds and/or targets at least one portion of an HTT nucleic acid (see e.g., SEQ ID NO: 25). In some embodiments of any of the aspects, the inhibitory RNA (e.g., miRNA) binds and/or targets the 5′ untranslated region the HTT nucleic acid (e.g., mRNA).

In some embodiments of any of the aspects, the inhibitory RNA (e.g., miRNA) binds and/or targets exon 1 (i.e., the first nucleic acid segment coding for a polypeptide) of the target (e.g., HTT).

In some embodiments of any of the aspects, the inhibitory RNA (e.g., miRNA) binds and/or targets CAG repeats of the HTT nucleic acid (e.g., mRNA). The term “CAG repeats” refers to the region of exon 1 of the HTT gene comprising a CAG trinucleotide (i.e., cytosine, adenine, and guanine) repeat. Normally, the CAG trinucleotide can be repeated 10 to 35 times within the HTT gene. In individuals with Huntington disease, the CAG segment can be repeated 36 to more than 120 times.

In some embodiments of any of the aspects, the inhibitory RNA (e.g., miRNA) binds and/or targets the CAG 5′-jumper of the HTT nucleic acid (e.g., mRNA). The term “CAG 5′-jumper” refers to the region of the HTT gene comprising the 3′ end of Exon 1 and the 5′ end of the CAG repeats.

In some embodiments of any of the aspects, the inhibitory RNA (e.g., miRNA) binds and/or targets a CAG 3′jumper of the HTT nucleic acid (e.g., mRNA). The term “CAG 3′-jumper” refers to the region of the HTT gene comprising the 3′ end of the CAG repeats and the 5′ end of Exon 2-67.

In some embodiments of any of the aspects, the inhibitory RNA (e.g., miRNA) binds and/or targets Exon 2-67 of the HTT nucleic acid (e.g., mRNA). The term “Exon 2-67” of the HTT gene refers to the region consisting of exon 2 to exon 67 of the HTT gene. In some embodiments of any of the aspects the inhibitory RNA (e.g., miRNA) binds and/or targets at least one of: exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, or exon 67 of the HTT nucleic acid (e.g., mRNA).

In some embodiments of any of the aspects, the inhibitory RNA (e.g., miRNA) binds and/or targets the 3′ untranslated region (UTR) of the HTT nucleic acid (e.g., mRNA).

In some embodiments of any of the aspects, the inhibitory RNA (e.g., miRNA) binds and/or targets the 5′ UTR, exon 1, CAG repeats, the CAG 5′-jumper, or a CAG 3′jumper of the target (e.g., HTT). In some embodiments of any of the aspects, the inhibitory RNA (e.g., miRNA) binds and/or targets the Exon 2-67 or the 3′ UTR of the target (e.g., HTT). In some embodiments of any of the aspects, the inhibitory RNA (e.g., miRNA) binds and/or targets the 5′ UTR, exon 1, CAG repeats, the CAG 5′-jumper, a CAG 3′jumper, Exon 2-67, or the 3′ UTR of the target (e.g., HTT).

In some embodiments of any of the aspects, the inhibitory RNA (e.g., miRNA) binds at least one binding site in the HTT nucleic acid (e.g., mRNA). In some embodiments of any of the aspects, the inhibitory RNA (e.g., miRNA) binds at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 binding sites in the HTT nucleic acid (e.g., mRNA).

In some embodiments of any of the aspects, the inhibitory RNA (e.g., miRNA) binds and/or targets the HTT nucleic acid (e.g., mRNA) of a human, a non-human primate, or a mouse. In some embodiments of any of the aspects, the inhibitory RNA (e.g., miRNA) binds and/or targets the HTT nucleic acid (e.g., mRNA) of a human, a non-human primate, and a mouse. In some embodiments of any of the aspects, the inhibitory RNA (e.g., miRNA) binds and/or targets the HTT nucleic acid (e.g., mRNA) of a human. In some embodiments of any of the aspects, the inhibitory RNA (e.g., miRNA) binds and/or targets the HTT nucleic acid (e.g., mRNA) of a non-human primate. In some embodiments of any of the aspects, the inhibitory RNA (e.g., miRNA) binds and/or targets the HTT nucleic acid (e.g., mRNA) of a mouse. In some embodiments of any of the aspects, the inhibitory RNA (e.g., miRNA) binds and/or targets the HTT nucleic acid (e.g., mRNA) of a human and a non-human primate.

In some embodiments of any of the aspects, the agent that treats Huntington's disease is an inhibitory nucleic acid. In some embodiments of any of the aspects, inhibitors of the expression of a given gene can be an inhibitory nucleic acid. As used herein, “inhibitory nucleic acid” refers to a nucleic acid molecule which can inhibit the expression of a target, e.g., double-stranded RNAs (dsRNAs), inhibitory RNAs (iRNAs), and the like.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject results in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Exemplary embodiments of types of inhibitory nucleic acids can include, e.g., siRNA, shRNA, miRNA, and/or amiRNA, which are well known in the art.

In some embodiments of any of the aspects, the inhibitory RNA for treating Huntington's disease is a miRNA. MicroRNAs (miRNAs) are small RNAs of 17-25 nucleotides, which function as regulators of gene expression in eukaryotes. miRNAs are initially expressed in the nucleus as part of long primary transcripts called primary miRNAs (pri-miRNAs). Inside the nucleus, pri-miRNAs are partially digested by the enzyme Drosha, to form 65-120 nucleotide-long hairpin precursor miRNAs (pre-miRNAs) that are exported to the cytoplasm for further processing by Dicer into shorter, mature miRNAs, which are the active molecules. In animals, these short RNAs comprise a 5′ proximal “seed” region (nucleotides 2 to 8) which appears to be the primary determinant of the pairing specificity of the miRNA to the 3′ untranslated region (3′-UTR) of a target mRNA. A more detailed explanation is given in the part dedicated to general definitions.

In the context of the invention, a miRNA molecule or an equivalent or a mimic or an isomiR thereof may be a synthetic or natural or recombinant or mature or part of a mature miRNA or a human miRNA or derived from a human miRNA as further defined in the part dedicated to the general definitions. A human miRNA molecule is a miRNA molecule which is found in a human cell, tissue, organ or body fluids (i.e. endogenous human miRNA molecule). A human miRNA molecule may also be a human miRNA molecule derived from an endogenous human miRNA molecule by substitution, deletion and/or addition of a nucleotide. A miRNA molecule or an equivalent or a mimic thereof may be a single stranded or double stranded RNA molecule. Preferably a miRNA molecule or an equivalent, or a mimic thereof is from 6 to 30 nucleotides in length, preferably 12 to 30 nucleotides in length, preferably 15 to 28 nucleotides in length, more preferably said molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

In a preferred embodiment, a miRNA molecule or equivalent or mimic or isomiR thereof comprises at least 6 of the 7 nucleotides present in the seed sequence of said miRNA molecule or equivalent or mimic or isomiR thereof. Preferably in this embodiment, a miRNA molecule or an equivalent or a mimic or isomiR thereof is from 6 to 30 nucleotides in length and more preferably comprises at least 6 of the 7 nucleotides present in the seed sequence of said miRNA molecule or equivalent thereof. Even more preferably a miRNA molecule or an equivalent or a mimic or isomiR thereof is from 15 to 28 nucleotides in length and more preferably comprises at least 6 of the 7 nucleotides present in the seed sequence, even more preferably a miRNA molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Accordingly, a preferred miRNA molecule or equivalent or mimic or isomiR thereof comprises at least 6 of the 7 nucleotides present in the seed sequence identified as at least one of SEQ ID NOs: 1-24 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Delivery vehicles for miRNA include but are not limited to the following: liposomes, polymeric nanoparticles, viral systems, conjugation of lipids or receptor-binding molecules, exosomes, and bacteriophage; see e.g., Baumann and Winkler, miRNA-based therapies: Strategies and delivery platforms for oligonucleotide and non-oligonucleotide agents, Future Med Chem. 2014, 6(17): 1967-1984; U.S. Pat. Nos. 8,900,627; 9,421,173; 9,555,060; WO 2019/177550; the contents of each of which are incorporated herein by reference in their entireties.

### Nucleic Acids

In some aspects, the disclosure provides isolated nucleic acids that are useful for reducing (e.g., inhibiting) expression of human huntingtin (HTT). A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more inhibitory RNAs (e.g., miRNAs) comprising a nucleic acid that targets an endogenous mRNA of a subject. The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV5, AAV6, AAV6.2, AAV7, AAV8, AAV9, AAV10, AAV11, AAVrh10, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV5, AAV6, AAV6.2, AAV7, AAV8, AAV9, AAV10, AAV11, AAVrh10, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10): 1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency {i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., miRNA).

In some aspects, the disclosure provides an isolated nucleic acid comprising a transgene, wherein the transgene comprises a nucleic acid sequence encoding one or more microRNAs (e.g., miRNAs). A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8, Pasha, etc.) into a pre-miRNA. The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than 200 base pairs in length (e.g., 2500, 5000, 7000, 9000, or more base pairs in length.

Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or 30 base pairs in length. In some embodiments, an isolated nucleic acid of the disclosure comprises a sequence encoding a pri-miRNA, a pre-miRNA, or a mature miRNA comprising a sequence set forth in any one of SEQ ID NOs: 1-24.

It should be appreciated that an isolated nucleic acid or vector (e.g., rAAV vector), in some embodiments comprises a nucleic acid sequence encoding more than one (e.g., a plurality, such as 2, 3, 4, 5, 10, or more) miRNAs. In some embodiments, each of the more than one miRNAs targets (e.g., hybridizes or binds specifically to) the same target gene (e.g., an isolated nucleic acid encoding three unique miRNAs, where each miRNA targets the HTT gene). In some embodiments, each of the more than one miRNAs targets (e.g., hybridizes or binds specifically to) different regions of the same target gene (e.g., HTT). In some embodiments, each of the more than one miRNAs targets (e.g., hybridizes or binds specifically to) a different target gene.

In some aspects, the disclosure provides isolated nucleic acids and vectors (e.g., rAAV vectors) that encode one or more artificial miRNAs. As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding amiRNA/amiRNA* sequences that direct highly efficient RNA silencing of the targeted gene, for example as described by Eamens et al. (2014), Methods Mol. Biol. 1062:211-224. For example, in some embodiments an artificial miRNA comprises a miR-155 pri-miRNA backbone into which a sequence encoding a mature HTT-specific miRNA (e.g., any one of SEQ ID NOs: 1-24) has been inserted in place of the endogenous miR-155 mature miRNA-encoding sequence. In some embodiments, miRNA (e.g., an artificial miRNA; e.g., one of SEQ ID NOs: 1-24) as described by the disclosure comprises a miR-155 backbone sequence, a miR-30 backbone sequence, a mir-64 backbone sequence, or a miR-122 backbone sequence. In some embodiments, miRNA (e.g., an artificial miRNA; e.g., one of SEQ ID NOs: 1-24) as described by the disclosure comprises a backbone as disclosed by SEQ ID NO: 35.

A region comprising a transgene (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon. In some cases (e.g., when a transgene lacks a protein coding sequence), it may be desirable to position the region (e.g., the second region, third region, fourth region, etc.) upstream of the poly-A tail of a transgene. For example, the region may be positioned between the first base of the poly-A tail and 2000 nucleotides upstream of the first base. The region may be positioned between the first base of the poly-A tail and 1000 nucleotides upstream of the first base. The region may be positioned between the first base of the poly-A tail and 500 nucleotides upstream of the first base. The region may be positioned between the first base of the poly-A tail and 250 nucleotides upstream of the first base. The region may be positioned between the first base of the poly-A tail and 150 nucleotides upstream of the first base. The region may be positioned between the first base of the poly-A tail and 100 nucleotides upstream of the first base. The region may be positioned between the first base of the poly-A tail and 50 nucleotides upstream of the first base. The region may be positioned between the first base of the poly-A tail and 20 nucleotides upstream of the first base. In some embodiments, the region is positioned between the last nucleotide base of a promoter sequence and the first nucleotide base of a poly-A tail sequence.

In some cases, the region may be positioned downstream of the last base of the poly-A tail of a transgene. The region may be between the last base of the poly-A tail and a position 2000 nucleotides downstream of the last base. The region may be between the last base of the poly-A tail and a position 1000 nucleotides downstream of the last base. The region may be between the last base of the poly-A tail and a position 500 nucleotides downstream of the last base. The region may be between the last base of the poly-A tail and a position 250 nucleotides downstream of the last base. The region may be between the last base of the poly-A tail and a position 150 nucleotides downstream of the last base.

It should be appreciated that in cases where a transgene encodes more than one miRNA, each miRNA may be positioned in any suitable location within the transgene. For example, a nucleic acid encoding a first miRNA may be positioned in an intron of the transgene and a nucleic acid sequence encoding a second miRNA may be positioned in another untranslated region (e.g., between the last codon of a protein coding sequence and the first base of the poly-A tail of the transgene).

In some embodiments, the transgene further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available (see, e.g., Sambrook et al., and references cited therein at, for example, pages 3.18 3.26 and 16.17 16.27 and Ausubel et al., Current Protocols in Molecular Biology, John Wiley & Sons, New York, 1989). In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, Petal., Human Gene Therapy, 2000; 11: 1921-1931; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al., Cell, 41:521-530 (1985)), the SV40 promoter, the dihydrofolate reductase promoter, the β-actin promoter, the phosphoglycerol kinase (PGK) promoter, and the EF1 a promoter (Invitrogen). In some embodiments, a promoter is an enhanced chicken β-actin promoter. In some embodiments, a promoter is a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268: 1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3: 1002-9 (1996); alpha-fetoprotein (AFP) promoter, Arbuthnot et al., Hum. Gene Ther., 7: 1503-14 (1996)), bone osteocalcin promoter (Stein et al., Mol. Biol. Rep., 24: 185-96 (1997)); bone sialoprotein promoter (Chen et al., J. Bone Miner. Res., 11:654-64 (1996)), CD2 promoter (Hansal et al., J. Immunol., 161: 1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding an inhibitory RNA (e.g., miRNA), it may be desirable to drive expression of the protein coding region using a first promoter sequence (e.g., a first promoter sequence operably linked to the protein coding region), and to drive expression of the inhibitory RNA encoding region with a second promoter sequence (e.g., a second promoter sequence operably linked to the inhibitory RNA encoding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (polIII) promoter sequence. Non-limiting examples of polIII promoter sequences include U6 and HI promoter sequences. In some embodiments, the second promoter sequence (e.g., the promoter sequence driving expression of the inhibitory RNA) is a RNA polymerase II (polII) promoter sequence. Non-limiting examples of polII promoter sequences include T7, T3, SP6, RSV, and cytomegalovirus promoter sequences. In some embodiments, a polIII promoter sequence drives expression of an inhibitory RNA (e.g., miRNA) encoding region. In some embodiments, a polII promoter sequence drives expression of a protein coding region.

In some embodiments, the nucleic acid comprises a transgene that encodes a protein. The protein can be a therapeutic protein (e.g., a peptide, protein, or polypeptide useful for the treatment or prevention of disease states in a mammalian subject) or a reporter protein. In some embodiments, the therapeutic protein is useful for treatment or prevention of Huntington's disease, for example CYP46A1, Polyglutamine binding peptide 1 (QBP1), PTD-QBP1, ED11, C4 intrabody, VL12.3 intrabody, MW7 intrabody, Happ1 antibodies, Happ3 antibodies, mEM48 intrabody, certain monoclonal antibodies (e.g., 1C2), and peptide P42 and variants thereof, as described in Marelli et al. (2016) Orphanet Journal of Rare Disease 11:24. In some embodiments, the therapeutic protein is wild-type huntingtin protein (e.g., huntingtin protein having a PolyQ repeat region comprising less than 36 repeats).

Without wishing to be bound by any particular theory, allele-specific silencing of mutant huntingtin (HTT) may provide an improved safety profile in a subject compared to non-allele specific silencing (e.g., silencing of both wild-type and mutant HTT alleles) because wild-type HTT expression and function is preserved in the cells. Aspects of the invention relate to the inventors' recognition and appreciation that isolated nucleic acids and vectors that incorporate one or more inhibitory RNA (e.g., miRNA) sequences targeting the HTT gene in a non-allele-specific manner while driving the expression of hardened wild-type HTT gene (a wild-type HTT gene that is not targeted by the miRNA) are capable of achieving concomitant mutant HTT knockdown e.g., in the CNS tissue, with increased expression of wild type HTT. Generally, the sequence of the nucleic acid encoding endogenous wild-type and mutant HTT mRNAs, and the nucleic acid of the transgene encoding the “hardened” wild-type HTT mRNA are sufficiently different such that the “hardened” wild-type HTT transgene mRNA is not targeted by the one or more inhibitory RNAs (e.g., miRNAs). This may be accomplished, for example, by introducing one or more silent mutations into the HTT transgene sequence such that it encodes the same protein as the endogenous wild-type HTT gene but has a different nucleic acid sequence. In this case, the exogenous mRNA may be referred to as “hardened.” Alternatively, the inhibitory RNA (e.g., miRNA) can target the 5′ and/or 3′ untranslated regions of the endogenous wild-type HTT mRNA. These 5′ and/or 3′ regions can then be removed or replaced in the transgene mRNA such that the transgene mRNA is not targeted by the one or more inhibitory RNAs.

Reporter sequences (e.g., nucleic acid sequences encoding a reporter protein) that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of a nucleic acid. Recombinant adeno-associated viruses (rAAVs). In some aspects, the disclosure provides isolated AAVs. As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically, the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV2, AAV3, AAV4, AAV5, AAV6, AAV8, AAVrh8, AAV9, AAV10, and AAVrh10. In some embodiments, an AAV capsid protein is of a serotype derived from a non-human primate, for example AAVrh8 or AAVrh10 serotype. In some embodiments, an AAV capsid protein is of an AAV9 serotype. In some embodiments, an AAV capsid protein is of an AAVrh10 serotype. In some embodiments, the capsid protein is an AAV1, AAV2, AAV3b, AAV4, AAV5, AAV6, AAV7, AAV8, AAV9, AAV10, AAV11, AAV12, AAV13, or AAVrh10 capsid protein or any chimera thereof. In some embodiments, recombinant AAV (rAAV) is a haploid rAAV. In some embodiments, the haploid rAAV comprises chimeric capsid proteins.

In one embodiment, the viral capsid is modified. In one embodiment, the modified viral capsid is a chimeric capsid. A “chimeric’ capsid protein as used herein means an AAV capsid protein (e.g., any one or more of VP1, VP2 or VP3) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

In one embodiment, the modified viral capsid is a haploid capsid. As used herein, the term “haploid AAV” shall mean that AAV as described in International Application WO2018/170310, or US Application US2018/037149, which are incorporated herein in their entirety by reference. In some embodiments, a population of virions is a haploid AAV population where a virion particle can be constructed wherein at least one viral protein from the group consisting of AAV capsid proteins, VP1, VP2 and VP3, is different from at least one of the other viral proteins, required to form the virion particle capable of encapsulating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric is present. See e.g., US Patent Application 2019/0002841, or U.S. Pat. No. 8,906,675; the contents of each of which are incorporated herein by reference in their entireties.

The components to be cultured in the host cell to package a rAAV vector in an AAV capsid may be provided to the host cell in trans. Alternatively, any one or more of the required components {e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art. In some embodiments, the instant disclosure relates to a host cell containing a nucleic acid that comprises a coding sequence encoding a protein (e.g., wild-type huntingtin protein, optionally “hardened” wild-type huntingtin protein). In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13: 197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The foregoing methods for packaging recombinant vectors in desired AAV capsids to produce the rAAVs of the disclosure are not meant to be limiting and other suitable methods will be apparent to the skilled artisan.

In some embodiments, any one or more thymidine (T) nucleotides or uridine (U) nucleotides in a sequence provided herein, including a sequence provided in the sequence listing, may be replaced with any other nucleotide suitable for base pairing (e.g., via a Watson-Crick base pair) with an adenosine nucleotide. For example, in some embodiments, any one or more thymidine (T) nucleotides in a sequence provided herein, including a sequence provided in the sequence listing, may be suitably replaced with a uridine (U) nucleotide or vice versa.

In some embodiments of any of the aspects, a nucleic acid (e.g., miRNA) is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages. nucleic acids having modified backbones include, among others, those that do not have a phosphorus atom in the backbone. For the purposes of this specification, and as sometimes referenced in the art, modified nucleic acids that do not have a phosphorus atom in their internucleoside backbone can also be considered to be oligonucleosides. In some embodiments of any of the aspects, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

In other nucleic acid mimetics, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The nucleic acid can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol. Canc. Ther. 6(3):833-843; Grunweller, A. et al., (2003) Nucleic Acids Research 31(12):3185-3193).

Modified nucleic acids can also contain one or more substituted sugar moieties. The nucleic acids described herein can include one of the following at the 2′ position: OH; F; O—, S—, or N-alkyl; O—, S—, or N-alkenyl; O—, S- or N-alkynyl; or O-alkyl-Co-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2)nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, nucleic acids include one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid, or a group for improving the pharmacodynamic properties of a nucleic acid, and other substituents having similar properties. In some embodiments of any of the aspects, the modification includes a 2′ methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases can include other synthetic and natural nucleobases including but not limited to as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc. 2008, 130, 7, 2336-2343; Malyshev et. al. PNAS. 2012. 109 (30) 12005-12010). In some embodiments of any of the aspects, oligonucleotide tags (e.g., Oligopaint) comprise any modified nucleobases known in the art, i.e., any nucleobase that is modified from an unmodified and/or natural nucleobase.

The preparation of the modified nucleic acids, backbones, and nucleobases described above are well known in the art.

Another modification of a nucleic acid featured in the invention involves chemically linking to the nucleic acid to one or more ligands, moieties or conjugates that enhance the activity, cellular distribution, pharmacokinetic properties, or cellular uptake of the nucleic acid. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

### Treatment Methods

Methods for delivering a transgene (e.g., an inhibitory RNA, such as a miRNA) to a subject are provided by the disclosure. The methods typically involve administering to a subject an effective amount of an isolated nucleic acid encoding an interfering RNA capable of reducing expression of huntingtin (htt) protein, or a rAAV comprising a nucleic acid for expressing an inhibitory RNA capable of reducing expression of huntingtin protein.

In some aspects, the disclosure provides inhibitory miRNA that specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of human huntingtin (e.g., SEQ ID NO: 25). As used herein “continuous bases” refers to two or more nucleotide bases that are covalently bound (e.g., by one or more phosphodiester bond, etc.) to each other (e.g. as part of a nucleic acid molecule). In some embodiments, the at least one miRNA is about 50%, about 60% about 70% about 80% about 90%, about 95%, about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of SEQ ID NO: 25. In some embodiments, the inhibitory RNA is a miRNA which is comprises or is encoded by the sequence set forth in any one of SEQ ID NOs: 1-24.

As used herein, “Huntington's disease”, or “HD”, refers to a neurodegenerative disease characterized by progressively worsening movement, cognitive and behavioral changes caused by a tri-nucleotide repeat expansion (e.g., CAG, which is translated into a poly-Glutamine, or PolyQ, tract) in the HTT gene that results in production of pathogenic mutant huntingtin protein (HTT, or mHTT). In some embodiments, mutant huntingtin protein accelerates the rate of neuronal cell death in certain regions of the brain. Generally, the severity of HD is correlated to the size of the tri-nucleotide repeat expansion in a subject. For example, a subject having a CAG repeat region comprising between 36 and 39 repeats is characterized as having “reduced penetrance” HD, whereas a subject having greater than 40 repeats is characterized as having “full penetrance” HD. Thus, in some embodiments, a subject having or at risk of having HD has a HTT gene comprising between about 36 and about 39 CAG repeats (e.g., 36, 37, 38 or 39 repeats). In some embodiments, a subject having or at risk of having HD has a HTT gene comprising 40 or more (e.g., 40, 45, 50, 60, 70, 80, 90, 100, 200, or more) CAG repeats. In some embodiments, a subject having a HTT gene comprising more than 100 CAG repeats develops HD earlier than a subject having fewer than 100 CAG repeats. In some embodiments, a subject having a HTT gene comprising more than 100 CAG repeats may develop HD symptoms before the age of about 20 years, and is referred to as having juvenile HD (also referred to as akinetic-rigid HD, or Westphal variant HD). The number of CAG repeats in a HTT gene allele of a subject can be determined by any suitable modality known in the art. For example, nucleic acids (e.g., DNA) can be isolated from a biological sample (e.g., blood) of a subject and the number of CAG repeats of a HTT allele can be determined by a hybridization-based method, such as PCR or nucleic acid sequencing (e.g., Illumina sequencing, Sanger sequencing, SMRT sequencing, etc.).

In some embodiments of any aspect, the method further comprising the step, prior to administering, of diagnosing a subject as having or at risk of developing Huntington's disease.

In some embodiments of any aspect, further comprising the step, prior to administering, of receiving the results of an assay that diagnoses a subject as having or at risk of developing Huntington's disease. Exemplary assays for diagnosing a subject as having or at risk of developing Huntington's disease are described herein, e.g., genetic screening for at least 36 CAG repeats, at least 40 CAG repeats, or at least 100 CAG repeats, or more.

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is central nervous system (CNS) tissue (e.g., brain tissue, spinal cord tissue, cerebrospinal fluid (CSF), etc.). In some embodiments, an effective amount of an isolated nucleic acid (e.g., which may be delivered via an rAAV) may be an amount sufficient to have a therapeutic benefit in a subject, e.g., to reduce the expression of a pathogenic gene or protein (e.g., HTT), to extend the lifespan of a subject, to improve in the subject one or more symptoms of disease (e.g., a symptom of Huntington's disease), etc. The effective amount will depend on a variety of factors such as, for example, the species, age, weight, health of the subject, and the tissue to be targeted, and may thus vary among subject and tissue as described elsewhere in the disclosure.

### Administration

The rAAVs of the disclosure may be delivered to a subject in compositions according to any appropriate methods known in the art. For example, an rAAV, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, rAAV as described in the disclosure are administered by intravenous injection. In some embodiments, the rAAV are administered by intracerebral injection. In some embodiments, the rAAV are administered by intrathecal injection. In some embodiments, the rAAV are administered by intrastriatal injection. In some embodiments, the rAAV are delivered by intracranial injection. In some embodiments, the rAAV are delivered by cisterna magna injection. In some embodiments, the rAAV are delivered by cerebral lateral ventricle injection.

Aspects of the instant disclosure relate to compositions comprising a recombinant AAV comprising a capsid protein and a nucleic acid encoding a transgene, wherein the transgene comprises a nucleic acid sequence encoding one or more miRNAs. In some embodiments, each miRNA comprises a sequence set forth in any one of SEQ ID NOs: 1-24. In some embodiments, the nucleic acid further comprises AAV ITRs. In some embodiments, the ITR is an AAV1, AAV2, AAV3b, AAV4, AAV5, AAV6, AAV7, AAV8, AAV9, AAV10, AAV11, AAV12, AAV13, or AAVrh10 ITR. In some embodiments, a composition further comprises a pharmaceutically acceptable carrier. The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 109 to 1016 genome copies. In some cases, a dosage between about 1011 to 1013 rAAV genome copies is appropriate. In certain embodiments, 1012 or 1013 rAAV genome copies is effective to target CNS tissue. In some cases, stable transgenic animals are produced by multiple doses of an rAAV.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., −10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1% or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 A, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the methods described herein relate to treating a subject having or diagnosed as having Huntington's disease with a nucleic acid described herein. Subjects having Huntington's disease can be identified by a physician using current methods of diagnosing Huntington's disease. Symptoms and/or complications of Huntington's disease which characterize these conditions and aid in diagnosis are well known in the art and include but are not limited to, depression and anxiety and with characteristic movement disturbances and chorea. Tests that may aid in a diagnosis of Huntington's disease, e.g. include, but are not limited to, genetic tests. A family history of Huntington's disease can also aid in determining if a subject is likely to have Huntington's disease or in making a diagnosis of Huntington's disease.

The compositions and methods described herein can be administered to a subject having or diagnosed as having Huntington's disease. In some embodiments, the methods described herein comprise administering an effective amount of compositions described herein, e.g. a nucleic acid described herein to a subject in order to alleviate a symptom of Huntington's disease. As used herein, “alleviating a symptom of Huntington's disease” is ameliorating any condition or symptom associated with Huntington's disease. As compared with an equivalent untreated control, such reduction is by at least 5%, 10%, 20%, 40%, 50%, 60%, 80%, 90%, 95%, 99% or more as measured by any standard technique.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for tumor growth and/or size among others. The dosage can be determined by a physician and adjusted, as necessary, to suit observed effects of the treatment.

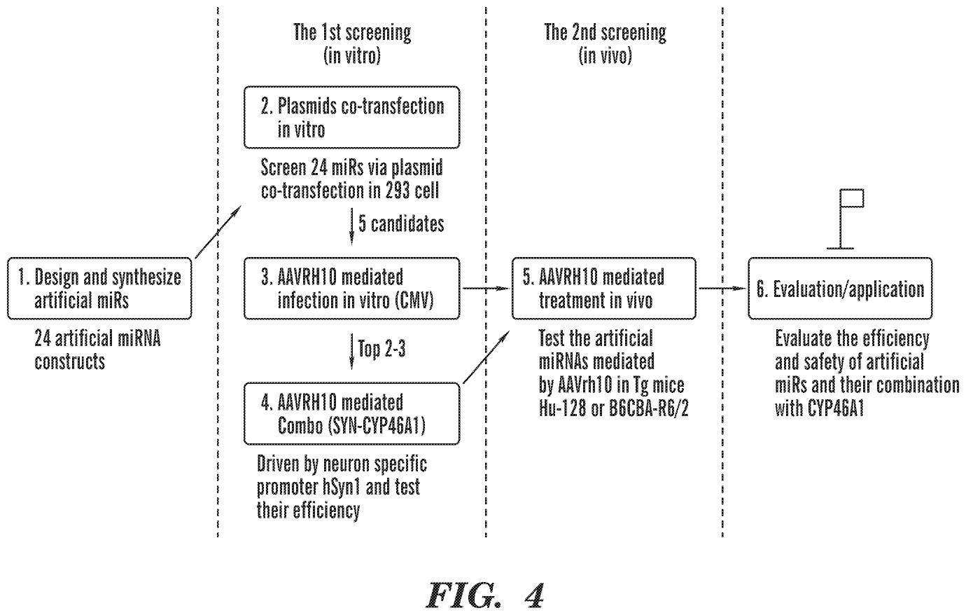

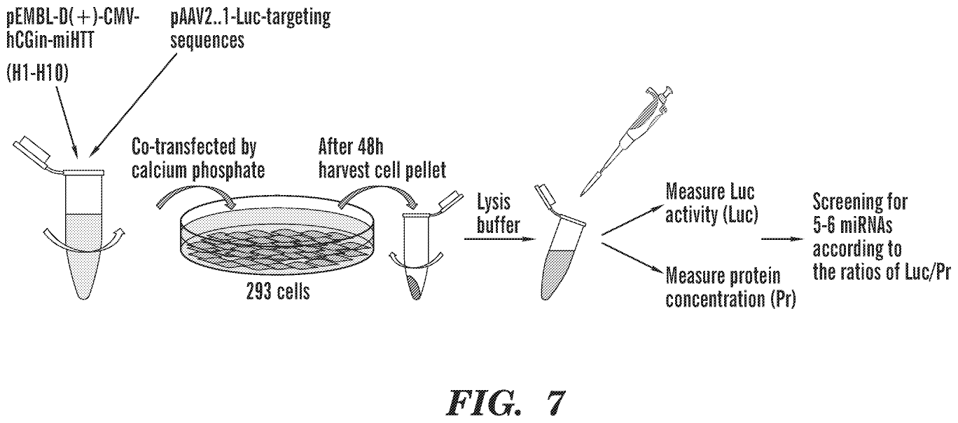

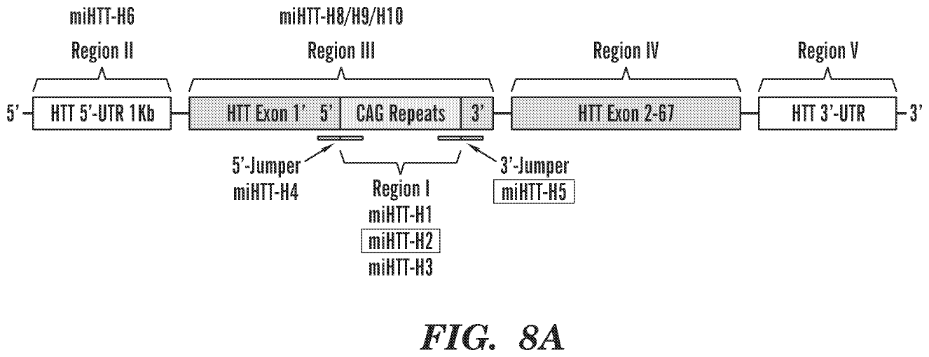

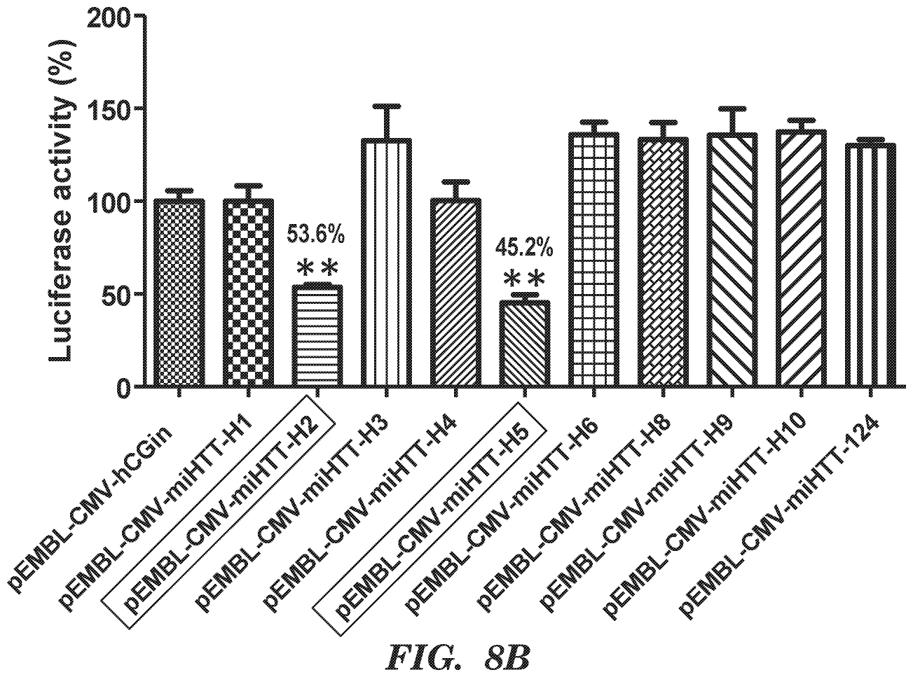

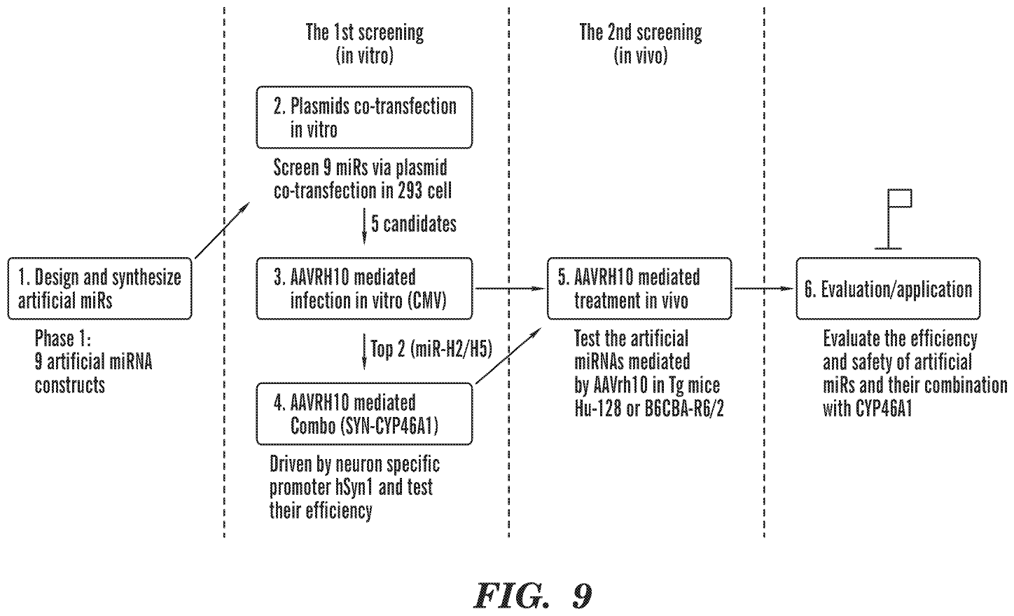

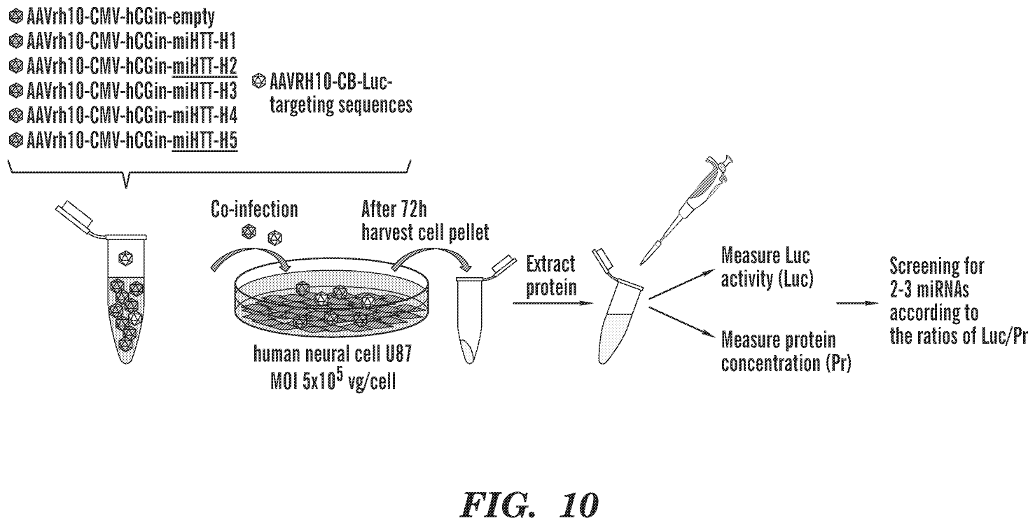

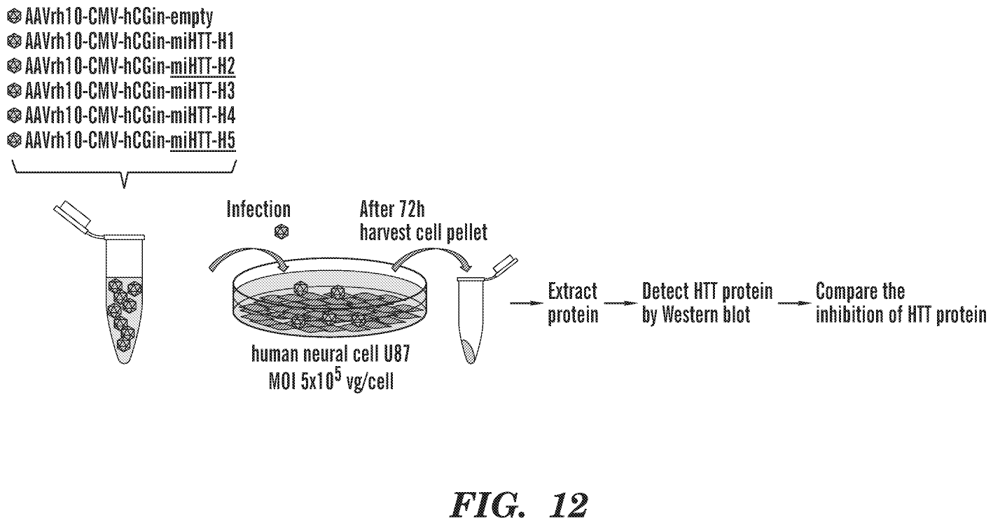

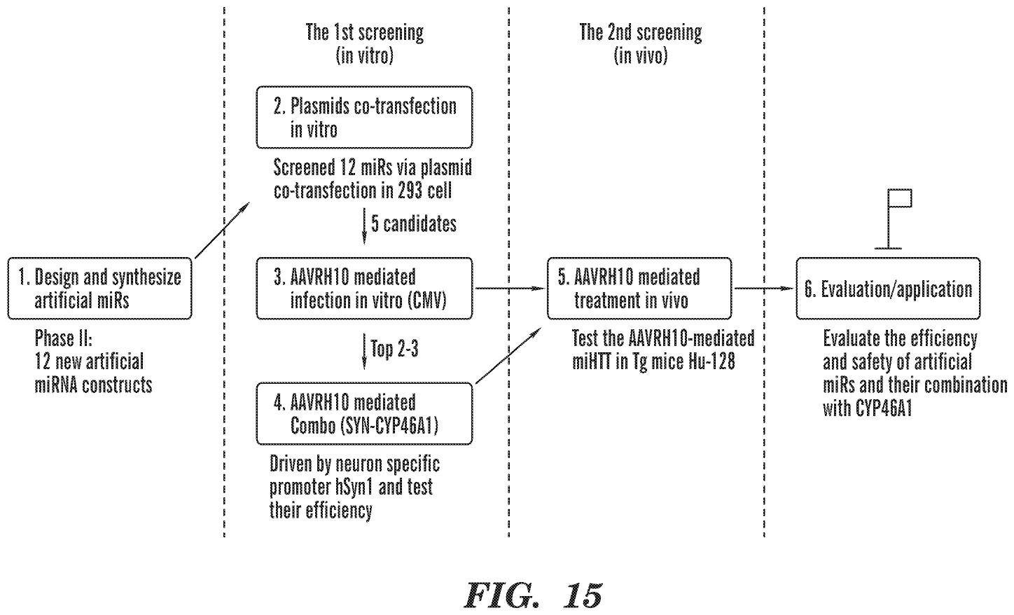

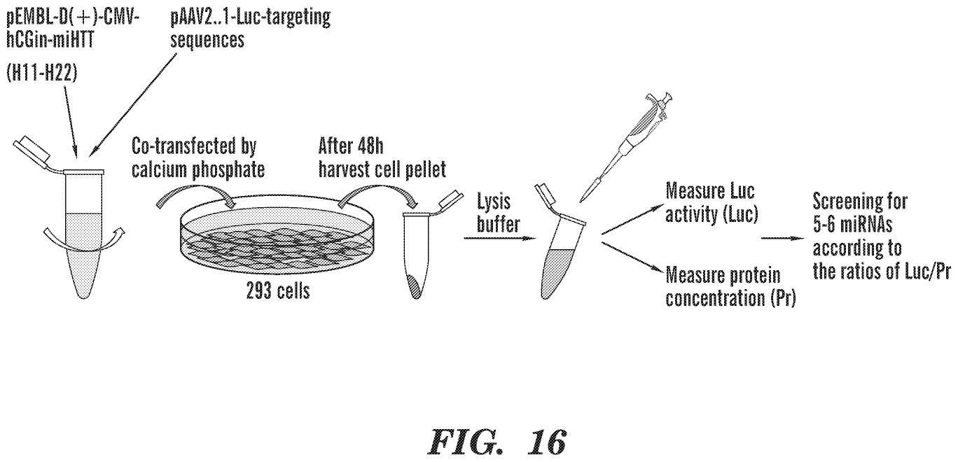

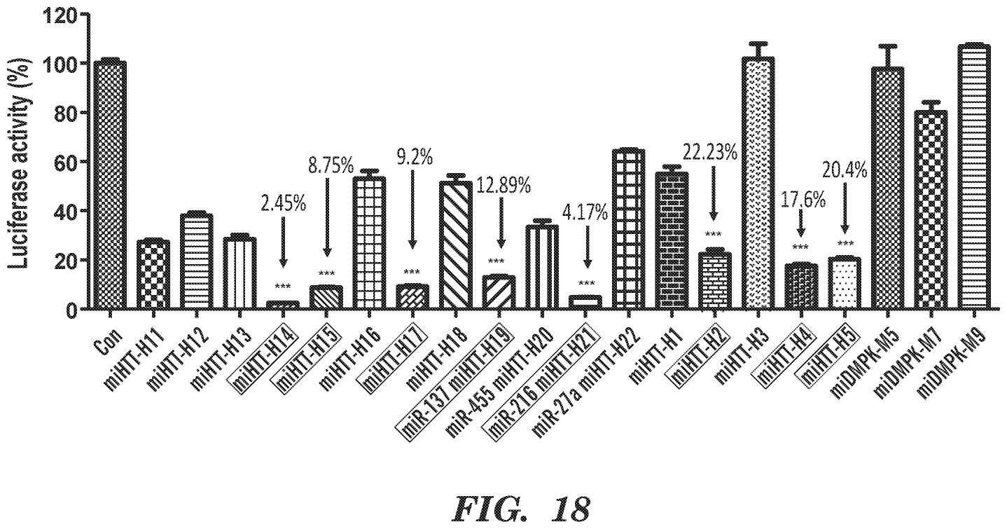

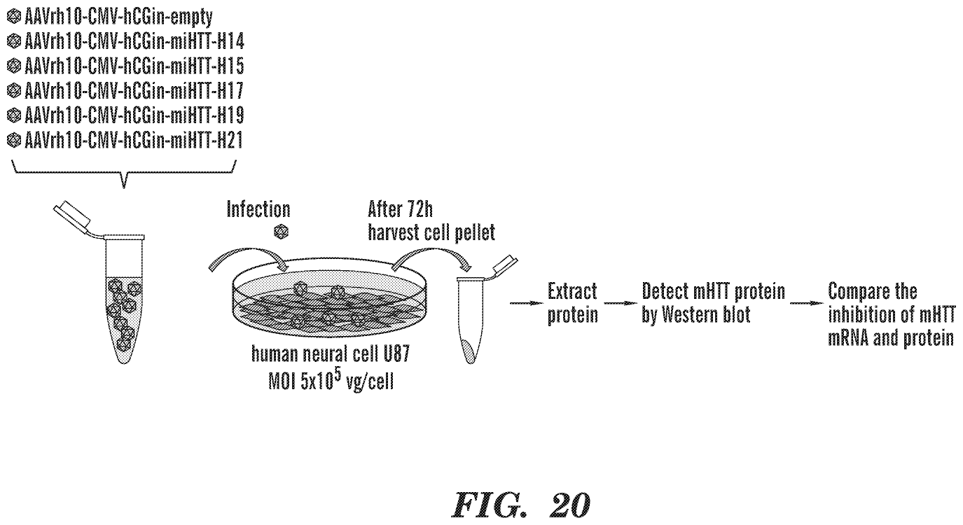

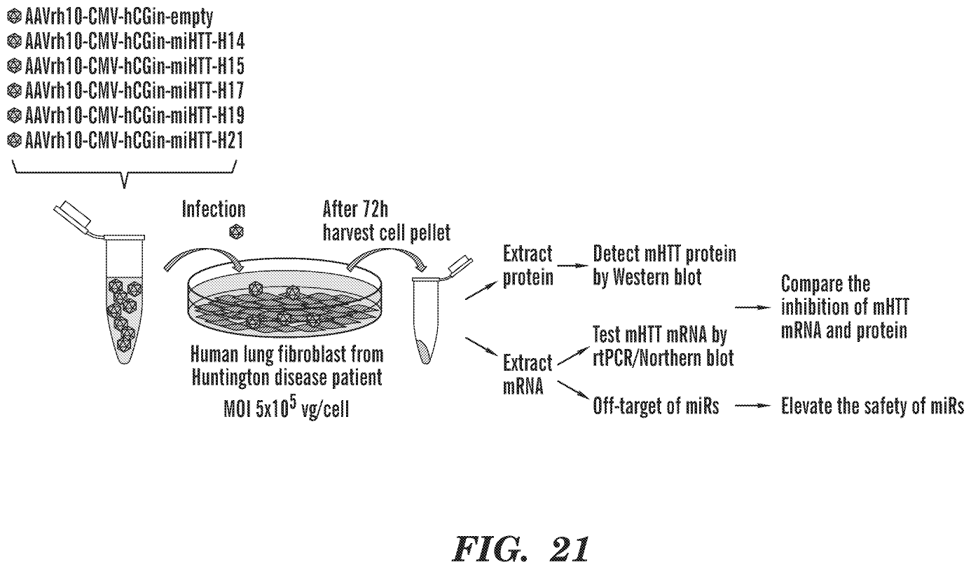

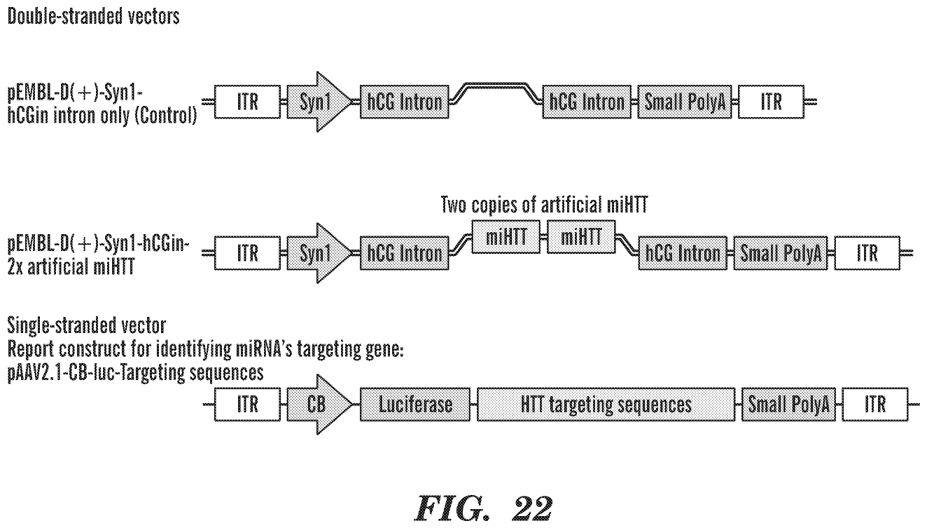

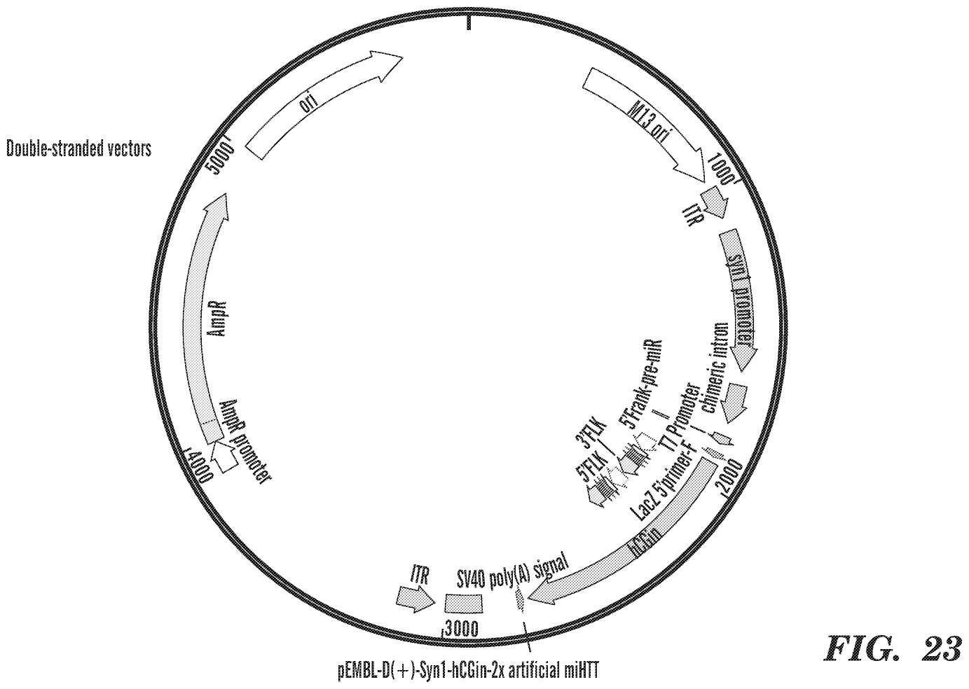

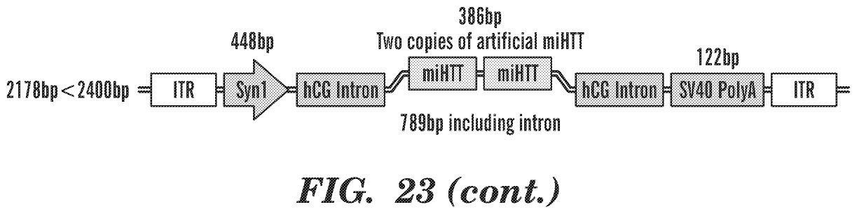

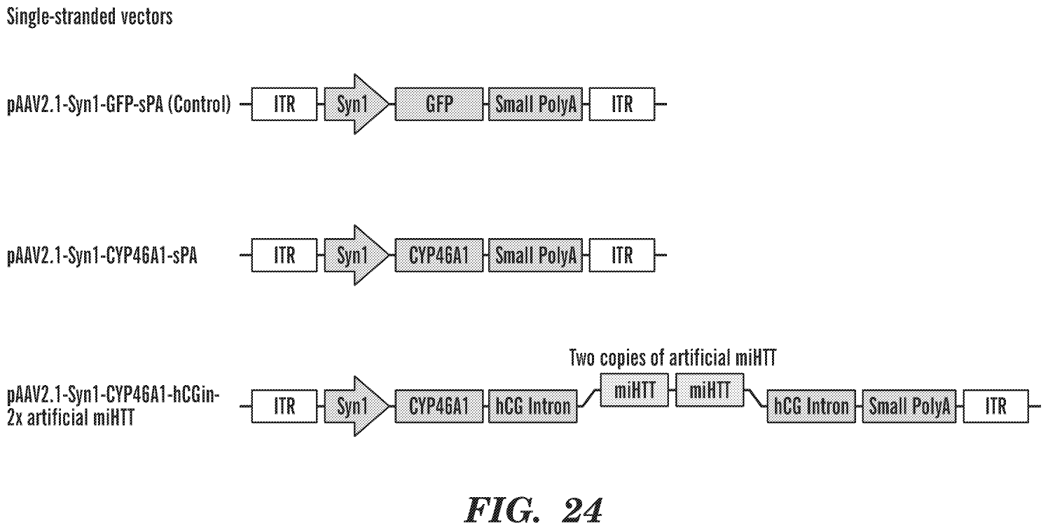

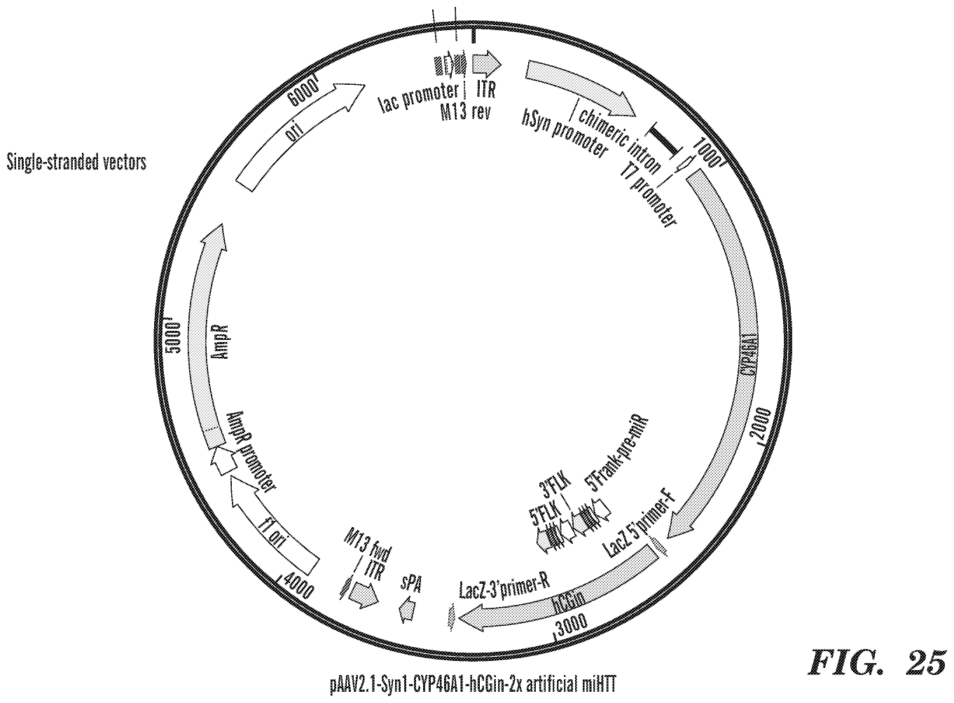

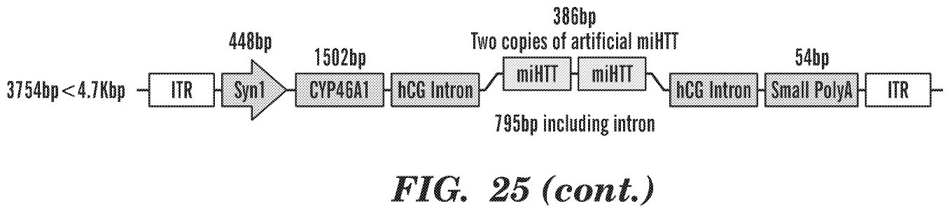

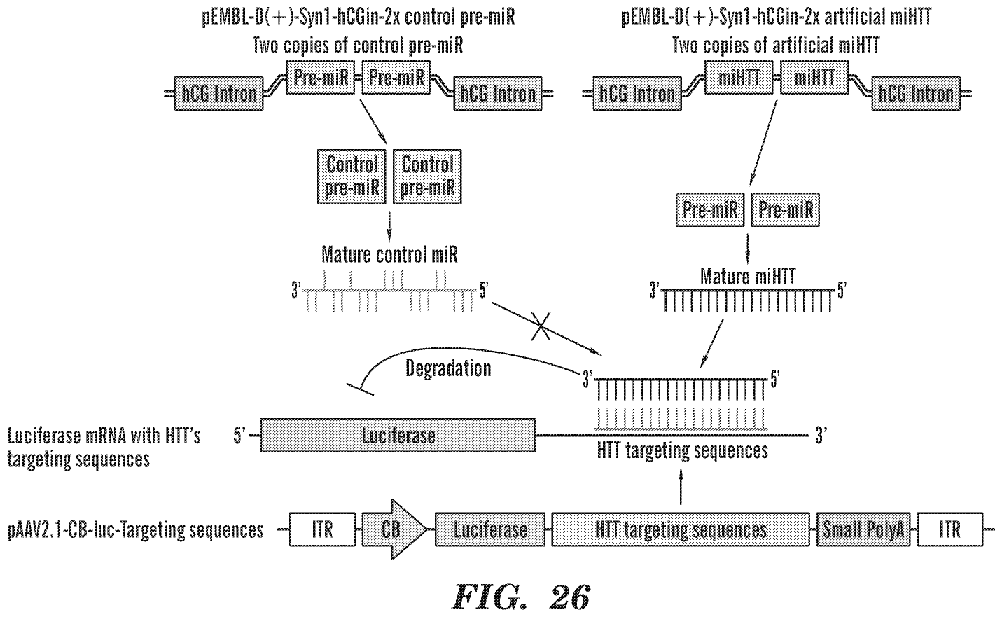

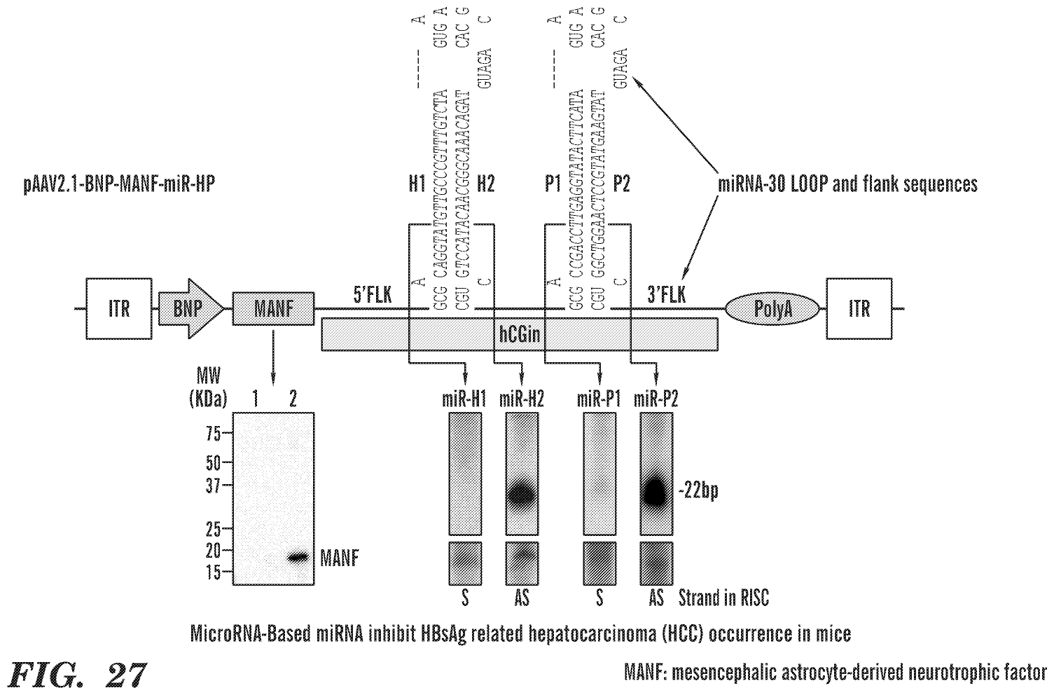

In vitro and animal model assays are provided herein which allow the assessment of a given dose of an isolated nucleic acid as described herein (e.g., at least one of SEQ ID NOs: 1-24) or a given dose of a recombinant AAV (rAAV) comprising an isolated nucleic acid as described herein (see e.g., FIG. 4, FIG. 9, FIG. 15. By way of non-limiting example, the efficacy of a dose of an isolated nucleic or an rAAV as described herein can be assessed in vitro by any one of the following methods: (1) co-transfecting isolated nucleic acids (e.g., a plasmid comprising at least one of SEQ ID NOs: 1-24) and a target sequence (e.g., HTT targeting sequences linked to luciferase expression) into cells (e.g., 293 cells) and measuring target concertation and/or activity (see e.g., FIG. 7, FIG. 8B, FIG. 16-18); (2) infecting cells (e.g., human neural cell U87) with rAAV (e.g., AAVRH10) expressing an isolated nucleic acid (e.g., at least one of SEQ ID NOs: 1-24; e.g., operably linked to a constitutive promoter, such as the CMV promoter) and rAAV expressing a target sequence (e.g., HTT targeting sequences linked to luciferase expression), and measuring target concertation and/or activity (see e.g., FIG. 10, 11A-11B); (3) infecting cells that express HTT (e.g., human neural cell U87 or a human lung fibroblast from a HD patient) with rAAV (e.g., AAVRH10) expressing an isolated nucleic acid (e.g., at least one of SEQ ID NOs: 1-24; e.g., operably linked to a constitutive promoter, such as the CMV promoter) and measuring HTT protein and/or mRNA concertation and/or activity (see e.g., FIG. 12-14, FIG. 20-21); or (4) infecting cells that express HTT (e.g., human neural cell U87 or a human lung fibroblast from a HD patient) with rAAV (e.g., AAVRH10) expressing an isolated nucleic acid (e.g., at least one of SEQ ID NOs: 1-24; e.g., operably linked to a neuron-specific promoter such as hSyn1, wherein the rAAV optionally further expresses a transgene encoding a protein, such as CYP46A1) and measuring HTT protein and/or mRNA concertation and/or activity (see e.g., FIG. 4, FIG. 9, FIG. 15).

The efficacy of an isolated nucleic or an rAAV as described herein can also be assessed in an animal model, e.g. a mouse model expressing HTT protein. For example, the following HTT mouse models can be used according to the desired HTT target (see e.g., Table 3): Hu128; B6CBA-R6/2 (CAG 120+/−5); B6CBA-Tg(HDexon1)62Gpb/3J; B6CBA-R6/2 (CAG 160+/−5); or B6CBA-Tg(HDexon1)62Gpb/1J. As a non-limiting example, a mouse model as described herein is infected with rAAV (e.g., AAVRH10) expressing an isolated nucleic acid (e.g., at least one of SEQ ID NOs: 1-24; e.g., operably linked to a neuron-specific promoter such as hSyn1, wherein the rAAV optionally further expresses a transgene encoding a protein, such as CYP46A1) and measuring HTT protein and/or mRNA concertation and/or activity, and/or disease pathogenesis in the mouse. See e.g., FIG. 4, FIG. 9, FIG. 15.

### Vectors

In some embodiments, one or more of the miRNAs described herein is expressed in a recombinant expression vector or plasmid. As used herein, the term “vector” refers to a polynucleotide sequence suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783, and 5,919,670, and Sambrook et al, Molecular Cloning: A Laboratory Manual, 2nd Ed., Cold Spring Harbor Press (1989). One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments, the vector is adeno-associated virus (AAV) or recombinant AAV.

In some embodiments of any of the aspects, the vector is pEMBL. In some embodiments of any of the aspects, the vector is pEMBL-D(+)Syn1. In some embodiments of any of the aspects, the vector is pEMBL-D(+)Syn1-hCG intron only. In some embodiments of any of the aspects, the vector is pEMBL-D(+)Syn1-hCGin-2×control pre-miR. In some embodiments of any of the aspects, the vector is pEMBL-D(+)Syn1-hCGin-2×artificial pre-miR. In some embodiments of any of the aspects, the vector is pEMBL-D(+)Syn1-CYP46A1-hCGin-2×artificial pre-miR. In some embodiments of any of the aspects, the vector is pEMBL-D(+)Syn1-luc-HTT-3′UTR/mutant.

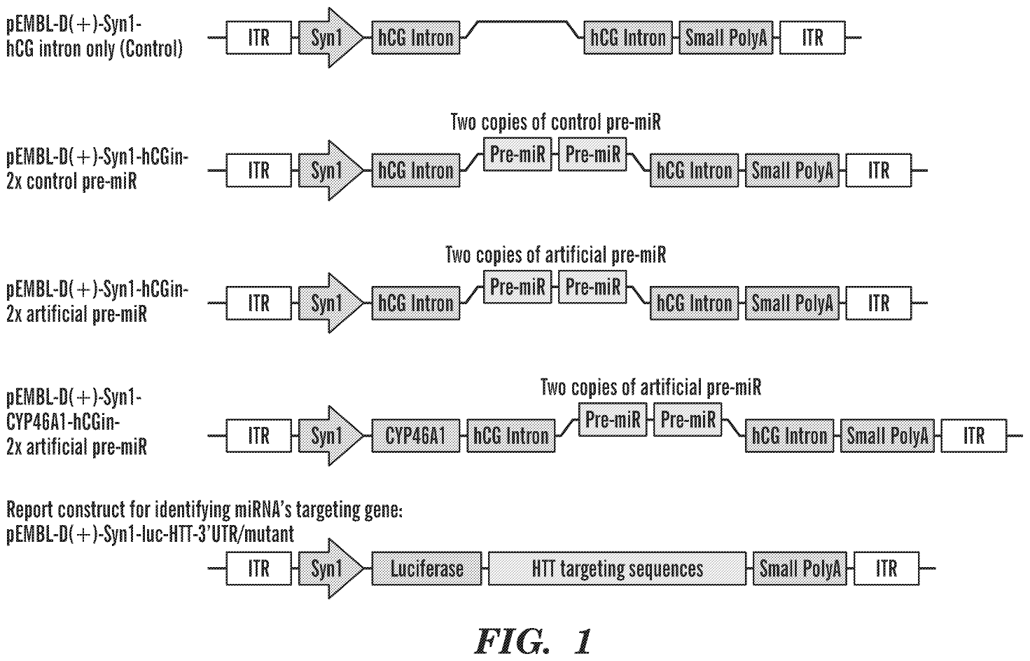

In some embodiments of any of the aspects, the vector or isolated nucleic as described herein comprises at least one of the following: at least one (e.g., 2) ITRs; Syn1 promoter (see e.g., SEQ ID NO: 31-32); at least one (e.g., 2) hCG intron (see e.g., SEQ ID NO: 34); at least one (e.g., 2) copy of a premiR (see e.g., SEQ ID NO: 35; e.g., control pre-miR; artificial pre-miR; at least one of SEQ ID NO: 1-24); small polyA (see e.g., SEQ ID NO: 36); CYP46A1 (see e.g., SEQ ID NO: 26-27); luciferase (see e.g., SEQ ID NO: 28-29); and/or HTT targeting sequences (see e.g., SEQ ID NO: 30) e.g., HTT-3′UTR/mutant). See e.g., FIG. 1.

In some embodiments of any of the aspects, an isolated nucleic acid or vector as described herein comprises CYP46A1. CYP46A1 is a member of the cytochrome P450 superfamily of enzymes. The cytochrome P450 proteins are monooxygenases which catalyze many reactions involved in drug metabolism and synthesis of cholesterol, steroids and other lipids. This endoplasmic reticulum protein is expressed in the brain, where it converts cholesterol to 24S-hydroxycholesterol. While cholesterol cannot pass the blood-brain barrier, 24S-hydroxycholesterol can be secreted in the brain into the circulation to be returned to the liver for catabolism. In some embodiments of any of the aspects, CYP46A1 can comprise a human CYP46A1 (see e.g., NCBI ref numbers NG_007963.1 RefSeqGene Range 4881-47884; NM_006668.2; NP_006659.1; see e.g., SEQ ID NO: 26-27). CYP46A1, the rate-limiting enzyme for cholesterol degradation, is neuroprotective in Huntington's disease (see e.g., Boussicault et al., CYP46A1, the rate-limiting enzyme for cholesterol degradation, is neuroprotective in Huntington's disease, Brain. 2016 March, 139(Pt 3):953-70; Kacher et al., CYP46A1 gene therapy deciphers the role of brain cholesterol metabolism in Huntington's disease, Brain. 2019 Aug. 1; 142(8):2432-2450; the contents of each of which are incorporated herein by reference in their entireties).

In some embodiments of any of the aspects, the transgene as described herein (e.g., CYP46A1) comprises SEQ ID NO: 27, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 27, that maintains the same function as SEQ ID NO: 27 (e.g., therapeutic protein for HD).

In some embodiments of any of the aspects, the transgene as described herein (e.g., CYP46A1) is encoded by a nucleic acid sequence comprising SEQ ID NO: 26 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to SEQ ID NO: 26 that maintains the same function or a codon-optimized version of SEQ ID NO: 26.

SEQ ID NO: 26, CYP46A1, 1503 nucleotides (nt) (see e.g., Homo sapiens cytochrome
P450 family 46 subfamily A member 1 (CYP46A1), mRNA, NCBI Reference Sequence:
NM_006668.2)
tgagccccgggctgctgctgctcggcagcgccgtcctgctcgccttcggcctctgctgcaccttcgtgcaccgcgctcgcagccgctacgagcac
atccccgggccgccgcggcccagtttccttctaggacacctcccctgcttttggaaaaaggatgaggttggtggccgtgtgctccaagatgtgttttt
ggattgggctaagaagtatggacctgttgtgcgggtcaacgtcttccacaaaacctcagtcatcgtcacgagtcctgagtcggttaagaagttcctga
tgtcaaccaagtacaacaaggactccaagatgtaccgtgcgctccagactgtgtttggtgagagactcttcggccaaggcttggtgtccgaatgcaa
ctatgagcgctggcacaagcageggagagtcatagacctggccttcagccggagctccttggttagcttaatggaaacattcaacgagaaggctg
agcagctggtggagattctagaagccaaggcagatgggcagaccccagtgtccatgcaggacatgctgacctacaccgccatggacatcctggc
caaggcagcttttgggatggagaccagtatgctgctgggtgcccagaagcctctgtcccaggcagtgaaacttatgttggagggaatcactgcgtc
ccgcaacactctggcaaagttcctgccagggaagaggaagcagctccgggaggtccgggagagcattcgcttcctgcgccaggtgggcaggg
actgggtccagcgccgccgggaagccctgaagaggggcgaggaggttcctgccgacatcctcacacagattctgaaagctgaagagggagcc
caggacgacgagggtctgctggacaacttcgtcaccttcttcattgctggtcacgagacctctgccaaccacttggcgttcacagtgatggagctgtc
tcgccagccagagatcgtggcaaggctgcaggccgaggtggatgaggtcattggttctaagaggtacctggatttcgaggacctggggagactg
cagtacctgtcccaggtcctcaaagagtcgctgaggctgtacccaccagcatggggcacctttcgcctgctggaagaggagaccttgattgatggg
gtcagagtccccggcaacaccccgctcttgttcagcacctatgtcatggggcggatggacacatactttgaggacccgctgactttcaaccccgatc
gcttcggccctggagcacccaagccacggttcacctacttccccttctccctgggccaccgctcctgcatcgggcagcagtttgctcagatggaggt
gaaggtggtcatggcaaagctgctgcagaggctggagttccggctggtgcccgggcagcgcttcgggctgcaggagcaggccacactcaagcc
actggaccccgtgctgtgcaccctgcggccccgcggctggcagcccgcacccccaccacccccctgctga
SEQ ID NO: 27, CYP46A1, 500 amino acids (aa) (see e.g., cholesterol 24-hydroxylase
precursor (Homo sapiens), NCBI Reference Sequence: NP_006659.1)
MSPGLLLLGSAVLLAFGLCCTFVHRARSRYEHIPGPPRPSFLLGHLPCFWKKDEVGG
RVLQDVFLDWAKKYGPVVRVNVFHKTSVIVTSPESVKKFLMSTKYNKDSKMYRALQTVFG
MQDMLTYTAMDILAKAAFGMETSMLLGAQKPLSQAVKLMLEGITASRNTLAKFLPGKRKQ
LREVRESIRFLRQVGRDWVQRRREALKRGEEVPADILTQILKAEEGAQDDEGLLDNFVTFFIA
GHETSANHLAFTVMELSRQPEIVARLQAEVDEVIGSKRYLDFEDLGRLQYLSQVLKESLRLYP
PAWGTFRLLEEETLIDGVRVPGNTPLLFSTYVMGRMDTYFEDPLTFNPDRFGPGAPKPRFTYF
PFSLGHRSCIGQQFAQMEVKVVMAKLLQRLEFRLVPGQRFGLQEQATLKPLDPVLCTLRPRG
WQPAPPPPPC

In some embodiments, one or more of the recombinantly expressed gene can be integrated into the genome of the cell.

In some embodiments of any of the aspects, an isolated nucleic acid or vector as described herein comprises at least one promoter. In some embodiments of any of the aspects, the promoter is human Syn1 promoter. In some embodiments of any of the aspects, the promoter as described herein (e.g., Syn1) comprises one of SEQ ID NOs: 31-32, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 31-32, that maintains the same function as one of SEQ ID NOs: 31-32 (e.g., tissue-specific promoter).

SEQ ID NO: 31, Syn1 promoter, 477 nt
agtgcaagtgggttttaggaccaggatgaggcggggtgggggtgcctacctgacgaccgaccccgacccactggacaagcacccaacccccat
tccccaaattgcgcatcccctatcagagagggggaggggaaacaggatgcggcgaggcgcgtgcgcactgccagcttcagcaccgcggacag
tgccttcgcccccgcctggcggcgcgcgccaccgccgcctcagcactgaagggcgctgacgtcactcgccggtcccccgcaaactccccttcc
cggccaccttggtcgcgtccgcgccgccgccggcccagccggaccgcaccacgcgaggcgcgagataggggggcacgggcgcgaccatct
gcgctgcggcgccggcgactcagcgctgcctcagtctgcggtgggcagcggaggagtcgtgtcgtgcctgagagcgcagtcgaggcgcgccg
agctcggatcctgacg
SEQ ID NO: 32, Syn1 promoter, 448 nt
AGTGCAAGTGGGTTTTAGGACCAGGATGAGGCGGGGTGGGGGTGCCTACCTGACGACCG
ACCCCGACCCACTGGACAAGCACCCAACCCCCATTCCCCAAATTGCGCATCCCCTATCAG
AGAGGGGGAGGGGAAACAGGATGCGGCGAGGCGCGTGCGCACTGCCAGCTTCAGCACC
GCGGACAGTGCCTTCGCCCCCGCCTGGCGGCGCGCGCCACCGCCGCCTCAGCACTGAAG
GCGCGCTGACGTCACTCGCCGGTCCCCCGCAAACTCCCCTTCCCGGCCACCTTGGTCGCG
TCCGCGCCGCCGCCGGCCCAGCCGGACCGCACCACGCGAGGCGCGAGATAGGGGGGCAC
GGGCGCGACCATCTGCGCTGCGGCGCCGGCGACTCAGCGCTGCCTCAGTCTGCGGTGGG
CAGCGGAGGAGTCGTGTCGTGCCTGAGAGCGCAG

In some embodiments of any of the aspects, the promoter is the constitutive CMV promoter. In some embodiments of any of the aspects, the promoter as described herein (e.g., CMV) comprises SEQ ID NO: 33, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 33, that maintains the same function as SEQ ID NO: 33 (e.g., constitutive promoter).

SEQ ID NO: 33, CMV promoter
GTGATGCGGTTTTGGCAGTACACCAATGGGCGTGGATAGCGGTTTGACTC
ACGGGGATTTCCAAGTCTCCACCCCATTGACGTCAATGGGAGTTTGTTTT
GGCACCAAAATCAACGGGACTTTCCAAAATGTCGTAATAACCCCGCCCCG
TTGACGCAAATGGGCGGTAGGCGTGTACGGTGGGAGGTCTATATAAGCAG
AGCT

In some embodiments of any of the aspects, an isolated nucleic acid or vector as described herein comprises at least one intron. In some embodiments of any of the aspects, the intron is the hCG intron. In some embodiments of any of the aspects, the intron as described herein (e.g., hCG intron) comprises SEQ ID NO: 34, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 34, that maintains the same function as SEQ ID NO: 34. In some embodiments of any of the aspects, an isolated nucleic acid or vector as described herein comprises at least one (e.g., 2) portion(s) of SEQ ID NO: 34, e.g., approximately nucleotides 1-100, 16-100, 114-635, and/or 122-635, of SEQ ID NO: 34.

SEQ ID NO: 34, hCG intron, 635 nt (see e.g., Human
chorionic gonadotropin (HCG) gene 6 beta subunit,
GenBank: X00266.1)
CTAGCACATCGATACGGTACCCACCGATATTATTTGCCCGATGGTATCC
CCGTTTACAGGTAAGAAGATCTGGCGCGCCTCACTAGTACCTCGAGATT
ACGAAGATATCTTACCTGAGTCGACACCCTAGGACAGATCTTCCGGACT
GGGCACCTTCCACCTCCTTCCAGGCAATCACTGGCATGAGAAGGGGCAG
ACCAGTGTGAGCTGTGGAAGGACGCCTCTTTCTGGAGGAGTGTGACCCC
CAGTAAGCTTCACGTGGGGCAGTTCCTGAGGGTGGGGATCTGAAATGTT
GGGGTATCTCAGGTCCCTCGGGCTGTGGGGTGGGCTCTGAAAGGCAGGT
GTCCGGGTGGTGGGTCCTGAATAGGAGATGCCGGGAAGGGTCTCTGGGT
CTTTGTGGGTGGTGTACCCTGGGGGATGGGAAGGCCGGGGCTCAGGGCT
GTGGTCTCAGGCCCGGGTGAAGCAGTGTCCTTGTCCGGTTACCCTGCAG
GGCGGCTTCGTCTGGGTTCCGTTTATCCGGGCAAACCGGGCCCGCGACT
CTAGATCATAATCAGCCATACCACATTTGTAGAGGTTTTACTTGCTTTA
AAAAACCTCCCACACCTCCCCCTGAACCTGAAACATAAAATGAATGC

In some embodiments of any of the aspects, an isolated nucleic acid or vector as described herein comprises a premiR comprising two copies of an artificial miRNA (e.g., one of SEQ ID NOs: 1-24) as described herein. In some embodiments of any of the aspects, the premiR as described herein comprises SEQ ID NO: 35, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 35, that maintains the same function as SEQ ID NO: 35 (e.g., expression of at least one miRNA). In some embodiments of any of the aspects, isolated nucleic acid or vector as described herein comprises a portion of SEQ ID NO: 35, e.g., such that one miRNA copy is expressed, e.g., approximately nt 1-191, 1-199, 191-386, or 199-386 of SEQ ID NO: 35.

SEQ ID NO: 35, 2×miHTT-H1; bolded text indicates the 5′ flank sequence (e.g., nt 1-65 or 199-260 of SEQ ID NO: 35); italicized text indicates the miHTT passenger (e.g., nt 69-87 or 264-282 of SEQ ID NO: 35; see e.g., the reverse complement of SEQ ID NO: 1, which is used as a non-limiting example; the reverse complement of any one of SEQ ID NOs: 2-24 can be used in the place of the reverse complement of SEQ ID NO: 1 in SEQ ID NO: 35); bold italicized text indicates the miR30a loop (e.g., nt 90-104 or 285-299 of SEQ ID NO: 35); italicized double underlined text indicates the miHTT guide strand (e.g., nt 107-126 or 302-321 of SEQ ID NO: 35; see e.g., SEQ ID NO: 1, which is used as a non-limiting example; any one of SEQ ID NOs: 2-24 can be used in the place of SEQ ID NO: 1 in SEQ ID NO: 35); and bolded double underlined text indicates the 3′ flank sequence (e.g., nt 130-191 or 325-386 of SEQ ID NO: 35).

agattacttcttcaggttaacccaacagaaggctaaagaaggtatattgctgttgacagtgagcgacgCAGCAGCAGCAGCAG
CAGCtagtgaagccacagatgtaGcTGcTGCTGCTGcTGCTGCgctgcctactgcctcggacttcaaggggctactttagga
gcaattatcttgtttactaaaactgaagatatcttacttcttcaggttaacccaacagaaggctaaagaaggtatattgctgttgacagtgagc
gacgCAGCAGCAGCAGCAGCAGCta [symbol] taGcTGcTGCTGCTGcTGCTGCgctgcctactgcc
tcggacttcaaggggctactttaggagcaattatcttgtttactaaaactga

In some embodiments of any of the aspects, an isolated nucleic acid or vector as described herein comprises a polyadenylation region (e.g., small polyA). In some embodiments of any of the aspects, the polyA as described herein comprises SEQ ID NO: 36, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 36, that maintains the same function as SEQ ID NO: 36 (e.g., polyadenylation).

SEQ ID NO: 36, small polyA, 54 nt,
aggcctaataaagagctcagatgcatcgatcagagtgtgttggttttttg
tgtg

In some embodiments of any of the aspects, an isolated nucleic acid or vector as described herein (e.g., used to test the miRNAs as described herein) comprises luciferase as a reporter. In some embodiments of any of the aspects, the reporter as described herein (e.g., luciferase) comprises SEQ ID NO: 29, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 29, that maintains the same function as SEQ ID NO: 29 (e.g., luciferase activity and luminescence).

In some embodiments of any of the aspects, the reporter as described herein (e.g., luciferase) is encoded by a nucleic acid sequence comprising SEQ ID NO: 28 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to SEQ ID NO: 28 that maintains the same function or a codon-optimized version of SEQ ID NO: 28.

SEQ ID NO: 28, luciferase nucleic acid, 1653 nt
ATGGAAGACGCCAAAAACATAAAGAAAGGCCCGGCGCCATTCTATCCGCTGGAAGATGG
AACCGCTGGAGAGCAACTGCATAAGGCTATGAAGAGATACGCCCTGGTTCCTGGAACAA
TTGCTTTTACAGATGCACATATCGAGGTGGACATCACTTACGCTGAGTACTTCGAAATGT
CCGTTCGGTTGGCAGAAGCTATGAAACGATATGGGCTGAATACAAATCACAGAATCGTC
GTATGCAGTGAAAACTCTCTTCAATTCTTTATGCCGGTGTTGGGCGCGTTATTTATCGGAG
TTGCAGTTGCGCCCGCGAACGACATTTATAATGAACGTGAATTGCTCAACAGTATGGGCA
TTTCGCAGCCTACCGTGGTGTTCGTTTCCAAAAAGGGGTTGCAAAAAATTTTGAACGTGC
AAAAAAAGCTCCCAATCATCCAAAAAATTATTATCATGGATTCTAAAACGGATTACCAG
GGATTTCAGTCGATGTACACGTTCGTCACATCTCATCTACCTCCCGGTTTTAATGAATACG
ATTTTGTGCCAGAGTCCTTCGATAGGGACAAGACAATTGCACTGATCATGAACTCCTCTG
GATCTACTGGTCTGCCTAAAGGTGTCGCTCTGCCTCATAGAACTGCCTGCGTGAGATTCT
CGCATGCCAGAGATCCTATTTTTGGCAATCAAATCATTCCGGATACTGCGATTTTAAGTG
TTGTTCCATTCCATCACGGTTTTGGAATGTTTACTACACTCGGATATTTGATATGTGGATT
TCGAGTCGTCTTAATGTATAGATTTGAAGAAGAGCTGTTTCTGAGGAGCCTTCAGGATTA
CAAGATTCAAAGTGCGCTGCTGGTGCCAACCCTATTCTCCTTCTTCGCCAAAAGCACTCT
GATTGACAAATACGATTTATCTAATTTACACGAAATTGCTTCTGGTGGCGCTCCCCTCTCT
AAGGAAGTCGGGGAAGCGGTTGCCAAGAGGTTCCATCTGCCAGGTATCAGGCAAGGATA
TGGGCTCACTGAGACTACATCAGCTATTCTGATTACACCCGAGGGGGATGATAAACCGG
GCGCGGTCGGTAAAGTTGTTCCATTTTTTGAAGCGAAGGTTGTGGATCTGGATACCGGGA
AAACGCTGGGCGTTAATCAAAGAGGCGAACTGTGTGTGAGAGGTCCTATGATTATGTCC
GGTTATGTAAACAATCCGGAAGCGACCAACGCCTTGATTGACAAGGATGGATGGCTACA
TTCTGGAGACATAGCTTACTGGGACGAAGACGAACACTTCTTCATCGTTGACCGCCTGAA
GTCTCTGATTAAGTACAAAGGCTATCAGGTGGCTCCCGCTGAATTGGAATCCATCTTGCT
CCAACACCCCAACATCTTCGACGCAGGTGTCGCAGGTCTTCCCGACGATGACGCCGGTGA
ACTTCCCGCCGCCGTTGTTGTTTTGGAGCACGGAAAGACGATGACGGAAAAAGAGATCG
TGGATTACGTCGCCAGTCAAGTAACAACCGCGAAAAAGTTGCGCGGAGGAGTTGTGTTT
GTGGACGAAGTACCGAAAGGTCTTACCGGAAAACTCGACGCAAGAAAAATCAGAGAGA
TCCTCATAAAGGCCAAGAAGGGCGGAAAGATCGCCGTGTAA
SEQ ID NO: 29, luciferase protein, 550 aa
MEDAKNIKKGPAPFYPLEDGTAGEQLHKAMKRYALVPGTIAFTDAHIEVDITYAEYFEMSVR
LAEAMKRYGLNTNHRIVVCSENSLQFFMPVLGALFIGVAVAPANDIYNERELLNSMGISQPT
VVFVSKKGLQKILNVQKKLPIIQKIIIMDSKTDYQGFQSMYTFVTSHLPPGFNEYDFVPESFDR
DKTIALIMNSSGSTGLPKGVALPHRTACVRFSHARDPIFGNQIIPDTAILSVVPFHHGFGMFTTL
GYLICGFRVVLMYRFEEELFLRSLQDYKIQSALLVPTLFSFFAKSTLIDKYDLSNLHEIASGGA
PLSKEVGEAVAKRFHLPGIRQGYGLTETTSAILITPEGDDKPGAVGKVVPFFEAKVVDLDTGK
TLGVNQRGELCVRGPMIMSGYVNNPEATNALIDKDGWLHSGDIAYWDEDEHFFIVDRLKSLI
KYKGYQVAPAELESILLQHPNIFDAGVAGLPDDDAGELPAAVVVLEHGKTMTEKEIVDYVA
SQVTTAKKLRGGVVFVDEVPKGLTGKLDARKIREILIKAKKGGKIAV

In some embodiments of any of the aspects, an isolated nucleic acid or vector as described herein (e.g., used to test the miRNAs as described herein) comprises an HTT targeting sequence (i.e., short nucleic acid sequences from the HTT gene). In some embodiments of any of the aspects, the HTT targeting sequence as described herein comprises SEQ ID NO: 30, or a nucleic acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 30, that maintains the same function as SEQ ID NO: 30 (e.g., targeting or testing of miRNAs as described herein).

SEQ ID NO: 30, HTT targeting seq (e.g.,
HTT-3′UTR/mutant), 417 nt
acgcagcagcagcagcagcagcacagcagcagcagcagcagcagaccagc
agcagcagcagcagacccgccgccgccgccgccgccactccctcaagtcc
ttccagcaaccagcagcagcagcagcaacaactgtgccccgccccggcct
cgacccctccacggccccgccccgactgaaaacatagtggcacagtacct
ggaaaagctgatgaaggcacccccagcctccccacccctcacattttaat
gaaaccagggtaacttatatcagtaaagagattaaccctaggacagatct
actccggaacgagctcacgactctagatcataatcagccataccacattt
gtagaggttttacttgctttaaaaaacctcccacacctccccctgaacct
gaaacataaaatgaatg

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

### Kits

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the instant disclosure relates to a kit for producing a rAAV, the kit comprising a container housing an isolated nucleic acid comprising an miRNA comprising or encoded by the sequence set forth in any one of SEQ ID NOs: 1-24. In some embodiments, the kit further comprises a container housing an isolated nucleic acid encoding an AAV capsid protein, for example an AAV9 or an AAVrh10 capsid protein.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container.

Exemplary embodiments of the invention will be described in more detail by the following examples. These embodiments are exemplary of the invention, which one skilled in the art will recognize is not limited to the exemplary embodiments.

### Immune Modulators

In some embodiments, the methods and compositions described herein, further comprise administering an immune modulator. In some embodiments, the immune modulator can be administered at the time of administration, before administration or, after the administration. In the case in which a subject is re-administered at least a second composition, the immune modulator can be administered prior to, with, or after the at least second administration.

In some embodiments, the immune modulator is an immunoglobulin degrading enzyme such as IdeS, IdeZ, IdeS/Z, Endo S, or, their functional variant. Non-limiting examples of references of such immunoglobulin degrading enzymes and their uses as described in U.S. Pat. Nos. 7,666,582, 8,133,483, US 20180037962, US 20180023070, US 20170209550, U.S. Pat. No. 8,889,128, WO2010/057626, U.S. Pat. Nos. 9,707,279, 8,323,908, US 20190345533, US 20190262434, and WO2020/016318, each of which are incorporated in their entirety by reference.

In some embodiments, the immune modulator is Proteasome inhibitor. In certain aspects, the proteasome inhibitor is Bortezomib. In some aspects of the embodiment, the immune modulator comprises bortezomib and anti CD20 antibody, Rituximab. In other aspects of the embodiment, the immune modulator comprises bortezomib, Rituximab, methotrexate, and intravenous gamma globulin. Non-limiting examples of such references, disclosing proteasome inhibitors and their combination with Rituximab, methotrexate and intravenous gamma globulin, as described in U.S. Pat. Nos. 10,028,993, 9,592,247, and 8,809,282, each of which are incorporated in their entirety by reference.

In alternative embodiments, the immune modulator is an inhibitor of the NF-kB pathway. In certain aspects of the embodiment, the immune modulator is Rapamycin or, a functional variant. Non-limiting examples of references disclosing rapamycin and its use described in U.S. Pat. No. 10,071,114, US 20160067228, US 20160074531, US 20160074532, US 20190076458, U.S. Pat. No. 10,046,064, are incorporated in their entirety. In other aspects of the embodiment, the immune modulator is synthetic nanocarriers comprising an immunosuppressant. Non limiting examples of references of immunosuppressants, immunosuppressants coupled to synthetic nanocarriers, synthetic nanocarriers comprising rapamycin, and/or, tolerogenic synthetic nanocarriers, their doses, administration and use as described in US20150320728, US 20180193482, US 20190142974, US 20150328333, US20160243253, U.S. Pat. No. 10,039,822, US 20190076522, US 20160022650, U.S. Pat. Nos. 10,441,651, 10,420,835, US 20150320870, US 2014035636, U.S. Pat. Nos. 10,434,088, 10,335,395, US 20200069659, U.S. Pat. No. 10,357,483, US 20140335186, U.S. Pat. Nos. 10,668,053, 10,357,482, US 20160128986, US 20160128987, US 20200038462, US 20200038463, each of which are incorporated in their entirety by reference.

In some embodiments, the immune modulator is synthetic nanocarriers comprising rapamycin (ImmTOR™ nanoparticles) (Kishimoto, et al., 2016, Nat Nanotechnol, 11(10): 890-899; Maldonado, et al., 2015, PNAS, 112(2): E156-165), as disclosed in US20200038463, U.S. Pat. No. 9,006,254 each of which is incorporated herein in its entirety. In some embodiments, the immune modulator is an engineered cell, e.g., an immune cell that has been modified using SQZ technology as disclosed in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In another further embodiment, the immunomodulator or adjuvant is poly-ICLC

In some embodiments, the immune modulator is a small molecule that inhibit the innate immune response in cells, such as chloroquine (a TLR signaling inhibitor) and 2-aminopurine (a PKR inhibitor), can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN™). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, a rAAV vector having the modified viral capsid can also encode a negative regulators of innate immunity such as NLRX1. Accordingly, in some embodiments, a rAAV vector can also optionally encode one or more, or any combination of NLRX1, NS1, NS3/4A, or A46R. Additionally, in some embodiments, a composition comprising at least one rAAV vector as disclosed herein can also comprise a synthetic, modified-RNA encoding inhibitors of the innate immune system to avoid the innate immune response generated by the tissue or the subject.

In some embodiments, an immune modulator for use in the administration methods as disclosed herein is an immunosuppressive agent. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211. In one embodiment, an immunosuppressive agent is cyclosporine A. Other examples include myophenylate mofetil, rapamicin, and anti-thymocyte globulin. In one embodiment, the immunosuppressive drug is administered in a composition comprising at least one rAAV vector as disclosed herein, or can be administered in a separate composition but simultaneously with, or before or after administration of a composition comprising at least one rAAV vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

In any embodiment of the methods and compositions as disclosed herein, a subject being administered a composition disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In some embodiments, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

The use of such immune modulating agents facilitates the ability to for one to use multiple dosing (e.g., multiple administration) over numerous months and/or years. This permits using multiple agents as discussed below, e.g., a rAAV vector encoding multiple genes, or multiple administrations to the subject.

Exemplary embodiments of the invention will be described in more detail by the following examples. These embodiments are exemplary of the invention, which one skilled in the art will recognize is not limited to the exemplary embodiments.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of Huntington's disease. A subject can be male or female.

A subject can be one who has been previously diagnosed with or identified as suffering from or having a condition in need of treatment (e.g. Huntington's disease) or one or more complications related to such a condition, and optionally, have already undergone treatment for Huntington's disease or the one or more complications related to Huntington's disease. Alternatively, a subject can also be one who has not been previously diagnosed as having Huntington's disease or one or more complications related to Huntington's disease. For example, a subject can be one who exhibits one or more risk factors for Huntington's disease or one or more complications related to Huntington's disease or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA, miRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the miRNA described herein is exogenous.

In some embodiments of any of the aspects, the miRNA described herein is ectopic. In some embodiments of any of the aspects, the miRNA described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments, a nucleic acid encoding an inhibitory RNA as described herein (e.g. SEQ ID NO: 1-24) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Non-limiting examples of a viral vector of this invention include an AAV vector, an adenovirus vector, a lentivirus vector, a retrovirus vector, a herpesvirus vector, an alphavirus vector, a poxvirus vector a baculovirus vector, and a chimeric virus vector.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. Huntington's disease. The term “treating” includes reducing or alleviating at least one adverse effect or symptom of a condition, disease or disorder associated with Huntington's disease. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), WO 2018/057855A, U.S. Pat. No. 10,457,940, the contents of each of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. An isolated nucleic acid comprising:
   a. a first region comprising a first adeno-associated virus (AAV) inverted terminal repeat (ITR), or a variant thereof; and
   b. a second region comprising a transgene encoding one or more miRNAs, wherein each miRNA comprises a seed sequence complementary to SEQ ID NO: 25.
2. An isolated nucleic acid comprising:
   a. a first region comprising a first adeno-associated virus (AAV) inverted terminal repeat (ITR), or a variant thereof; and
   b. a second region comprising a transgene encoding one or more miRNAs, wherein each miRNA is encoded by a sequence comprising the sequence set forth in any one of SEQ ID NOs: 1-22 flanked by a miRNA backbone sequence.
3. The isolated nucleic acid of paragraphs 1 or 2, wherein the transgene comprises two miRNA or two precursor miRNAs in tandem that are flanked by introns.
4. The isolated nucleic acid of paragraph 3, wherein the flanking introns are identical.
5. The isolated nucleic acid of paragraph 3, wherein the flanking introns are from the same species.
6. The isolated nucleic acid of paragraph 3, wherein the flanking introns are hCG introns.
7. The isolated nucleic acid of any one of paragraphs 1 to 6, wherein the transgene comprises a promoter.
8. The isolated nucleic acid of paragraph 7, wherein the promoter is a synapsin (Syn1) promoter.
9. The isolated nucleic acid of any one of paragraphs 1 to 8, wherein the transgene further encodes a protein.
10. The isolated nucleic acid of paragraph 9, wherein the protein is CYP46A1.
11. The isolated nucleic acid of any one of paragraphs 1 to 10, wherein the one or more miRNAs are located in an untranslated portion of the transgene.
12. The isolated nucleic acid of paragraph 11, wherein the untranslated portion is an intron.
13. The isolated nucleic acid of paragraph 11, wherein the untranslated portion is between the last codon of the nucleic acid sequence encoding a protein and a poly-A tail sequence, or between the last nucleotide base of a promoter sequence and a poly-A tail sequence.
14. The isolated nucleic acid of any one of paragraphs 1 to 13 further comprising a third region comprising a second adeno-associated virus (AAV) inverted terminal repeat (ITR), or a variant thereof.
15. The isolated nucleic acid of any one of paragraphs 1 to 14, wherein the ITR variant lacks a functional terminal resolution site (TRS), optionally wherein the ITR variant is a ATRS ITR.
16. The isolated nucleic acid of any one of paragraphs 1 to 15, wherein at least one of the miRNAs hybridizes with and inhibits expression of human huntingtin (e.g., SEQ ID NO: 25).
17. A vector comprising the isolated nucleic acid of any one of paragraphs 1 to 16.
18. The vector of paragraph 17, wherein the vector is a plasmid.
19. A host cell comprising the isolated nucleic acid of any one of paragraphs 1 to 16, or the vector of paragraph 17 or 18.
20. A recombinant AAV (rAAV) comprising:
   a. a capsid protein; and
   b. the isolated nucleic acid of any one of paragraphs 1 to 16.
21. The rAAV of paragraph 20, wherein the capsid protein is an AAV1, AAV2, AAV3b, AAV4, AAV5, AAV6, AAV7, AAV8, AAV9, AAV10, AAV11, AAV12, or AAV13, or AAVrh10 capsid protein, or any chimera thereof
22. The rAAV of paragraph 20 or 21, wherein the capsid protein is an AAVrh10 capsid protein.
23. The rAAV of any one of paragraphs 20 to 22, wherein the rAAV is a self-complementary AAV (scAAV).
24. The rAAV of any one of paragraphs 20 to 22, wherein the rAAV is formulated for delivery to the central nervous system (CNS).
25. An isolated nucleic acid encoding the sequence set forth in any one of SEQ ID NO: 1-22.
26. A composition comprising an isolated nucleic acid of any of paragraphs 1-16.
27. A composition comprising a vector of paragraphs 17 or 18.
28. A composition comprising a rAAV cell of any of paragraphs 20-24.
29. A method for treating Huntington's disease in a subject in need thereof, the method comprising administering to a subject having or at risk of developing Huntington's disease a therapeutically effective amount of the isolated nucleic acid of any one of paragraphs 1 to 16, the rAAV of any one of paragraphs 20 to 24, or a composition of any of paragraphs 25-28.
30. The method of paragraph 29, wherein the subject comprises a huntingtin gene having more than 36 CAG repeats, more than 40 repeats, or more than 100 repeats.
31. The method of paragraph 29 or 30, wherein the subject is less than 20 years of age.
32. The method of any one of paragraphs 29 to 31, wherein the administration results in delivery of the isolated nucleic acid or rAAV to the central nervous system (CNS) of the subject.
33. The method of any one of paragraphs 29 to 32, wherein the administration is via injection, optionally intravenous injection or intrastriatal injection.
34. The method of any one of paragraphs 29 to 33, wherein the administration is via catheter or related device.
35. The method of paragraph 34, further comprising the step, prior to administering, of diagnosing a subject as having or at risk of developing Huntington's disease.
36. The method of paragraph 34, further comprising the step, prior to administering, of receiving the results of an assay that diagnoses a subject as having or at risk of developing Huntington's disease.

### EXAMPLES

### Example 1

In one aspect described herein are inhibitory RNAs that can be used for the treatment of Huntington's disease. In some embodiments of any of the aspects, the nucleic acid sequence of the inhibitory RNA comprises one of SEQ ID NO: 1 or SEQ ID NOs: 4-9 or a sequence that is at least 95% (e.g., at least 95%, at least 96%, at least 97%, at least 98%, or at least 99%) identical to the sequence of at least one of SEQ ID NO: 1 or SEQ ID NOs: 4-9 that maintains the same functions as SEQ ID NO: 1 or SEQ ID NOs: 4-9 (e.g., HTT inhibition).

Described here are constructs comprising artificial miRNAs (see e.g., FIG. 1). pEMBL-D(+)-Syn1-hCG intron is a control vector, which is inserted with empty human chorionic gonadotropin (hCG) intron (hCGin; see e.g., SEQ ID NO: 34) and driven with synapsin promoter (see e.g., SEQ ID NO: 31-32). Two copies of control miRNA precursor (random sequences or non-functional mutation) are inserted into hCGin in the vector pEMBL-D(+)-Syn1-hCGin-2×control pre-miR. Two copies of artificial pre-miR (see e.g., SEQ ID NO: 35; perfect match with 3′-UTR targeting sequences, including about 100-150 bp flanked upstream and downstream sequences) are cloned into between the hCG introns. The two copies of artificial miRNA sequences were inserted into human chorionic gonadotropin (hCG) introns, which can cut the inserters to form the precursors of miRNAs. The pre-miRNA is a precursor, which has a hairpin loop construct. The pre-miRNA is translated into cytoplasm with the help of exportin 5 (Exp5) and Ran-GTP. These miRNA precursors are further processed into mature miRNAs with the help of ribonuclease III enzyme Drosha in the nucleus and Dicer in the cytoplasm (e.g., Dicer cleaves the precursor into mature miRNA, which can be about 20-22 bp). The vector pEMBL-D(+)-Syn1-CYP46A1-hCGin-2×artificial pre-miR is a combo construct, which can produce both CYP46A1 and artificial miRNA at the same time. In order to identify whether the pre-miRNA could be processed into mature miRNA and combined with HTT targeting sequences including CAG expansions, which are perfectly complementary with mature miRNA, are inserted behind luciferase gene. For the limit of package size, small poly A is used in the constructs.

The sequences of the following are known in the art: pEMBL; synapsin promoter (Syn1); ITRs (e.g., from AAV1, AAV2, AAV3b, AAV4, AAV5, AAV6, AAV7, AAV8, AAV9, AAV10, AAV11, AAV12, AAV13, or AAVrh10); hCG intron; small polyA; CYP46A1; luciferase; and/or HTT targeting sequences (e.g., HTT-3′UTR/mutant).

Synapsin-1 (Syn1) is a member of the synapsin gene family. Synapsins encode neuronal phosphoproteins which associate with the cytoplasmic surface of synaptic vesicles. Family members are characterized by common protein domains, and they are implicated in synaptogenesis and the modulation of neurotransmitter release, suggesting a potential role in several neuropsychiatric diseases. Syn1 plays a role in regulation of axonogenesis and synaptogenesis. Syn1 protein serves as a substrate for several different protein kinases and phosphorylation may function in the regulation of this protein in the nerve terminal. Mutations in this gene may be associated with X-linked disorders with primary neuronal degeneration such as Rett syndrome. Alternatively spliced transcript variants encoding different isoforms have been identified. In some embodiments of any of the aspects, the Syn1 promoter can comprise a human promoter Syn1 (see e.g., the Syn1 promoter associated with NCBI ref numbers NG_008437.1 RefSeqGene Range 5001-52957; NM_006950.3; NP_008881.2; NM_133499.2; NP_598006.1; see e.g., SEQ ID NO: 31-32).

CYP46A1 is a member of the cytochrome P450 superfamily of enzymes. The cytochrome P450 proteins are monooxygenases which catalyze many reactions involved in drug metabolism and synthesis of cholesterol, steroids and other lipids. This endoplasmic reticulum protein is expressed in the brain, where it converts cholesterol to 24S-hydroxycholesterol. While cholesterol cannot pass the blood-brain barrier, 24S-hydroxycholesterol can be secreted in the brain into the circulation to be returned to the liver for catabolism. In some embodiments of any of the aspects, CYP46A1 can comprise a human CYP46A1 (see e.g., NCBI ref numbers NG_007963.1 RefSeqGene Range 4881-47884; NM_006668.2; NP_006659.1; see e.g., SEQ ID NOs: 26-27). CYP46A1, the rate-limiting enzyme for cholesterol degradation, is neuroprotective in Huntington's disease (see e.g., Boussicault et al., CYP46A1, the rate-limiting enzyme for cholesterol degradation, is neuroprotective in Huntington's disease, Brain. 2016 March, 139(Pt 3):953-70; Kacher et al., CYP46A1 gene therapy deciphers the role of brain cholesterol metabolism in Huntington's disease, Brain. 2019 Aug. 1; 142(8):2432-2450; the contents of each of which are incorporated herein by reference in their entireties).

Non-limiting examples of miRNAs of the present disclosure include SEQ ID NO: 1 or SEQ ID NOs: 4-9.

SEQ ID NO: 6
CGAGGCCGGGGCGGGGCACA
SEQ ID NO: 7
CGGGGCGGGGCCGTGGAGGG
SEQ ID NO: 8
ACTGTGCCACTATGTTTTCA
SEQ ID NO: 9
GCCTTCATCAGCTTTTCCAG
SEQ ID NO: 1
GCTGCTGCTGCTGCTGCTGC
SEQ ID NO: 4
TGCTGGAAGGACTTGAGGGA
SEQ ID NO: 5
TGTTGCTGCTGCTGCTGCTG

In some embodiments of any of the aspects, an miRNA comprises a sequence complementary to at least two (e.g., 2, 3, 4, 5, 6, 7, 8, 9, 10, 11, 12, 13, 14, 15, 16, 17, 18, 19, 20, 21, 22, 23, 24, or 25) continuous bases of the sequence set forth in SEQ ID NO: 25 flanked by a miRNA backbone sequence. In some embodiments of any of the aspects, an miRNA comprises a sequence complementary to at least two (e.g., 2, 3, 4, 5, 6, 7, 8, 9, 10, 11, 12, 13, 14, 15, 16, 17, 18, 19, 20, 21, 22, 23, 24, or 25) continuous bases of the sequence of an untranslated region (e.g., 5′ UTR, 3′UTR), exon, CAG repeat, or CAG jumper (e.g., CAG 5′ jumper, CAG 3′ jumper) associated with HTT (see e.g., NCBI Gene ID: 3064; e.g., SEQ ID NO: 25; see e.g. Tables 1 or 2) flanked by a miRNA backbone sequence.

TABLE 2
Location of target sequences

| | | | Species of |
|---|---|---|---|
| | Location of | | sequences |
| | synthesized | | targeted by |
| | artificial | | artificial |
| Regions | miRNAs | miRNA sequence | miRNAs |
| II | 5′-UTR | CGAGGCCGGGGCGGGGCACA (SEQ ID NO: 6) | Human |
| II | 5′-UTR | CGGGGCGGGGCCGTGGAGGG (SEQ ID NO: 7) | Human |
| III | Exon 1 | ACTGTGCCACTATGTTTTCA (SEQ ID NO: 8) | Human |
| III | Exon 1 | GCCTTCATCAGCTTTTCCAG (SEQ ID NO: 9) | Human |
| I | CAG repeats | GCTGCTGCTGCTGCTGCTGC (SEQ ID NO: 1) | Human |
| I | CAG 5′jumper | TGCTGGAAGGACTTGAGGGA (SEQ ID NO: 4) | Human |
| I | CAG 3′-jumper | TGTTGCTGCTGCTGCTGCTG (SEQ ID NO: 5) | Human |

SEQ ID NO: 25 Hunting-tin mRNA (Homo sapiens); NCBI Ref. Seq NM 002111.8 (see e.g., NG_009378.1 RefSeqGene, range 5001-174286 for an exemplary HTT gene)

GCTGCCGGGACGGGTCCAAGATGGACGGCCGCTCAGGTTCTGCTTTTACCTGCGGCCCAG
AGCCCCATTCATTGCCCCGGTGCTGAGCGGCGCCGCGAGTCGGCCCGAGGCCTCCGGGG
ACTGCCGTGCCGGGCGGGAGACCGCCATGGCGACCCTGGAAAAGCTGATGAAGGCCTTC
GAGTCCCTCAAGTCCTTCCAGCAGCAGCAGCAGCAGCAGCAGCAGCAGCAGCAGCAGCA
GCAGCAGCAGCAGCAGCAGCAGCAACAGCCGCCACCGCCGCCGCCGCCGCCGCCGCCTC
CTCAGCTTCCTCAGCCGCCGCCGCAGGCACAGCCGCTGCTGCCTCAGCCGCAGCCGCCCC
CGCCGCCGCCCCCGCCGCCACCCGGCCCGGCTGTGGCTGAGGAGCCGCTGCACCGACCA
AAGAAAGAACTTTCAGCTACCAAGAAAGACCGTGTGAATCATTGTCTGACAATATGTGA
AAACATAGTGGCACAGTCTGTCAGAAATTCTCCAGAATTTCAGAAACTTCTGGGCATCGC
TATGGAACTTTTTCTGCTGTGCAGTGATGACGCAGAGTCAGATGTCAGGATGGTGGCTGA
CGAATGCCTCAACAAAGTTATCAAAGCTTTGATGGATTCTAATCTTCCAAGGTTACAGCT
CGAGCTCTATAAGGAAATTAAAAAGAATGGTGCCCCTCGGAGTTTGCGTGCTGCCCTGTG
GAGGTTTGCTGAGCTGGCTCACCTGGTTCGGCCTCAGAAATGCAGGCCTTACCTGGTGAA
CCTTCTGCCGTGCCTGACTCGAACAAGCAAGAGACCCGAAGAATCAGTCCAGGAGACCT
TGGCTGCAGCTGTTCCCAAAATTATGGCTTCTTTTGGCAATTTTGCAAATGACAATGAAA
TTAAGGTTTTGTTAAAGGCCTTCATAGCGAACCTGAAGTCAAGCTCCCCCACCATTCGGC
GGACAGCGGCTGGATCAGCAGTGAGCATCTGCCAGCACTCAAGAAGGACACAATATTTC
TATAGTTGGCTACTAAATGTGCTCTTAGGCTTACTCGTTCCTGTCGAGGATGAACACTCC
ACTCTGCTGATTCTTGGCGTGCTGCTCACCCTGAGGTATTTGGTGCCCTTGCTGCAGCAGC
AGGTCAAGGACACAAGCCTGAAAGGCAGCTTCGGAGTGACAAGGAAAGAAATGGAAGT
CTCTCCTTCTGCAGAGCAGCTTGTCCAGGTTTATGAACTGACGTTACATCATACACAGCA
CCAAGACCACAATGTTGTGACCGGAGCCCTGGAGCTGTTGCAGCAGCTCTTCAGAACGC
CTCCACCCGAGCTTCTGCAAACCCTGACCGCAGTCGGGGGCATTGGGCAGCTCACCGCTG
CTAAGGAGGAGTCTGGTGGCCGAAGCCGTAGTGGGAGTATTGTGGAACTTATAGCTGGA
GGGGGTTCCTCATGCAGCCCTGTCCTTTCAAGAAAACAAAAAGGCAAAGTGCTCTTAGG
AGAAGAAGAAGCCTTGGAGGATGACTCTGAATCGAGATCGGATGTCAGCAGCTCTGCCT
TAACAGCCTCAGTGAAGGATGAGATCAGTGGAGAGCTGGCTGCTTCTTCAGGGGTTTCCA
CTCCAGGGTCAGCAGGTCATGACATCATCACAGAACAGCCACGGTCACAGCACACACTG
CAGGCGGACTCAGTGGATCTGGCCAGCTGTGACTTGACAAGCTCTGCCACTGATGGGGA
TGAGGAGGATATCTTGAGCCACAGCTCCAGCCAGGTCAGCGCCGTCCCATCTGACCCTGC
CATGGACCTGAATGATGGGACCCAGGCCTCGTCGCCCATCAGCGACAGCTCCCAGACCA
CCACCGAAGGGCCTGATTCAGCTGTTACCCCTTCAGACAGTTCTGAAATTGTGTTAGACG
GTACCGACAACCAGTATTTGGGCCTGCAGATTGGACAGCCCCAGGATGAAGATGAGGAA
GCCACAGGTATTCTTCCTGATGAAGCCTCGGAGGCCTTCAGGAACTCTTCCATGGCCCTT
CAACAGGCACATTTATTGAAAAACATGAGTCACTGCAGGCAGCCTTCTGACAGCAGTGTT
GATAAATTTGTGTTGAGAGATGAAGCTACTGAACCGGGTGATCAAGAAAACAAGCCTTG
CCGCATCAAAGGTGACATTGGACAGTCCACTGATGATGACTCTGCACCTCTTGTCCATTG
TGTCCGCCTTTTATCTGCTTCGTTTTTGCTAACAGGGGGAAAAAATGTGCTGGTTCCGGAC
AGGGATGTGAGGGTCAGCGTGAAGGCCCTGGCCCTCAGCTGTGTGGGAGCAGCTGTGGC
CCTCCACCCGGAATCTTTCTTCAGCAAACTCTATAAAGTTCCTCTTGACACCACGGAATA
CCCTGAGGAACAGTATGTCTCAGACATCTTGAACTACATCGATCATGGAGACCCACAGGT
TCGAGGAGCCACTGCCATTCTCTGTGGGACCCTCATCTGCTCCATCCTCAGCAGGTCCCG
CTTCCACGTGGGAGATTGGATGGGCACCATTAGAACCCTCACAGGAAATACATTTTCTTT
GGCGGATTGCATTCCTTTGCTGCGGAAAACACTGAAGGATGAGTCTTCTGTTACTTGCAA
GTTAGCTTGTACAGCTGTGAGGAACTGTGTCATGAGTCTCTGCAGCAGCAGCTACAGTGA
GTTAGGACTGCAGCTGATCATCGATGTGCTGACTCTGAGGAACAGTTCCTATTGGCTGGT
GAGGACAGAGCTTCTGGAAACCCTTGCAGAGATTGACTTCAGGCTGGTGAGCTTTTTGGA
GGCAAAAGCAGAAAACTTACACAGAGGGGCTCATCATTATACAGGGCTTTTAAAACTGC
AAGAACGAGTGCTCAATAATGTTGTCATCCATTTGCTTGGAGATGAAGACCCCAGGGTGC
GACATGTTGCCGCAGCATCACTAATTAGGCTTGTCCCAAAGCTGTTTTATAAATGTGACC
AAGGACAAGCTGATCCAGTAGTGGCCGTGGCAAGAGATCAAAGCAGTGTTTACCTGAAA
CTTCTCATGCATGAGACGCAGCCTCCATCTCATTTCTCCGTCAGCACAATAACCAGAATA
TATAGAGGCTATAACCTACTACCAAGCATAACAGACGTCACTATGGAAAATAACCTTTCA
AGAGTTATTGCAGCAGTTTCTCATGAACTAATCACATCAACCACCAGAGCACTCACATTT
GGATGCTGTGAAGCTTTGTGTCTTCTTTCCACTGCCTTCCCAGTTTGCATTTGGAGTTTAG
GTTGGCACTGTGGAGTGCCTCCACTGAGTGCCTCAGATGAGTCTAGGAAGAGCTGTACCG
TTGGGATGGCCACAATGATTCTGACCCTGCTCTCGTCAGCTTGGTTCCCATTGGATCTCTC
AGCCCATCAAGATGCTTTGATTTTGGCCGGAAACTTGCTTGCAGCCAGTGCTCCCAAATC
TCTGAGAAGTTCATGGGCCTCTGAAGAAGAAGCCAACCCAGCAGCCACCAAGCAAGAGG
AGGTCTGGCCAGCCCTGGGGGACCGGGCCCTGGTGCCCATGGTGGAGCAGCTCTTCTCTC
ACCTGCTGAAGGTGATTAACATTTGTGCCCACGTCCTGGATGACGTGGCTCCTGGACCCG
CAATAAAGGCAGCCTTGCCTTCTCTAACAAACCCCCCTTCTCTAAGTCCCATCCGACGAA
AGGGGAAGGAGAAAGAACCAGGAGAACAAGCATCTGTACCGTTGAGTCCCAAGAAAGG
CAGTGAGGCCAGTGCAGCTTCTAGACAATCTGATACCTCAGGTCCTGTTACAACAAGTAA
ATCCTCATCACTGGGGAGTTTCTATCATCTTCCTTCATACCTCAAACTGCATGATGTCCTG
AAAGCTACACACGCTAACTACAAGGTCACGCTGGATCTTCAGAACAGCACGGAAAAGTT
TGGAGGGTTTCTCCGCTCAGCCTTGGATGTTCTTTCTCAGATACTAGAGCTGGCCACACT
GCAGGACATTGGGAAGTGTGTTGAAGAGATCCTAGGATACCTGAAATCCTGCTTTAGTCG
AGAACCAATGATGGCAACTGTTTGTGTTCAACAATTGTTGAAGACTCTCTTTGGCACAAA
CTTGGCCTCCCAGTTTGATGGCTTATCTTCCAACCCCAGCAAGTCACAAGGCCGAGCACA
GCGCCTTGGCTCCTCCAGTGTGAGGCCAGGCTTGTACCACTACTGCTTCATGGCCCCGTA
CACCCACTTCACCCAGGCCCTCGCTGACGCCAGCCTGAGGAACATGGTGCAGGCGGAGC
AGGAGAACGACACCTCGGGATGGTTTGATGTCCTCCAGAAAGTGTCTACCCAGTTGAAG
ACAAACCTCACGAGTGTCACAAAGAACCGTGCAGATAAGAATGCTATTCATAATCACAT
TCGTTTGTTTGAACCTCTTGTTATAAAAGCTTTAAAACAGTACACGACTACAACATGTGT
GCAGTTACAGAAGCAGGTTTTAGATTTGCTGGCGCAGCTGGTTCAGTTACGGGTTAATTA
CTGTCTTCTGGATTCAGATCAGGTGTTTATTGGCTTTGTATTGAAACAGTTTGAATACATT
GAAGTGGGCCAGTTCAGGGAATCAGAGGCAATCATTCCAAACATCTTTTTCTTCTTGGTA
TTACTATCTTATGAACGCTATCATTCAAAACAGATCATTGGAATTCCTAAAATCATTCAG
CTCTGTGATGGCATCATGGCCAGTGGAAGGAAGGCTGTGACACATGCCATACCGGCTCT
GCAGCCCATAGTCCACGACCTCTTTGTATTAAGAGGAACAAATAAAGCTGATGCAGGAA
AAGAGCTTGAAACCCAAAAAGAGGTGGTGGTGTCAATGTTACTGAGACTCATCCAGTAC
CATCAGGTGTTGGAGATGTTCATTCTTGTCCTGCAGCAGTGCCACAAGGAGAATGAAGAC
AAGTGGAAGCGACTGTCTCGACAGATAGCTGACATCATCCTCCCAATGTTAGCCAAACA
GCAGATGCACATTGACTCTCATGAAGCCCTTGGAGTGTTAAATACATTATTTGAGATTTT
GGCCCCTTCCTCCCTCCGTCCGGTAGACATGCTTTTACGGAGTATGTTCGTCACTCCAAAC
ACAATGGCGTCCGTGAGCACTGTTCAACTGTGGATATCGGGAATTCTGGCCATTTTGAGG
GTTCTGATTTCCCAGTCAACTGAAGATATTGTTCTTTCTCGTATTCAGGAGCTCTCCTTCT
CTCCGTATTTAATCTCCTGTACAGTAATTAATAGGTTAAGAGATGGGGACAGTACTTCAA
CGCTAGAAGAACACAGTGAAGGGAAACAAATAAAGAATTTGCCAGAAGAAACATTTTCA
AGGTTTCTATTACAACTGGTTGGTATTCTTTTAGAAGACATTGTTACAAAACAGCTGAAG
GTGGAAATGAGTGAGCAGCAACATACTTTCTATTGCCAGGAACTAGGCACACTGCTAAT
GTGTCTGATCCACATCTTCAAGTCTGGAATGTTCCGGAGAATCACAGCAGCTGCCACTAG
GCTGTTCCGCAGTGATGGCTGTGGCGGCAGTTTCTACACCCTGGACAGCTTGAACTTGCG
GGCTCGTTCCATGATCACCACCCACCCGGCCCTGGTGCTGCTCTGGTGTCAGATACTGCT
GCTTGTCAACCACACCGACTACCGCTGGTGGGCAGAAGTGCAGCAGACCCCGAAAAGAC
ACAGTCTGTCCAGCACAAAGTTACTTAGTCCCCAGATGTCTGGAGAAGAGGAGGATTCT
GACTTGGCAGCCAAACTTGGAATGTGCAATAGAGAAATAGTACGAAGAGGGGCTCTCAT
TCTCTTCTGTGATTATGTCTGTCAGAACCTCCATGACTCCGAGCACTTAACGTGGCTCATT
GTAAATCACATTCAAGATCTGATCAGCCTTTCCCACGAGCCTCCAGTACAGGACTTCATC
AGTGCCGTTCATCGGAACTCTGCTGCCAGCGGCCTGTTCATCCAGGCAATTCAGTCTCGT
TGTGAAAACCTTTCAACTCCAACCATGCTGAAGAAAACTCTTCAGTGCTTGGAGGGGATC
CATCTCAGCCAGTCGGGAGCTGTGCTCACGCTGTATGTGGACAGGCTTCTGTGCACCCCT
TTCCGTGTGCTGGCTCGCATGGTCGACATCCTTGCTTGTCGCCGGGTAGAAATGCTTCTG
GCTGCAAATTTACAGAGCAGCATGGCCCAGTTGCCAATGGAAGAACTCAACAGAATCCA
GGAATACCTTCAGAGCAGCGGGCTCGCTCAGAGACACCAAAGGCTCTATTCCCTGCTGG
ACAGGTTTCGTCTCTCCACCATGCAAGACTCACTTAGTCCCTCTCCTCCAGTCTCTTCCCA
CCCGCTGGACGGGGATGGGCACGTGTCACTGGAAACAGTGAGTCCGGACAAAGACTGGT
ACGTTCATCTTGTCAAATCCCAGTGTTGGACCAGGTCAGATTCTGCACTGCTGGAAGGTG
CAGAGCTGGTGAATCGGATTCCTGCTGAAGATATGAATGCCTTCATGATGAACTCGGAGT
TCAACCTAAGCCTGCTAGCTCCATGCTTAAGCCTAGGGATGAGTGAAATTTCTGGTGGCC
AGAAGAGTGCCCTTTTTGAAGCAGCCCGTGAGGTGACTCTGGCCCGTGTGAGCGGCACC
GTGCAGCAGCTCCCTGCTGTCCATCATGTCTTCCAGCCCGAGCTGCCTGCAGAGCCGGCG
GCCTACTGGAGCAAGTTGAATGATCTGTTTGGGGATGCTGCACTGTATCAGTCCCTGCCC
ACTCTGGCCCGGGCCCTGGCACAGTACCTGGTGGTGGTCTCCAAACTGCCCAGTCATTTG
CACCTTCCTCCTGAGAAAGAGAAGGACATTGTGAAATTCGTGGTGGCAACCCTTGAGGC
CCTGTCCTGGCATTTGATCCATGAGCAGATCCCGCTGAGTCTGGATCTCCAGGCAGGGCT
GGACTGCTGCTGCCTGGCCCTGCAGCTGCCTGGCCTCTGGAGCGTGGTCTCCTCCACAGA
GTTTGTGACCCACGCCTGCTCCCTCATCTACTGTGTGCACTTCATCCTGGAGGCCGTTGCA
GTGCAGCCTGGAGAGCAGCTTCTTAGTCCAGAAAGAAGGACAAATACCCCAAAAGCCAT
CAGCGAGGAGGAGGAGGAAGTAGATCCAAACACACAGAATCCTAAGTATATCACTGCA
GCCTGTGAGATGGTGGCAGAAATGGTGGAGTCTCTGCAGTCGGTGTTGGCCTTGGGTCAT
AAAAGGAATAGCGGCGTGCCGGCGTTTCTCACGCCATTGCTAAGGAACATCATCATCAG
CCTGGCCCGCCTGCCCCTTGTCAACAGCTACACACGTGTGCCCCCACTGGTGTGGAAGCT
TGGATGGTCACCCAAACCGGGAGGGGATTTTGGCACAGCATTCCCTGAGATCCCCGTGG
AGTTCCTCCAGGAAAAGGAAGTCTTTAAGGAGTTCATCTACCGCATCAACACACTAGGCT
GGACCAGTCGTACTCAGTTTGAAGAAACTTGGGCCACCCTCCTTGGTGTCCTGGTGACGC
AGCCCCTCGTGATGGAGCAGGAGGAGAGCCCACCAGAAGAAGACACAGAGAGGACCCA
GATCAACGTCCTGGCCGTGCAGGCCATCACCTCACTGGTGCTCAGTGCAATGACTGTGCC
TGTGGCCGGCAACCCAGCTGTAAGCTGCTTGGAGCAGCAGCCCCGGAACAAGCCTCTGA
AAGCTCTCGACACCAGGTTTGGGAGGAAGCTGAGCATTATCAGAGGGATTGTGGAGCAA
GAGATTCAAGCAATGGTTTCAAAGAGAGAGAATATTGCCACCCATCATTTATATCAGGC
ATGGGATCCTGTCCCTTCTCTGTCTCCGGCTACTACAGGTGCCCTCATCAGCCACGAGAA
GCTGCTGCTACAGATCAACCCCGAGCGGGAGCTGGGGAGCATGAGCTACAAACTCGGCC
AGGTGTCCATACACTCCGTGTGGCTGGGGAACAGCATCACACCCCTGAGGGAGGAGGAA
TGGGACGAGGAAGAGGAGGAGGAGGCCGACGCCCCTGCACCTTCGTCACCACCCACGTC
TCCAGTCAACTCCAGGAAACACCGGGCTGGAGTTGACATCCACTCCTGTTCGCAGTTTTT
GCTTGAGTTGTACAGCCGCTGGATCCTGCCGTCCAGCTCAGCCAGGAGGACCCCGGCCAT
CCTGATCAGTGAGGTGGTCAGATCCCTTCTAGTGGTCTCAGACTTGTTCACCGAGCGCAA
CCAGTTTGAGCTGATGTATGTGACGCTGACAGAACTGCGAAGGGTGCACCCTTCAGAAG
ACGAGATCCTCGCTCAGTACCTGGTGCCTGCCACCTGCAAGGCAGCTGCCGTCCTTGGGA
TGGACAAGGCCGTGGCGGAGCCTGTCAGCCGCCTGCTGGAGAGCACGCTCAGGAGCAGC
CACCTGCCCAGCAGGGTTGGAGCCCTGCACGGCGTCCTCTATGTGCTGGAGTGCGACCTG
CTGGACGACACTGCCAAGCAGCTCATCCCGGTCATCAGCGACTATCTCCTCTCCAACCTG
AAAGGGATCGCCCACTGCGTGAACATTCACAGCCAGCAGCACGTACTGGTCATGTGTGC
CACTGCGTTTTACCTCATTGAGAACTATCCTCTGGACGTAGGGCCGGAATTTTCAGCATC
AATAATACAGATGTGTGGGGTGATGCTGTCTGGAAGTGAGGAGTCCACCCCCTCCATCAT
TTACCACTGTGCCCTCAGAGGCCTGGAGCGCCTCCTGCTCTCTGAGCAGCTCTCCCGCCT
GGATGCAGAATCGCTGGTCAAGCTGAGTGTGGACAGAGTGAACGTGCACAGCCCGCACC
GGGCCATGGCGGCTCTGGGCCTGATGCTCACCTGCATGTACACAGGAAAGGAGAAAGTC
AGTCCGGGTAGAACTTCAGACCCTAATCCTGCAGCCCCCGACAGCGAGTCAGTGATTGTT
GCTATGGAGCGGGTATCTGTTCTTTTTGATAGGATCAGGAAAGGCTTTCCTTGTGAAGCC
AGAGTGGTGGCCAGGATCCTGCCCCAGTTTCTAGACGACTTCTTCCCACCCCAGGACATC
ATGAACAAAGTCATCGGAGAGTTTCTGTCCAACCAGCAGCCATACCCCCAGTTCATGGCC
ACCGTGGTGTATAAGGTGTTTCAGACTCTGCACAGCACCGGGCAGTCGTCCATGGTCCGG
GACTGGGTCATGCTGTCCCTCTCCAACTTCACGCAGAGGGCCCCGGTCGCCATGGCCACG
TGGAGCCTCTCCTGCTTCTTTGTCAGCGCGTCCACCAGCCCGTGGGTCGCGGCGATCCTC
CCACATGTCATCAGCAGGATGGGCAAGCTGGAGCAGGTGGACGTGAACCTTTTCTGCCT
GGTCGCCACAGACTTCTACAGACACCAGATAGAGGAGGAGCTCGACCGCAGGGCCTTCC
AGTCTGTGCTTGAGGTGGTTGCAGCCCCAGGAAGCCCATATCACCGGCTGCTGACTTGTT
TACGAAATGTCCACAAGGTCACCACCTGCTGAGCGCCATGGTGGGAGAGACTGTGAGGC
GGCAGCTGGGGCCGGAGCCTTTGGAAGTCTGCGCCCTTGTGCCCTGCCTCCACCGAGCCA
GCTTGGTCCCTATGGGCTTCCGCACATGCCGCGGGCGGCCAGGCAACGTGCGTGTCTCTG
CCATGTGGCAGAAGTGCTCTTTGTGGCAGTGGCCAGGCAGGGAGTGTCTGCAGTCCTGGT
GGGGCTGAGCCTGAGGCCTTCCAGAAAGCAGGAGCAGCTGTGCTGCACCCCATGTGGGT
GACCAGGTCCTTTCTCCTGATAGTCACCTGCTGGTTGTTGCCAGGTTGCAGCTGCTCTTGC
ATCTGGGCCAGAAGTCCTCCCTCCTGCAGGCTGGCTGTTGGCCCCTCTGCTGTCCTGCAG
TAGAAGGTGCCGTGAGCAGGCTTTGGGAACACTGGCCTGGGTCTCCCTGGTGGGGTGTG
CATGCCACGCCCCGTGTCTGGATGCACAGATGCCATGGCCTGTGCTGGGCCAGTGGCTGG
GGGTGCTAGACACCCGGCACCATTCTCCCTTCTCTCTTTTCTTCTCAGGATTTAAAATTTA
ATTATATCAGTAAAGAGATTAATTTTAACGTAACTCTTTCTATGCCCGTGTAAAGTATGT
GAATCGCAAGGCCTGTGCTGCATGCGACAGCGTCCGGGGTGGTGGACAGGGCCCCCGGC
CACGCTCCCTCTCCTGTAGCCACTGGCATAGCCCTCCTGAGCACCCGCTGACATTTCCGTT
GTACATGTTCCTGTTTATGCATTCACAAGGTGACTGGGATGTAGAGAGGCGTTAGTGGGC
AGGTGGCCACAGCAGGACTGAGGACAGGCCCCCATTATCCTAGGGGTGCGCTCACCTGC
AGCCCCTCCTCCTCGGGCACAGACGACTGTCGTTCTCCACCCACCAGTCAGGGACAGCAG
CCTCCCTGTCACTCAGCTGAGAAGGCCAGCCCTCCCTGGCTGTGAGCAGCCTCCACTGTG
TCCAGAGACATGGGCCTCCCACTCCTGTTCCTTGCTAGCCCTGGGGTGGCGTCTGCCTAG
GAGCTGGCTGGCAGGTGTTGGGACCTGCTGCTCCATGGATGCATGCCCTAAGAGTGTCAC
TGAGCTGTGTTTTGTCTGAGCCTCTCTCGGTCAACAGCAAAGCTTGGTGTCTTGGCACTGT
TAGTGACAGAGCCCAGCATCCCTTCTGCCCCCGTTCCAGCTGACATCTTGCACGGTGACC
CCTTTTAGTCAGGAGAGTGCAGATCTGTGCTCATCGGAGACTGCCCCACGGCCCTGTCAG
AGCCGCCACTCCTATCCCCAGGCCAGGTCCCTGGACCAGCCTCCTGTTTGCAGGCCCAGA
GGAGCCAAGTCATTAAAATGGAAGTGGATTCTGGATGGCCGGGCTGCTGCTGATGTAGG
AGCTGGATTTGGGAGCTCTGCTTGCCGACTGGCTGTGAGACGAGGCAGGGGCTCTGCTTC
CTCAGCCCTAGAGGCGAGCCAGGCAAGGTTGGCGACTGTCATGTGGCTTGGTTTGGTCAT
GCCCGTCGATGTTTTGGGTATTGAATGTGGTAAGTGGAGGAAATGTTGGAACTCTGTGCA
GGTGCTGCCTTGAGACCCCCAAGCTTCCACCTGTCCCTCTCCTATGTGGCAGCTGGGGAG
CAGCTGAGATGTGGACTTGTATGCTGCCCACATACGTGAGGGGGAGCTGAAAGGGAGCC
CCTCCTCTGAGCAGCCTCTGCCAGGCCTGTATGAGGCTTTTCCCACCAGCTCCCAACAGA
GGCCTCCCCCAGCCAGGACCACCTCGTCCTCGTGGCGGGGCAGCAGGAGCGGTAGAAAG
GGGTCCGATGTTTGAGGAGGCCCTTAAGGGAAGCTACTGAATTATAACACGTAAGAAAA
TCACCATTCCGTATTGGTTGGGGGCTCCTGTTTCTCATCCTAGCTTTTTCCTGGAAAGCCC
GCTAGAAGGTTTGGGAACGAGGGGAAAGTTCTCAGAACTGTTGGCTGCTCCCCACCCGC
CTCCCGCCTCCCCCGCAGGTTATGTCAGCAGCTCTGAGACAGCAGTATCACAGGCCAGAT
GTTGTTCCTGGCTAGATGTTTACATTTGTAAGAAATAACACTGTGAATGTAAAACAGAGC
CATTCCCTTGGAATGCATATCGCTGGGCTCAACATAGAGTTTGTCTTCCTCTTGTTTACGA
CGTGATCTAAACCAGTCCTTAGCAAGGGGCTCAGAACACCCCGCTCTGGCAGTAGGTGTC
CCCCACCCCCAAAGACCTGCCTGTGTGCTCCGGAGATGAATATGAGCTCATTAGTAAAAA
TGACTTCACCCACGCATATACATAAAGTATCCATGCATGTGCATATAGACACATCTATAA
TTTTACACACACACCTCTCAAGACGGAGATGCATGGCCTCTAAGAGTGCCCGTGTCGGTT
CTTCCTGGAAGTTGACTTTCCTTAGACCCGCCAGGTCAAGTTAGCCGCGTGACGGACATC
CAGGCGTGGGACGTGGTCAGGGCAGGGCTCATTCATTGCCCACTAGGATCCCACTGGCG
AAGATGGTCTCCATATCAGCTCTCTGCAGAAGGGAGGAAGACTTTATCATGTTCCTAAAA
ATCTGTGGCAAGCACCCATCGTATTATCCAAATTTTGTTGCAAATGTGATTAATTTGGTTG
TCAAGTTTTGGGGGTGGGCTGTGGGGAGATTGCTTTTGTTTTCCTGCTGGTAATATCGGG
AAAGATTTTAATGAAACCAGGGTAGAATTGTTTGGCAATGCACTGAAGCGTGTTTCTTTC
CCAAAATGTGCCTCCCTTCCGCTGCGGGCCCAGCTGAGTCTATGTAGGTGATGTTTCCAG
CTGCCAAGTGCTCTTTGTTACTGTCCACCCTCATTTCTGCCAGCGCATGTGTCCTTTCAAG
GGGAAAATGTGAAGCTGAACCCCCTCCAGACACCCAGAATGTAGCATCTGAGAAGGCCC
TGTGCCCTAAAGGACACCCCTCGCCCCCATCTTCATGGAGGGGGTCATTTCAGAGCCCTC
GGAGCCAATGAACAGCTCCTCCTCTTGGAGCTGAGATGAGCCCCACGTGGAGCTCGGGA
CGGATAGTAGACAGCAATAACTCGGTGTGTGGCCGCCTGGCAGGTGGAACTTCCTCCCGT
TGCGGGGTGGAGTGAGGTTAGTTCTGTGTGTCTGGTGGGTGGAGTCAGGCTTCTCTTGCT
ACCTGTGAGCATCCTTCCCAGCAGACATCCTCATCGGGCTTTGTCCCTCCCCCGCTTCCTC
CCTCTGCGGGGAGGACCCGGGACCACAGCTGCTGGCCAGGGTAGACTTGGAGCTGTCCT
CCAGAGGGGTCACGTGTAGGAGTGAGAAGAAGGAAGATCTTGAGAGCTGCTGAGGGAC
CTTGGAGAGCTCAGGATGGCTCAGACGAGGACACTCGCTTGCCGGGCCTGGGCCTCCTG
GGAAGGAGGGAGCTGCTCAGAATGCCGCATGACAACTGAAGGCAACCTGGAAGGTTCA
GGGGCCGCTCTTCCCCCATGTGCCTGTCACGCTCTGGTGCAGTCAAAGGAACGCCTTCCC
CTCAGTTGTTTCTAAGAGCAGAGTCTCCCGCTGCAATCTGGGTGGTAACTGCCAGCCTTG
GAGGATCGTGGCCAACGTGGACCTGCCTACGGAGGGTGGGCTCTGACCCAAGTGGGGCC
TCCTTGTCCAGGTCTCACTGCTTTGCACCGTGGTCAGAGGGACTGTCAGCTGAGCTTGAG
CTCCCCTGGAGCCAGCAGGGCTGTGATGGGCGAGTCCCGGAGCCCCACCCAGACCTGAA
TGCTTCTGAGAGCAAAGGGAAGGACTGACGAGAGATGTATATTTAATTTTTTAACTGCTG
CAAACATTGTACATCCAAATTAAAGGAAAAAAATGGAAACCATCAAAAAAAAAAAAAA
AAAA

### Example 2

AAV-Mediated Artificial miRNAs and their Application on Huntington Disease (HD)

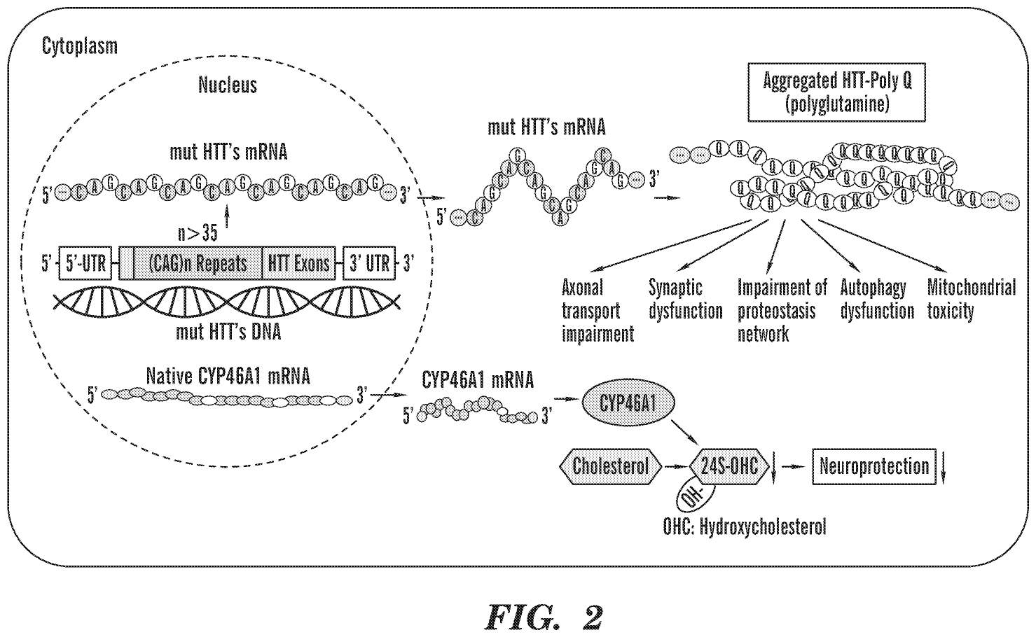

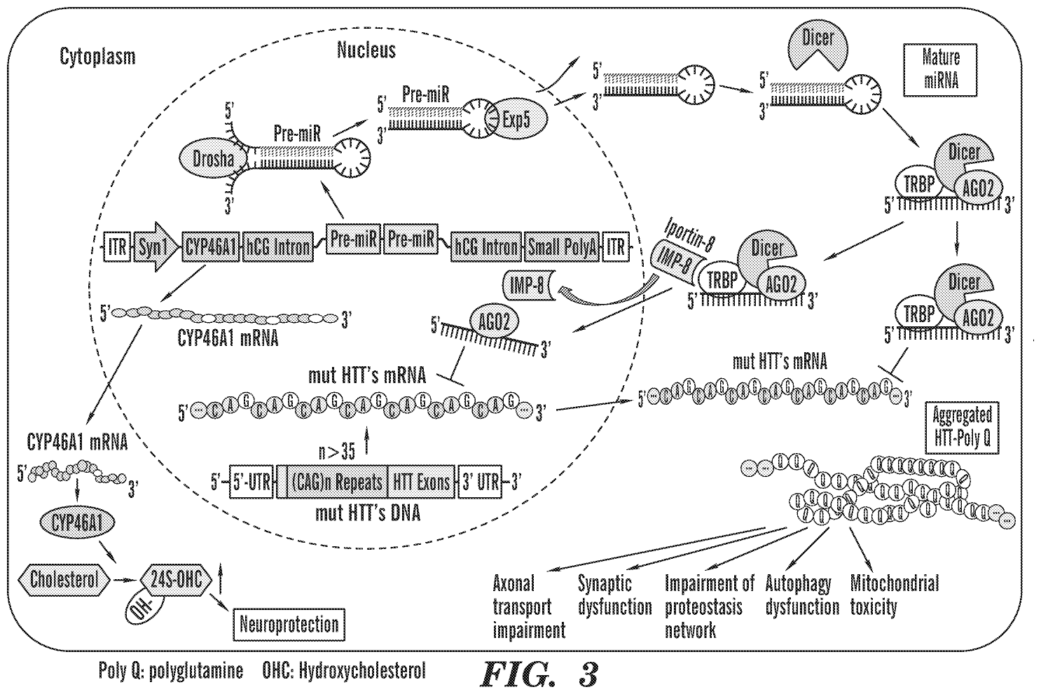

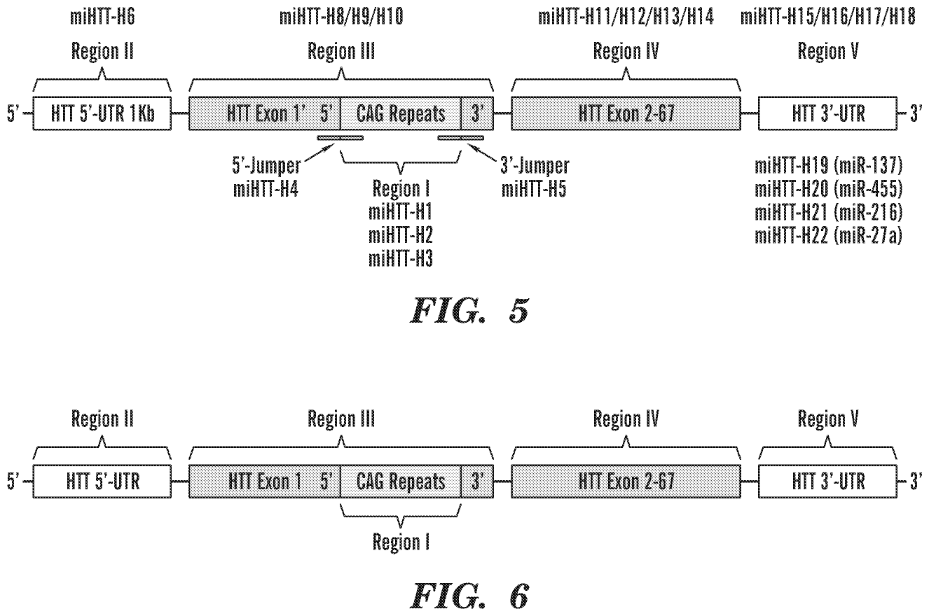

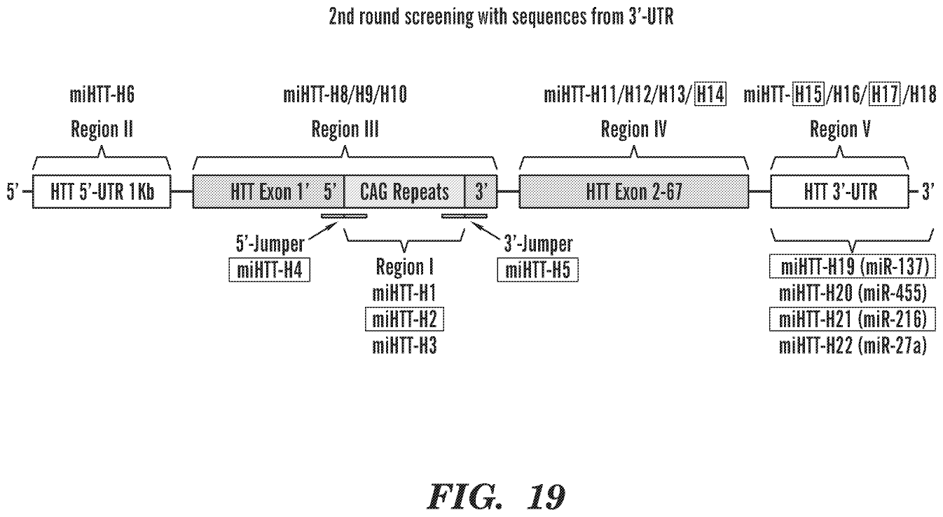

Described herein are artificial miRNAs that can be used to treat HD (see e.g., FIG. 2-3). Several screens were performed to identify and test the artificial miRNAs. Overall, the process of screening the artificial miRNAs for HD comprises: (1) designing and synthesizing the artificial miRs (e.g., 24 artificial miRNA constructs; see e.g., Table 1, SEQ ID NOs: 1-24, FIG. 1, FIG. 5-6, FIG. 8A, FIG. 19). The first in vitro screen comprises: (2) co-transfecting plasmids in vitro (e.g., screening 24 miRs via plasmid co-transfection in 293 cells; see e.g., FIG. 7, FIG. 8B, FIG. 16-18); (3) with the top ˜5 candidates from step (2) performing AAVRH10-mediated infection in vitro (e.g., using the CMV promoter; see e.g., FIG. 10-14, FIG. 20-21); and/or (4) with the top ˜2-3 candidates from step (3) performing AAVRH10-mediated infection driven by a neuron specific promoter (e.g., hSyn1promoter, with optional CYP46A1 co-expression) to test the efficiency of the miRs in vitro. (5) The second in vivo screen comprises AAVRH10-mediated treatment in vivo, comprising testing the artificial miRNAs mediated by AAVrh10 in transgenic (Tg) mice (e.g., Hu-128 or B6CBA-R6/2). (6) An evaluation is then performed of the efficiency and safety of the artificial miRs and their combination with CYP46A1. See e.g., FIG. 4, FIG. 9, FIG. 15.

Phase I: Screening for the Artificial miRNAs Located at Regions I-III

Artificial miRNAs located in Regions I-III include miHTT-H1 (SEQ ID NO: 1); miHTT-H2 (SEQ ID NO: 2); miHTT-H3 (SEQ ID NO: 3); miHTT-H4 (SEQ ID NO: 4); miHTT-H5 (SEQ ID NO: 5); miHTT-H6 (SEQ ID NO: 6); miHTT-H7 (SEQ ID NO: 7); miHTT-H8 (SEQ ID NO: 8); miHTT-H9 (SEQ ID NO: 9); or miHTT-H10 (SEQ ID NO: 10); see e.g., Table 1.

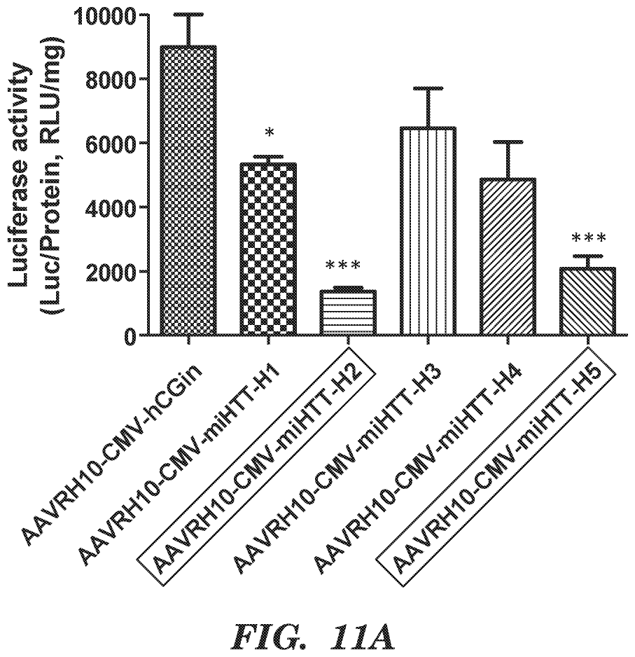

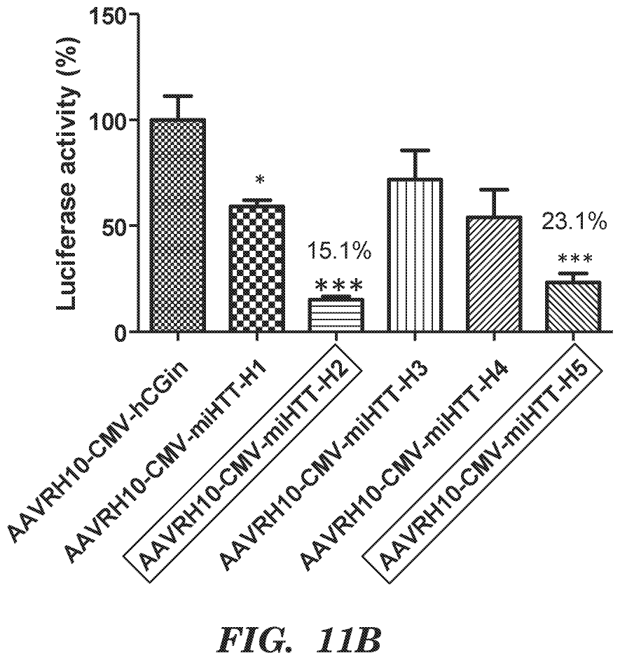

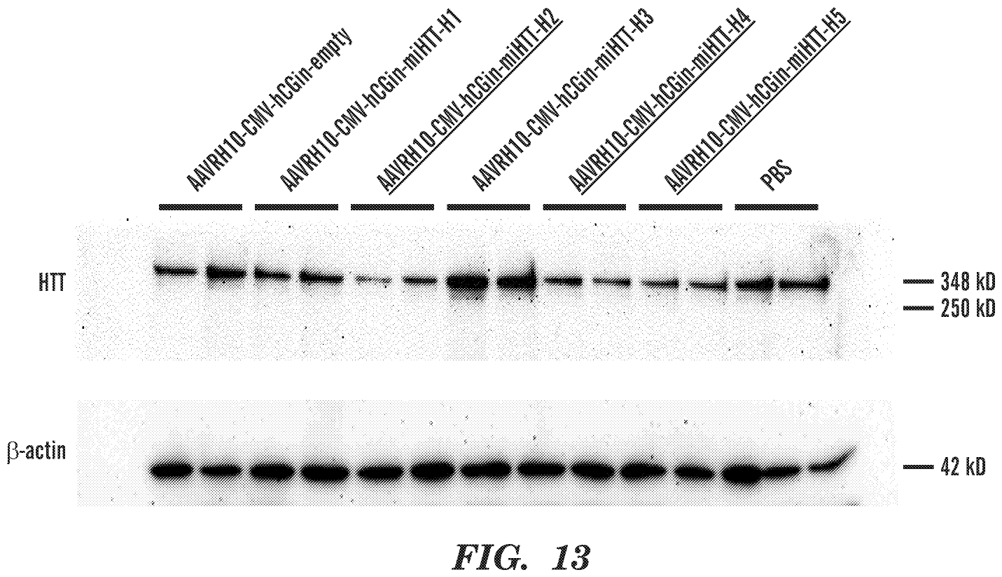

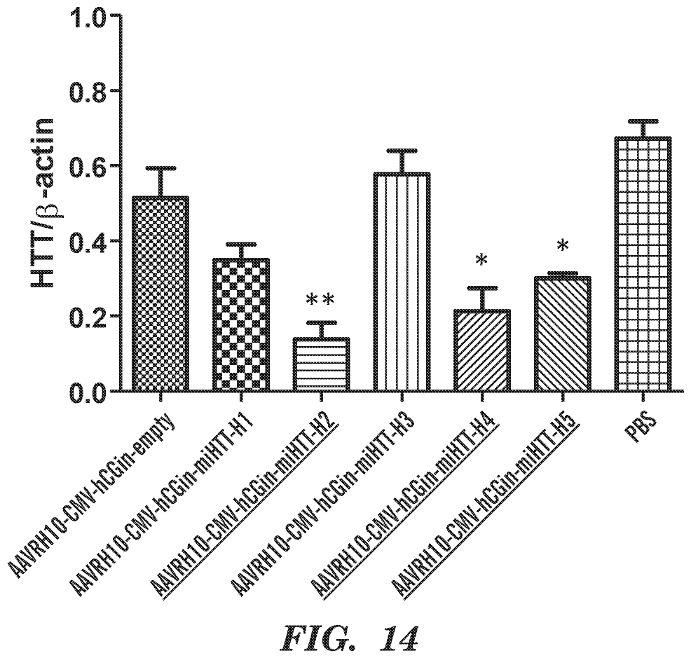

The process of screening the artificial miRNAs located in Regions I-III for HD comprises: (1) designing and synthesizing the artificial miRs (e.g., 9-10 artificial miRNA constructs; see e.g., Table 1, SEQ ID NOs: 1-10, FIG. 8A). The first in vitro screen comprises: (2) co-transfecting plasmids in vitro (e.g., screening 9-10 miRs via plasmid co-transfection in 293 cells; see e.g., FIG. 8B); (3) with the top ˜5 candidates from step (2) performing AAVRH10-mediated infection in vitro (e.g., using the CMV promoter; see e.g., FIG. 10-14); and/or (4) with the top 2 candidates from step (3) (e.g., miR-H2 and miR-H5) performing AAVRH10-mediated infection driven by a neuron specific promoter (e.g., hSyn1 promoter, with optional CYP46A1 co-expression) to test the efficiency of the miRs in vitro. (5) The second in vivo screen comprises AAVRH10-mediated treatment in vivo, comprising testing the artificial miRNAs mediated by AAVrh10 in Tg mice (e.g., Hu-128 or B6CBA-R612). (6) An evaluation is then performed of the efficiency and safety of the artificial miRs with or, without their combination with CYP46A1. See e.g., FIG. 9. The experiments described herein identified the following artificial miRNAs as especially effective: miHTT-H2 (SEQ ID NO: 2); miHTT-H4 (SEQ ID NO: 4); or miHTT-H5 (SEQ ID NO: 5); see e.g., FIG. 8A-8B, FIG. 11A-11B, FIG. 13-14. In particular, as depicted in FIG. 13 and FIG. 14, miHTT-H2, H4 and H5 efficiently downregulates HTT expression in a neural cell line U87 as compared to the HTT expression with empty vector (without the miRNA) or, other miRNAs tested e.g., miHTT-H1 or, miHTT-H3.

Phase II: Screening for the Artificial miRNAs Located at Regions IV-V

miRNAs located in Regions IV-V include miHTT-H11 (SEQ ID NO: 11); miHTT-H12 (SEQ ID NO: 12); miHTT-H13 (SEQ ID NO: 13); miHTT-H14 (SEQ ID NO: 14); miHTT-H15 (SEQ ID NO: 15); miHTT-H16 (SEQ ID NO: 16); miHTT-H17 (SEQ ID NO: 17); miHTT-H18 (SEQ ID NO: 18); miHTT-H19 (SEQ ID NO: 19; miR-137); miHTT-H20 (SEQ ID NO: 20; miR-455); miHTT-H21 (SEQ ID NO: 21; miR-216); or miHTT-H22 (SEQ ID NO: 22; miR-27a); see e.g., Table 1. The following miRs from Phase I can be used as positive controls: miHTT-H2 (SEQ ID NO: 2); miHTT-H4 (SEQ ID NO: 4); or miHTT-H5 (SEQ ID NO: 5).

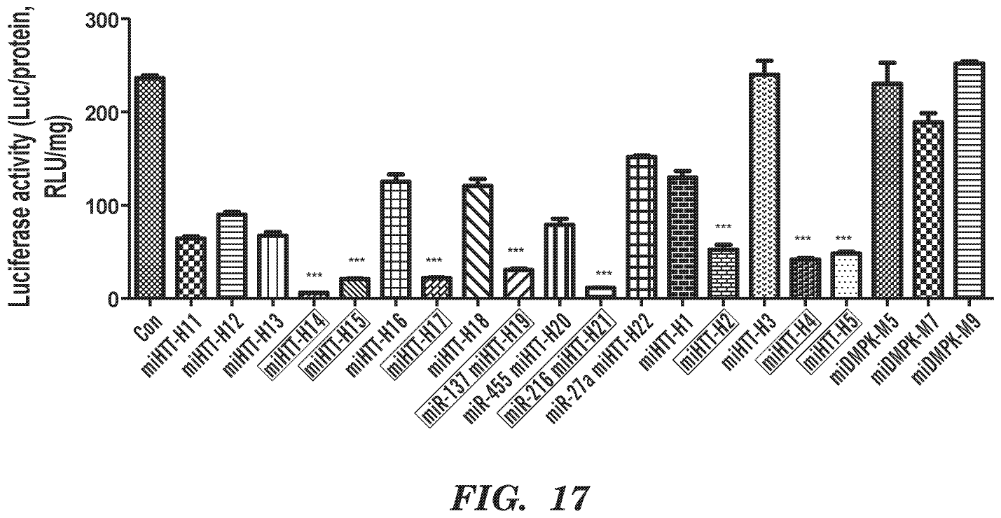

The process of screening the miRNAs located in Regions IV-V for HD comprises: (1) designing and synthesizing the artificial miRs (e.g., 12 artificial miRNA constructs; see e.g., Table 1, SEQ ID NOs: 11-22, FIG. 19). The first in vitro screen comprises: (2) co-transfecting plasmids in vitro (e.g., screening 12 miRs via plasmid co-transfection in 293 cells; see e.g., FIG. 16-18); (3) with the top ˜5 candidates from step (2) (e.g., miHTT-H14; miHTT-H15; miHTT-H17; miHTT-H19; and miHTT-H21) performing AAVRH10-mediated infection in vitro (e.g., using the CMV promoter; see e.g., FIG. 20-21); and/or (4) with the top ˜2-3 candidates from step (3) performing AAVRH10-mediated infection driven by a neuron specific promoter (e.g., hSyn1promoter, with optional CYP46A1 co-expression) to test the efficiency of the miRs in vitro. (5) The second in vivo screen comprises AAVRH10-mediated treatment in vivo, comprising testing the artificial miRNAs mediated by AAVrh10 in Tg mice (e.g., Hu-128). (6) An evaluation is then performed of the efficiency and safety of the artificial miRs with or, without their combination with CYP46A1. See e.g., FIG. 15. The experiments described herein identified the following artificial miRNAs as especially effective: miHTT-H14 (SEQ ID NO: 14); miHTT-H15 (SEQ ID NO: 15); and miHTT-H17 (SEQ ID NO: 17); along with the human-expressed miRNAs miHTT-H19 (SEQ ID NO: 19; miR-137) and miHTT-H21 (SEQ ID NO: 21; miR-216); see e.g., FIG. 17-19.

miR-137, miR-455, miR-216, and miR-27a (e.g., miHTT-H19-H22, SEQ ID NOs: 19-22) are examples of human-expressed miRNAs that were tested for efficacy in downregulating HTT. The inhibitory activity of these sequences against HTT were not previously known. As shown herein, miR-137 (miHTT-H19, SEQ ID NO: 19) and miR-216 (miHTT-H21, SEQ ID NO: 21) were two especially efficacious candidates to target the HTT 3′-UTR.

miR-137 (see e.g., miHTT-H19, SEQ ID NO: 19) is located on human chromosome 1p22 and has been implicated to act as a tumor suppressor in several cancer types including colorectal cancer, squamous cell carcinoma and melanoma via cell cycle control. miR-137 is shown to regulate neural stem cell proliferation and differentiation in mouse embryonic stem cells, and neuronal maturation, including regulation of dendrite length, branch points, end points, and spine density in mouse adult hippocampal neuroprogenitor-derived and mouse fetal hippocampus neurons. Diseases associated with miR455 include endometrial serous adenocarcinoma and Pettigrew Syndrome.

miR-455 (see e.g., miHTT-H20, SEQ ID NO: 20) is located on human chromosome 9q32. Diseases associated with miR-455 include endometrial serous adenocarcinoma and Pettigrew Syndrome. miR-216 (see e.g., miHTT-H21, SEQ ID NO: 21) is located on human chromosome 2p16.1. Diseases associated with miR-216 include microvascular complications of diabetes and pancreatic ductal adenocarcinoma. miR-27a (see e.g., miHTT-H22, SEQ ID NO: 22) is located on human chromosome 19p13.12. Diseases associated with miR27A include leukemia and gastric cancer. miR-27a is used herein as a positive control and has been reported to reduce mutant HTT aggregation in vitro; see e.g., Ban et al., Biochemical and Biophysical Research Communications 488(2), 2017, 316-321.

Phase III: Testing of Additional Artificial miRNAs Located at Region V Against miRNAs Identified in Phases II and II

Additional artificial miRNAs located in Region V include miR-451a (SEQ ID NO: 23) or miR-155 (SEQ ID NO: 24); see e.g., Table 1. miR-451a (SEQ ID NO: 23) is located on human chromosome 17q11.2. miR-451 regulates the drug-transporter protein P-glycoprotein, potentially promoting resistance to the chemotherapy drug Paclitaxel. Diseases associated with miR451A include glioma susceptibility and gastric cancer. miR-155 (see e.g., SEQ ID NO: 24) is located on human chromosome 21q21.3. Exogenous molecular control in vivo of miR-155 expression may inhibit malignant growth, viral infections, and enhance the progression of cardiovascular diseases. Diseases associated with miR155 include diffuse large B-cell lymphoma and pancreatic ductal adenocarcinoma. See e.g., U.S. Pat. No. 10,767,180 for discussion of these additional artificial miRNAs miR-451a and miR-155, the contents of which is incorporated herein by reference in its entirety. Without wishing to be bound by theory, it is expected that at least one or more miRNAs as disclosed herein, e.g. in Table 1, is better in inhibiting target gene, e.g. HTT, when compared with the inhibitory efficiency of miR-451a or miR-155 targeting the same target gene.

The following artificial miRs from Phase I and II can be tested against the additional miRNAs noted above: miHTT-H2 (SEQ ID NO: 2); miHTT-H4 (SEQ ID NO: 4); and miHTT-H5 (SEQ ID NO: 5); miHTT-H14 (SEQ ID NO: 14); miHTT-H15 (SEQ ID NO: 15); miHTT-H17 (SEQ ID NO: 17); miHTT-H19 (SEQ ID NO: 19; miR-137); or miHTT-H21 (SEQ ID NO: 21; miR-216).

The process of testing the additional artificial miRNAs for HD against the artificial mRNAs identified in Phases I and II comprises: (1) designing and synthesizing the artificial miRs (e.g., 2 artificial miRNA constructs; see e.g., Table 1, SEQ ID NOs: 23-24, FIG. 6). The first in vitro test comprises: (2) co-transfecting plasmids in vitro (e.g., screening 2 miRs via plasmid co-transfection in 293 cells); (3) performing AAVRH10-mediated infection in vitro (e.g., using the CMV promoter); and/or (4) performing AAVRH10-mediated infection driven by a neuron specific promoter (e.g., hSyn1promoter, with optional CYP46A1 co-expression) to test the efficiency of the miRs in vitro. (5) The second in vivo test comprises AAVRH10-mediated treatment in vivo, comprising testing the artificial miRNAs mediated by AAVrh10 in Tg mice (e.g., Hu-128 or B6CBA-R6/2). (6) An evaluation is then performed of the efficiency and safety of the artificial miRs with or without their combination with CYP46A1.

Furthermore, the efficacy of the additional miRNAs is compared to the efficacy of the artificial miRNAs identified in Phases I and II (e.g., miHTT-H2 (SEQ ID NO: 2); miHTT-H4 (SEQ ID NO: 4); and miHTT-H5 (SEQ ID NO: 5); miHTT-H14 (SEQ ID NO: 14); miHTT-H15 (SEQ ID NO: 15); miHTT-H17 (SEQ ID NO: 17); miHTT-H19 (SEQ ID NO: 19; miR-137); or miHTT-H21 (SEQ ID NO: 21; miR-216)). See e.g., FIG. 4. Without wishing to be bound by theory, it is anticipated that at least one of SEQ ID NOs: 2, 4, 5, 14, 15, 17, or 21 can exhibit increased efficiency and/or efficacy (e.g., at reducing HTT mRNA or protein levels or activity) in vitro or in vivo compared to SEQ ID NO: 23 or 24.

The Choice of Artificial miRNAs for Different HTT Mouse Models

TABLE 3
HTT mouse models (“✓” indicates that the region
is present in the mouse model; accordingly, miRNAs that target
each indicated region can be used in the indicated mouse model).

| | HTT Regions (see e.g., FIG. 6) | | | | |
|---|---|---|---|---|---|
| | Region | Region | Region | Region | Region |
| HTT models | I | II | III | IV | V |
| Hu128 | ✓ | ✓ | ✓ | ✓ | ✓ |
| B6CBA-R6/2 (CAG | ✓ | ✓ | ✓ | | |
| 120 +/− 5) or B6CBA- | | | | | |
| Tg(HDexon1)62Gpb/3J | | | | | |
| B6CBA-R6/2 (CAG | ✓ | ✓ | ✓ | | |
| 160 +/− 5) or B6CBA- | | | | | |
| Tg(HDexon1)62Gpb/1J | | | | | |

The transgene mouse model Hu128 has a knock in of the full-size human HTT gene, including the 5′ untranslated region (5′-UTR) and the 3′-UTR (see e.g., FIG. 6 and Table 3). Other transgene mouse models (e.g., B6CBA-R6/2 (CAG 120+/−5); B6CBA-Tg(HDexon1)62Gpb/3J; B6CBA-R6/2 (CAG 160+/−5); or B6CBA-Tg(HDexon1)62Gpb/1J) include the 1 kb 5′-UTR, exon I and intron 260 bp of human HTT, which are inserted into one other gene (e.g., Gm12695, chromosome 4, chr4:96,409,585-96,414,930).

Accordingly, in order to test a specific miR, the selected mouse model should comprise the target region of HTT (see e.g., FIG. 5-6, Tables 1 and 3). As a non-limiting example, the transgene mouse model Hu128, having a knock in of the full-size human HTT gene, can be used to test a miR targeting any one of regions I-V (e.g., SEQ ID NOs: 1-24). As another non-limiting example, other transgene mouse models (e.g., B6CBA-R6/2 or B6CBA-Tg(HDexon1) strains), having the 1 kb 5′-UTR, exon I and intron 260 bp of human HTT, can be used to test a miR targeting any one of regions I-III (e.g., SEQ ID NOs: 1-10).

1. (canceled)

2. An isolated nucleic acid comprising:
(a) a first region comprising a first adeno-associated virus (AAV) inverted terminal repeat (ITR), or a variant thereof; and
(b) a second region comprising a transgene encoding one or more miRNAs, wherein each miRNA is encoded by a sequence comprising the sequence set forth in any one of SEQ ID NOs: 1-22 flanked by a miRNA backbone sequence.

3. The isolated nucleic acid of claim 2, wherein the transgene comprises two miRNA or two precursor miRNAs in tandem that are flanked by introns.

4. The isolated nucleic acid of claim 3, wherein the flanking introns are identical or are from the same species.

5. (canceled)

6. The isolated nucleic acid of claim 3, wherein the flanking introns are hCG introns.

7. The isolated nucleic acid of claim 2, wherein the transgene comprises a promoter.

8. The isolated nucleic acid of claim 7, wherein the promoter is a synapsin (Syn1) promoter.

9. The isolated nucleic acid of claim 2, wherein the transgene further encodes a protein.

10. The isolated nucleic acid of claim 9, wherein the protein is CYP46A1.

11. The isolated nucleic acid of claim 2, wherein the one or more miRNAs are located in an untranslated portion of the transgene.

12. The isolated nucleic acid of claim 11, wherein the untranslated portion is an intron.

13. The isolated nucleic acid of claim 11, wherein the untranslated portion is between the last codon of the nucleic acid sequence encoding a protein and a poly-A tail sequence, or between the last nucleotide base of a promoter sequence and a poly-A tail sequence.

14. The isolated nucleic acid of claim 2, further comprising a third region comprising a second adeno-associated virus (AAV) inverted terminal repeat (ITR), or a variant thereof.

15. The isolated nucleic acid of claim 2, wherein the ITR lacks a functional terminal resolution site (TRS).

16. The isolated nucleic acid of claim 2, wherein at least one of the miRNAs hybridizes with and inhibits expression of human huntingtin (SEQ ID NO: 25).

17. A vector comprising the isolated nucleic acid of claim 2.

18. (canceled)

19. A host cell comprising the isolated nucleic acid of claim 2.

20. A recombinant AAV (rAAV) comprising:
(a) a capsid protein; and
(b) the isolated nucleic acid of claim 2.

21.-25. (canceled)

26. A composition comprising an isolated nucleic acid of claim 2.

27.-28. (canceled)

29. A method for treating Huntington's disease in a subject in need thereof, the method comprising administering to a subject having or at risk of developing Huntington's disease a therapeutically effective amount of the isolated nucleic acid of claim 2.

30.-36. (canceled)

37. The isolated nucleic acid of claim 2, wherein each miRNA comprises a seed sequence complementary to SEQ ID NO: 25.


SEQUENCE LISTING (36 sequences)

SEQ ID NO: 1 - DNA, 20 bp, Artificial Sequence
  synthetic polynucleotide
gctgctgctgctgctgctgc

SEQ ID NO: 2 - DNA, 20 bp, Artificial Sequence
  synthetic polynucleotide
tgctgctgctgctgctgctg

SEQ ID NO: 3 - DNA, 20 bp, Artificial Sequence
  synthetic polynucleotide
ggcggcggcggcggcggcgg

SEQ ID NO: 4 - DNA, 20 bp, Artificial Sequence
  synthetic polynucleotide
tgctggaaggacttgaggga

SEQ ID NO: 5 - DNA, 20 bp, Artificial Sequence
  synthetic polynucleotide
tgttgctgctgctgctgctg

SEQ ID NO: 6 - DNA, 20 bp, Artificial Sequence
  synthetic polynucleotide
cgaggccggggcggggcaca

SEQ ID NO: 7 - DNA, 20 bp, Artificial Sequence
  synthetic polynucleotide
cggggcggggccgtggaggg

SEQ ID NO: 8 - DNA, 20 bp, Artificial Sequence
  synthetic polynucleotide
actgtgccactatgttttca

SEQ ID NO: 9 - DNA, 20 bp, Artificial Sequence
  synthetic polynucleotide
gccttcatcagcttttccag

SEQ ID NO: 10 - DNA, 20 bp, Artificial Sequence
  synthetic polynucleotide
gaggggtggggaggctgggg

SEQ ID NO: 11 - DNA, 20 bp, Artificial Sequence
  synthetic polynucleotide
tccttgacctgctgctgcag

SEQ ID NO: 12 - DNA, 20 bp, Artificial Sequence
  synthetic polynucleotide
ccttccactggccatgatgc

SEQ ID NO: 13 - DNA, 20 bp, Artificial Sequence
  synthetic polynucleotide
actgtgccactatgttttca

SEQ ID NO: 14 - DNA, 20 bp, Artificial Sequence
  synthetic polynucleotide
tgaggtatcagattgtctag

SEQ ID NO: 15 - DNA, 20 bp, Artificial Sequence
  synthetic polynucleotide
aaattaatctcttacctgat

SEQ ID NO: 16 - DNA, 20 bp, Artificial Sequence
  synthetic polynucleotide
cccagggctagcaaggaaca

SEQ ID NO: 17 - DNA, 20 bp, Artificial Sequence
  synthetic polynucleotide
aattcagtagcttcccttaa

SEQ ID NO: 18 - DNA, 20 bp, Artificial Sequence
  synthetic polynucleotide
ctgggcccgcagcggaaggg

SEQ ID NO: 19 - DNA, 20 bp, Artificial Sequence
  synthetic polynucleotide
ttattgctgtctactatccg

SEQ ID NO: 20 - DNA, 20 bp, Artificial Sequence
  synthetic polynucleotide
tcagtccttcccaaagctct

SEQ ID NO: 21 - DNA, 20 bp, Artificial Sequence
  synthetic polynucleotide
taatctctttactgatataa

SEQ ID NO: 22 - DNA, 20 bp, Artificial Sequence
  synthetic polynucleotide
tcagcagtgttatttcttac

SEQ ID NO: 23 - DNA, 22 bp, Artificial Sequence
  synthetic polynucleotide
aaaccgttaccattactgagtt

SEQ ID NO: 24 - DNA, 21 bp, Artificial Sequence
  synthetic polynucleotide
aaatcgctgatttgtgtagtc

SEQ ID NO: 25 - DNA, 13498 bp, Homo sapiens
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatcaaaaaaaaaaaaaaaaaa

SEQ ID NO: 26 - DNA, 1502 bp, Homo sapiens
tgagccccgggctgctgctgctcggcagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttttggattgggctaagaagtatggacctgttgtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtcatagacctggccttcagccggagctccttggttagcttaatggaaacattcaacgagaaggctgagcagctggtggagattctagaagccaaggcagatgggcagaccccagtgtccatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaggtcattggttctaagaggtacctggatttcgaggacctggggagactgcagtacctgtcccaggtcctcaaagagtcgctgaggctgtacccaccagcatggggcacctttcgcctgctggaagaggagaccttgattgatggggtcagagtccccggcaacaccccgctcttgttcagcacctatgtcatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgctga

SEQ ID NO: 27 - protein, 500 aa, Homo sapiens
MSPGLLLLGSAVLLAFGLCCTFVHRARSRYEHIPGPPRPSFLLGHLPCFWKKDEVGGRVLQDVFLDWAKKYGPVVRVNVFHKTSVIVTSPESVKKFLMSTKYNKDSKMYRALQTVFGERLFGQGLVSECNYERWHKQRRVIDLAFSRSSLVSLMETFNEKAEQLVEILEAKADGQTPVSMQDMLTYTAMDILAKAAFGMETSMLLGAQKPLSQAVKLMLEGITASRNTLAKFLPGKRKQLREVRESIRFLRQVGRDWVQRRREALKRGEEVPADILTQILKAEEGAQDDEGLLDNFVTFFIAGHETSANHLAFTVMELSRQPEIVARLQAEVDEVIGSKRYLDFEDLGRLQYLSQVLKESLRLYPPAWGTFRLLEEETLIDGVRVPGNTPLLFSTYVMGRMDTYFEDPLTFNPDRFGPGAPKPRFTYFPFSLGHRSCIGQQFAQMEVKVVMAKLLQRLEFRLVPGQRFGLQEQATLKPLDPVLCTLRPRGWQPAPPPPPC

SEQ ID NO: 28 - DNA, 1653 bp, Artificial Sequence
  synthetic polynucleotide
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 29 - protein, 550 aa, Artificial Sequence
  synthetic polypeptide
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 30 - DNA, 417 bp, Artificial Sequence
  synthetic polynucleotide
acgcagcagcagcagcagcagcacagcagcagcagcagcagcagaccagcagcagcagcagcagacccgccgccgccgccgccgccactccctcaagtccttccagcaaccagcagcagcagcagcaacaactgtgccccgccccggcctcgacccctccacggccccgccccgactgaaaacatagtggcacagtacctggaaaagctgatgaaggcacccccagcctccccacccctcacattttaatgaaaccagggtaacttatatcagtaaagagattaaccctaggacagatctactccggaacgagctcacgactctagatcataatcagccataccacatttgtagaggttttacttgctttaaaaaacctcccacacctccccctgaacctgaaacataaaatgaatg

SEQ ID NO: 31 - DNA, 477 bp, Homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaggcgcgccgagctcggatcctgacg

SEQ ID NO: 32 - DNA, 448 bp, Homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 33 - DNA, 204 bp, Artificial Sequence
  synthetic polynucleotide
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagct

SEQ ID NO: 34 - DNA, 635 bp, Homo sapiens
ctagcacatcgatacggtacccaccgatattatttgcccgatggtatccccgtttacaggtaagaagatctggcgcgcctcactagtacctcgagattacgaagatatcttacctgagtcgacaccctaggacagatcttccggactgggcaccttccacctccttccaggcaatcactggcatgagaaggggcagaccagtgtgagctgtggaaggacgcctctttctggaggagtgtgacccccagtaagcttcacgtggggcagttcctgagggtggggatctgaaatgttggggtatctcaggtccctcgggctgtggggtgggctctgaaaggcaggtgtccgggtggtgggtcctgaataggagatgccgggaagggtctctgggtctttgtgggtggtgtaccctgggggatgggaaggccggggctcagggctgtggtctcaggcccgggtgaagcagtgtccttgtccggttaccctgcagggcggcttcgtctgggttccgtttatccgggcaaaccgggcccgcgactctagatcataatcagccataccacatttgtagaggttttacttgctttaaaaaacctcccacacctccccctgaacctgaaacataaaatgaatgc

SEQ ID NO: 35 - DNA, 386 bp, Artificial Sequence
  synthetic polynucleotide
agattacttcttcaggttaacccaacagaaggctaaagaaggtatattgctgttgacagtgagcgacgcagcagcagcagcagcagctagtgaagccacagatgtagctgctgctgctgctgctgcgctgcctactgcctcggacttcaaggggctactttaggagcaattatcttgtttactaaaactgaagatatcttacttcttcaggttaacccaacagaaggctaaagaaggtatattgctgttgacagtgagcgacgcagcagcagcagcagcagctagtgaagccacagatgtagctgctgctgctgctgctgcgctgcctactgcctcggacttcaaggggctactttaggagcaattatcttgtttactaaaactga

SEQ ID NO: 36 - DNA, 54 bp, Artificial Sequence
  synthetic polynucleotide
aggcctaataaagagctcagatgcatcgatcagagtgtgttggttttttgtgtg